(19) United States
(12) Patent Application Publication
(10) Pub. No.: US 2022/0267737 A1
(43) Pub. Date: Aug. 25, 2022

(54) CELL-TYPE SELECTIVE IMMUNOPROTECTION OF CELLS

(71) Applicant: UNIVERSITY OF ROCHESTER, Rochester, NY (US)

(72) Inventors: Steven A. GOLDMAN, Webster, NY (US); Abdellatif BENRAISS, Astoria, NY (US); Christina TROJEL-HANSEN, Rochester, NY (US)

(21) Appl. No.: 17/627,894

(22) PCT Filed: Jul. 20, 2020
(86) PCT No.: PCT/US2020/042768

§ 371 Date: Jan. 18, 2022

Related U.S. Application Data
(60) Provisional application No. 62/875,883, filed on Jul. 18, 2019.

(51) Int. Cl. C12N 5/074; C07K 14/705; C12N 15/113

(52) U.S. Cl. CPC C12N 5/0696; C07K 14/70503; C12N 2310/20; C07K 14/70532; C12N 2510/00; C12N 15/1138

(57) ABSTRACT

The present disclosure is directed to preparation of one or more cells, wherein cells of the preparation are modified to conditionally express (i) increased levels of one or more immune checkpoint proteins as compared to corresponding wild-type cells, (ii) reduced levels of one or more HLA-I proteins as compared to corresponding wild-type cells, or a combination of (i) and (ii). The present disclosure is further directed to methods and constructs for producing the cell preparations as well as methods of administering the cell preparation to a subject in need thereof.

This application claims priority benefit of U.S. Provisional Patent Application No. 62/875,883, filed Jul. 18, 2019, which is hereby incorporated by reference in its entirety.

### FIELD OF THE DISCLOSURE

This present disclosure relates to methods of selectively inducing immunoprotection of terminally differentiated cells, and cell preparations that can be selectively immunoprotected.

### BACKGROUND

The acute phase of transplant rejection can occur within about 1-3 weeks and usually involves the action of host T cells on donor tissues due to sensitization of the host system to the donor human leukocyte antigen class I (HLA-I) and human leukocyte antigen class II (HLA-II) molecules. In most cases, the triggering antigens are the HLA-I proteins. For best success, non-autologous donor cells are typed for HLA and matched to the transplant recipient as completely as possible. However, even between family members, which can share a high percentage of HLA identity, allogenic donations are often unsuccessful. To prevent rejection, allogenic transplant recipients are often subjected to profound immunosuppressive therapy, which can lead to complications and significant morbidities due to opportunistic infections. Thus, the recognition of non-self HLA-I and non-self HLA-II proteins is a major hurdle in allogenic cell transplantation and cell replacement therapies.

The surface expression of the HLA-I or HLA-II genes can be modulated by tumor cells and viral pathogens. For example, the downregulation of β₂-microglobulin (B2M), which forms a heterodimer with the HLA-Iα chain, is a widespread mechanism used by tumor cells to escape the antitumor-mediated immune response (Nomura et al., “β2-Microglobulin-mediated Signaling as a Target for Cancer Therapy,” Anticancer Agents Med Chem. 14(3):343-352 (2014), which is hereby incorporated by reference in its entirety). In another example, infection of certain cell types with alpha- or beta-herpesviruses, such as HSV and HCMV, results in reduced surface expression of HLA-I and HLA-II complexes through proteosomal degradation of HLA-I heavy chains and HLA-IIα chains (HLA-DRα and HLA-DMα) (Wiertz et al., “Herpesvirus Interference with Major Histocompatibility Complex Class II-Restricted T-Cell Activation,” J. Virology 81(9):4389-4386 (2007)).

Importantly, in the context of non-autologous cell transplantation, the down regulation or absence of HLA-I and HLA-II molecules on the surface of donor cells may leave such cells susceptible to clearance by the innate immune system. For example, natural killer (NK) cells monitor infections in a host by recognizing and inducing apoptosis in cells that do not express HLA-I molecules. Likewise, macrophages resident in the spleen and liver target autologous cells which fail to present ‘self’ proteins for clearance by programmed cell phagocytosis (Krysoko et al., “Macrophages Regulate the Clearance of Living Cells by Calreticulin,” Nature Comm. 9, Article Number: 4644 (2018)).

Another consideration for cell transplantation and cell replacement therapies, is the use of non-terminally differentiated cells, such as pluripotent (e.g., embryonic stem cells and induced pluripotent stem cells) or multipotent stem cells. Such cells may be transplanted as allogenic (donor-derived) stem cells or autologous (self-derived) stem cells. Since undifferentiated stem cells are characterized by the capacity for rapid growth with low rates of spontaneous differentiation, a concern exists regarding the risk of tumorigenesis, both immediately and long-term after stem cell transplantation (Mousavinejad et al., “Current Biosafety Considerations in Stem Cell Therapy,” Cell J. 18(2):281-287 (2016)).

The present disclosure is directed to overcoming deficiencies in the art.

### SUMMARY

One aspect of the disclosure relates to a recombinant genetic construct comprising a first gene sequence expressed in a cell-type specific manner, one or more immune checkpoint protein encoding nucleotide sequences positioned 3′ to the first gene sequence, and a second gene sequence expressed in a cell-type specific manner, where the second gene sequence is located 3′ to the one or more immune checkpoint protein encoding nucleotide sequences.

Another aspect of the disclosure relates to a recombinant genetic construct comprising a first gene sequence expressed in a cell-type specific manner, a nucleotide sequence encoding one or more agents that reduce expression of one or more HLA-I molecules, said nucleotide sequence positioned 3′ to the first gene sequence, and a second gene sequence expressed in a cell-type specific manner, wherein the second gene sequence is located 3′ to the nucleotide sequence encoding one or more agents that reduce expression of one or more HLA-I molecules.

Another aspect of the disclosure relates to a recombinant genetic construct comprising a first gene sequence expressed in a cell-type specific manner; one or more immune checkpoint protein encoding nucleotide sequences; a nucleotide sequence encoding one or more agents that reduce expression of one or more HLA-I molecules, wherein said immune checkpoint protein encoding nucleotide sequences and said nucleotide sequence encoding one or more agents that reduce expression of one or more HLA-I molecules are positioned 3′ to the first gene sequence. The recombinant genetic construct further comprises a second gene sequence expressed in a cell-type specific manner, wherein the second gene sequence is located 3′ to the one or more immune checkpoint protein encoding nucleotide sequences and the nucleotide sequence encoding one or more agents that reduce expression of one or more HLA-I molecules.

Another aspect of the disclosure relates to a preparation of one or more cells comprising a recombinant genetic construct of the present disclosure.

A further aspect relates to a method that involves administering the preparation of one or more cells comprising the recombinant genetic construct of the present disclosure to a subject in need thereof.

Yet another aspect of the disclosure relates to a method of treating a subject having a condition mediated by a loss of myelin or dysfunction or loss of oligodendrocytes. This method involves administering, to the subject, the preparation of one or more cells comprising the recombinant genetic construct as described herein under conditions effective to treat the condition.

Another aspect relates to a method of treating a subject having a condition mediated by dysfunction or loss of astrocytes. This method involves administering, to the subject, the preparation of one or more cells comprising the recombinant genetic construct as described herein under conditions effective to treat the condition.

Another aspect relates to a method of treating a subject having a condition mediated by dysfunction or loss of neurons. This method involves administering, to the subject, a preparation of one or more cells comprising the recombinant genetic construct as described herein under conditions effective to treat the condition.

An additional aspect relates to a preparation of one or more cells, where cells of the preparation are modified to conditionally express increased levels of one or more immune checkpoint proteins as compared to corresponding wild-type cells, conditionally express reduced levels of one or more endogenous HLA-I proteins as compared to corresponding wild-type cells, or to conditionally express increased levels of one or more immune checkpoint proteins and express reduced levels of one or more endogenous HLA-I proteins as compared to corresponding wild-type cells.

Yet another embodiment relates to a method of generating a conditionally immunoprotected cell. This method involves modifying a cell to conditionally express increased levels of one or more immune checkpoint proteins, modifying the cell to conditionally express one or more agents that reduce expression of one or more endogenous HLA-proteins, or modifying a cell to conditionally express increased levels of one or more immune checkpoint proteins and to conditionally express one or more agents that reduce expression of one or more endogenous HLA-proteins.

### BRIEF DESCRIPTION OF THE DRAWINGS

FIG. 1 is a schematic illustration of a recombinant genetic construct of the present disclosure comprising (i) first and second gene sequences that are expressed in a cell-type specific manner, (ii) one or more immune checkpoint proteins encoding nucleotide sequences, and (iii) a nucleotide sequence encoding one or more agents that reduce expression of one or more HLA-I molecules. As shown in this schematic, an exemplary recombinant genetic construct may comprise, 5′→3′, a first gene sequence expressed in a cell-type specific manner (i.e., a 5′ homology arm), a self-cleaving peptide encoding nucleotide sequence (e.g., P2a), an immune checkpoint protein encoding nucleotide sequence, a stop codon, a nucleotide sequence encoding an agent that reduces expression of one or more HLA-I molecules (i.e., an shRNA), a selection marker, and a second gene sequence expressed in the same cell-type specific manner as the first gene sequence (i.e., a 3′ homology arm).

FIG. 2 is a schematic illustration of a recombinant genetic construct expressed in a cell-type specific matter where the construct comprises a HLA-E/syB2M knock-in vector and shRNAs targeting B2M and CIITA. This exemplary recombinant genetic construct comprises, 5′→3′, a first gene sequence expressed in a cell-type specific manner (i.e., a 5′ homology arm), a self-cleaving peptide encoding nucleotide sequence (e.g., P2a), an immune checkpoint protein encoding nucleotide sequence (e.g., HLA-E/syB2M), a stop codon, a nucleotide sequence encoding an agent that reduces expression of one or more HLA-I molecules (i.e., anti-B2M shRNA), a nucleotide sequence encoding one or more agents that reduce expression of one or more HLA-II molecules (i.e., anti-CIITA shRNA), a selection marker (Puromycin), and a second gene sequence expressed in a the same cell-type specific manner as the first gene sequence (i.e., a 3′ homology arm). The selection marker shown in this example comprises an EF1a promoter and a polyadenylation signal (PA).

FIG. 3 is a schematic illustration of a recombinant genetic construct expressed in a cell-type specific manner that comprises a CD47 knock-in vector and shRNAs targeting B2M and CIITA. The recombinant genetic construct comprises, 5′→3′, a first gene sequence expressed in a cell-type specific manner (i.e., a 5′ homology arm), a self-cleaving peptide encoding nucleotide sequence (e.g., P2a), an immune checkpoint protein encoding nucleotide sequence (i.e., CD47), a stop codon, a nucleotide sequence encoding one or more agents that reduce expression of one or more HLA-I molecules (i.e., anti-B2M shRNA), a nucleotide sequence encoding one or more agents that reduce expression of one or more HLA-II molecules (i.e., anti-CIITA shRNA), a selection marker (Puromycin), and a second gene sequence expressed in the same cell-type specific manner as the first gene sequence (i.e., a 3′ homology arm). The selection marker shown in this example comprises an EF1a promoter and a polyadenylation signal (PA).

FIG. 4 is a schematic illustration of a recombinant genetic construct expressed in a cell-type specific manner that comprises a PD-L1 knock-in vector and shRNAs targeting B2M and CIITA. The recombinant genetic construct comprises, 5′→3′, a first gene sequence expressed in a cell-type specific manner (i.e., a 5′ homology arm), a self-cleaving peptide encoding nucleotide sequence (e.g., P2a), an immune checkpoint protein encoding nucleotide sequence (i.e., PD-L1), a stop codon, a nucleotide sequence encoding one or more agents that reduce expression of one or more endogenous HLA-I molecules (i.e., anti-B2M shRNA), a nucleotide sequence encoding one or more agents that reduce expression of one or more endogenous HLA-II molecules (i.e., anti-CIITA shRNA), a selection marker (Puromycin), and a second gene sequence expressed in the same cell-type specific manner as the first gene sequence (i.e., a 3′ homology arm). The selection marker shown in this example comprises an EF1a promoter and a polyadenylation signal (PA).

FIG. 5 is a matrix showing combinations of targeted cells and protective signals (i.e., immune checkpoint proteins). Suitable cell targets include oligodendrocyte progenitor cells (MYRF locus), neurons (SYN1 locus), and astrocytes (GFAP locus). Immune checkpoint proteins, also referred to herein as “Protective signals” or “don't eat me signals”, include HLA-E/syB2M single chain trimer, PD-L1, and CD47. In each permutation shown in the matrix, the knock-in cassettes further comprise a nucleotide sequence encoding an anti-B2M shRNA (to deplete expression of endogenous HLA-I/B2M complexes) and/or an anti-CIITA shRNA (to deplete expression of HLA-II complexes).

FIG. 6 is a schematic illustration of an exemplary recombinant genetic construct comprising a HLA-E/syB2M knock-in vector targeting the synapsin (SYN1) gene locus, which is restrictively expressed in neurons. The recombinant genetic construct comprises, 5′→3′, a 5′ homology arm (a first nucleotide sequence of the synapsin 1 gene), a self-cleaving peptide encoding nucleotide sequence (e.g., P2a), a nucleotide sequence encoding HLA-E/syB2M, a stop codon, a polyadenylation signal (PA), a nucleotide sequence encoding anti-B2M shRNA, a nucleotide sequence encoding anti-CIITA shRNA, a puromycin selection marker, and a 3′ homology arm (a second nucleotide sequence of the synapsin 1 gene). The selection marker in this exemplary construct comprises an EF1a promoter and a polyadenylation signal (PA).

FIG. 7 is a schematic illustration of an exemplary recombinant genetic construct comprising a CD47 knock-in vector targeting the synapsin (SYN1) gene locus, which is restrictively expressed in neurons. The recombinant genetic construct comprises, 5′→3′, a 5′ homology arm (a first nucleotide sequence of the synapsin 1 gene), a self-cleaving peptide encoding nucleotide sequence (e.g., P2a), a nucleotide sequence encoding CD47, a stop codon, a polyadenylation signal (PA), a nucleotide sequence encoding anti-B2M shRNA, a nucleotide sequence encoding anti-CIITA shRNA, a puromycin selection marker, and a 3′ homology arm (a second nucleotide sequence of the synapsin 1 gene). The selection marker in this exemplary construct comprises an EF1a promoter and a polyadenylation signal (PA).

FIG. 8 is a schematic illustration of an exemplary recombinant genetic construct comprising a PD-L1 knock-in vector targeting the synapsin (SYN1) gene locus, which is expressed in neurons. The recombinant genetic construct comprises, 5′→3′, a 5′ homology arm (a first nucleotide sequence of the synapsin 1 gene), a self-cleaving peptide encoding nucleotide sequence (e.g., P2a), a nucleotide sequence encoding PD-L1, a stop codon, a polyadenylation signal (PA), a nucleotide sequence encoding anti-B2M shRNA, a nucleotide sequence encoding anti-CIITA shRNA, a puromycin selection marker, and a 3′ homology arm (a second nucleotide sequence of the synapsin 1 gene). The selection marker in this exemplary construct comprises an EF1a promoter and a polyadenylation signal (PA).

FIG. 9 is a schematic illustration of a recombinant genetic construct comprising a HLA-E/syB2M knock-in vector targeting the myelin regulatory factor (MYRF) gene locus, which is expressed in oligodendrocyte progenitor cells and oligodendrocytes. The recombinant genetic construct comprises a 5′ homology arm (a first nucleotide sequence of the myelin regulatory factor gene), a self-cleaving peptide encoding nucleotide sequence (e.g., P2a), a nucleotide sequence encoding HLA-E/syB2M, a stop codon, a polyadenylation signal (PA), a nucleotide sequence encoding anti-B2M shRNA, a nucleotide sequence encoding anti-CIITA shRNA, a puromycin selection marker, and a 3′ homology arm (a second nucleotide sequence of the myelin regulatory factor gene). The selection marker in this exemplary construct comprises an EF1a promoter and a polyadenylation signal (PA).

FIG. 10 is a schematic illustration of an exemplary recombinant genetic construct comprising a CD47 knock-in vector targeting the myelin regulatory factor (MYRF) gene locus, which is restrictively expressed in oligodendrocyte progenitor cells and oligodendrocytes. The recombinant genetic construct comprises, 5′→3′, a 5′ homology arm (a first nucleotide sequence of the myelin regulatory factor gene), a self-cleaving peptide encoding nucleotide sequence (e.g., P2a), a nucleotide sequence encoding CD47, a stop codon, a polyadenylation signal (PA), a nucleotide sequence encoding anti-B2M shRNA, a nucleotide sequence encoding anti-CIITA shRNA, a puromycin selection marker, and a 3′ homology arm (a second nucleotide sequence of the myelin regulatory factor gene). The selection marker in this exemplary construct comprises an EF1a promoter and a polyadenylation signal (PA) for constitutive expression in mammalian cells.

FIG. 11 is a schematic illustration of an exemplary recombinant genetic construct comprising a PD-L1 knock-in vector targeting the myelin regulatory factor (MYRF) gene locus, which is restrictively expressed in oligodendrocyte progenitor cells and oligodendrocytes. The recombinant genetic construct comprises, 5′→3′, a 5′ homology arm (a first nucleotide sequence of the myelin regulatory factor gene), a self-cleaving peptide encoding nucleotide sequence (e.g., P2a), a nucleotide sequence encoding PD-L1, a stop codon, a polyadenylation signal (PA), a nucleotide sequence encoding anti-B2M shRNA, a nucleotide sequence encoding anti-CIITA shRNA, a puromycin selection marker, and a 3′ homology arm (a second nucleotide sequence of the myelin regulatory factor gene). The selection marker in this exemplary construct comprises an EF1a promoter and a polyadenylation signal (PA) for constitutive expression in mammalian cells.

FIG. 12 is a schematic illustration of an exemplary recombinant genetic construct comprising a HLA-E/syB2M knock-in vector targeting the glial fibrillary acidic protein (GFAP) gene locus, which is restrictively expressed in astrocytes. The recombinant genetic construct comprises, 5′→3′, a 5′ homology arm (a first nucleotide sequence of the glial fibrillary acidic protein gene), a self-cleaving peptide encoding nucleotide sequence (e.g., P2a), a nucleotide sequence encoding HLA-E/syB2M, a stop codon, a polyadenylation signal (PA), a nucleotide sequence encoding anti-B2M shRNA, a nucleotide sequence encoding anti-CIITA shRNA, a puromycin selection marker, and a 3′ homology arm (a second nucleotide sequence of the glial fibrillary acidic protein gene). The selection marker in this exemplary construct comprises an EF1a promoter and a polyadenylation signal (PA) for constitutive expression in mammalian cells.

FIG. 13 is a schematic illustration of an exemplary recombinant genetic construct comprising a CD47 knock-in vector targeting the glial fibrillary acidic protein (GFAP) gene locus, which is restrictively expressed in astrocytes. The recombinant genetic construct comprises, 5′→3′, a 5′ homology arm (a first nucleotide sequence of the glial fibrillary acidic protein gene), a self-cleaving peptide encoding nucleotide sequence (e.g., P2a), a nucleotide sequence encoding CD47, a stop codon, a polyadenylation signal (PA), a nucleotide sequence encoding anti-B2M shRNA, a nucleotide sequence encoding anti-CIITA shRNA, a puromycin selection marker, and a 3′ homology arm (a second nucleotide sequence of the glial fibrillary acidic protein gene). The selection marker in this exemplary construct comprises an EF1a promoter and a polyadenylation signal (PA) for constitutive expression in mammalian cells.

FIG. 14 is a schematic illustration of an exemplary recombinant genetic construct comprising a PD-L1 knock-in vector targeting the glial fibrillary acidic protein (GFAP) gene locus, which is restrictively expressed in astrocytes. The recombinant genetic construct comprises, 5′→3′, a 5′ homology arm (a first nucleotide sequence of the glial fibrillary acidic protein gene), a self-cleaving peptide encoding nucleotide sequence (e.g., P2a), a nucleotide sequence encoding PD-L1, a stop codon, a polyadenylation signal (PA), a nucleotide sequence encoding anti-B2M shRNA, a nucleotide sequence encoding anti-CIITA shRNA, a puromycin selection marker, and a 3′ homology arm (a second nucleotide sequence of the glial fibrillary acidic protein gene). The selection marker in this exemplary construct comprises an EF1a promoter and a polyadenylation signal (PA) for constitutive expression in mammalian cells.

FIG. 15 is a schematic illustration of an exemplary recombinant genetic construct comprising a CD47 knock-in vector targeting the myelin regulatory factor (MYRF) gene locus, which is expressed in oligodendrocyte progenitor cells and oligodendrocytes. The recombinant genetic construct comprises a 5′ homology arm (HAL); a self-cleaving peptide encoding nucleotide sequence (P2A); a nucleotide sequence encoding CD47; a nucleotide sequence encoding anti-B2M shRNA; a nucleotide sequence encoding anti-CIITA shRNA; a nucleotide sequence encoding copGFP, a self-cleaving peptide (T2A), and a puromycin resistance gene operatively linked to the EF1a promoter; and a 3′ homology arm (HAR).

FIGS. 16A-16D show the design and validation of a recombinant genetic construct targeting the platelet-derived growth factor receptor alpha (PDGFRA) gene locus. FIG. 16A is a schematic illustration of the strategy and design for a PD-L1 or CD47 knock-in vector (top genetic construct) and a control vector (bottom construct), each targeting the PDGFRA gene locus. The PD-L2 or CD47 knock-in vector comprises, 5′+3′, a 5′ homology arm (a first nucleotide sequence of the platelet-derived growth factor alpha gene), a stop codon, an internal ribosomal entry site (IRES), a nucleotide sequence encoding CD47 or PD-L1, a nucleotide sequence encoding anti-B2M shRNA, a nucleotide sequence encoding anti-CIITA shRNA, a puromycin selection marker, and a 3′ homology arm (a second nucleotide sequence of the platelet-derived growth factor alpha gene). The control vector comprises, 5′→3′, a 5′ homology arm (a first nucleotide sequence of the platelet-derived growth factor alpha gene), a stop codon, an IRES, a nucleotide sequence encoding enhanced Green Fluorescent Protein (EGFP), a stop codon, a puromycin selection marker, and a 3′ homology arm (a second nucleotide sequence of the platelet-derived growth factor alpha gene). The puromycin selection markers in these constructs comprise a phosphoglycerate kinase (PGK) promoter and a polyadenylation signal (PA) for constitutive expression in mammalian cells. FIGS. 16B-16D are fluorescence microscopy images of clones generated using CRISPR-mediated knock-in of PD-L1 (FIG. 16B), CD47 (FIG. 16C), and EGFP (FIG. 16D) using the recombinant genetic constructs targeting the PDGFRA gene locus of FIG. 16A. PD-L1 or CD47, red; DAPI, blue.

FIGS. 17A-17B demonstrate that Human U251 glioma cells expressing CD47 or PD-L1 expand and persist preferentially in immune-humanized hosts. FIG. 17A shows bioluminescent images of human Peripheral Blood Mononuclear Cell-chimerized immunodeficient NOG mice (huPBMC-NOG mice) 1 day, 5 days, and 9 days after subcutaneous injection into the flank of genetically-edited U251 knock-in (KI) cells expressing PD-L1, CD47, or EGFP in the PDGFRA locus (i.e., achieved using the expression vectors of FIG. 16A). FIG. 17B is a graph showing tumor bioluminescence on Day 1, Day 5, and Day 9. FIG. 17B shows that by Day 9, CD47-expressing U251 cells expand and persist to a significantly greater extent than EGFP-expressing control cells, consistent with their avoidance of graft rejection by the humanized host immune system. Treatment effect by 2-way ANOVA (F[2,12)=9.16; p<0.001, n=3 mice/group. Difference between CD47-knock in and EGFP control ** p<0.01 by Sidak post hoc comparison; means±SEM.

### DETAILED DESCRIPTION

The present disclosure relates to a recombinant genetic construct, preparations of one or more cells comprising the recombinant genetic constructs described herein, and methods of treating a subject using the disclosed preparations of cells.

One aspect of the disclosure relates to a recombinant genetic construct that is designed to provide cell-type selective immunoprotection to cells expressing the construct.

In one embodiment, the recombinant genetic construct comprises a first gene sequence expressed in a cell-type specific manner, one or more immune checkpoint protein encoding nucleotide sequences that are positioned 3′ to the first cell specific gene sequence, and a second gene sequence expressed in a cell-type specific manner, where the second gene sequence is located 3′ to the immune checkpoint protein encoding nucleotide sequences.

In another embodiment, the recombinant genetic construct comprises a first gene sequence expressed in a cell-type specific manner, a nucleotide sequence encoding one or more agents that reduce expression of one or more HLA-I molecules, where the nucleotide sequence is positioned 3′ to the first cell specific gene sequence, and a second gene sequence expressed in a cell-type specific manner, where the second gene sequence is positioned 3′ to the nucleotide sequence encoding one or more agents that reduce expression of one or more HLA-I molecules.

In another embodiment, the recombinant genetic construct comprises a first gene sequence expressed in a cell-type specific manner. The recombinant genetic construct further comprises one or more immune checkpoint protein encoding nucleotide sequences coupled to a nucleotide sequence encoding one or more agents that reduce expression of one or more HLA-I molecules, where the immune checkpoint protein encoding nucleotide sequences and the nucleotide sequence encoding one or more agents that reduce expression of one or more HLA-I molecules are positioned 3′ to the first gene sequence. This construct further comprises a second gene sequence expressed in a cell-type specific manner, where the second gene sequence is located 3′ to the immune checkpoint protein encoding nucleotide sequences and the nucleotide sequence encoding one or more agents that reduce expression of one or more HLA-I molecules.

As described in more detail infra, any one of the aforementioned recombinant genetic constructs may also contain a further nucleotide sequence encoding one or more agents that reduce the expression of one or more HLA-II molecules. This further nucleotide sequence may be coupled to the one or more immune checkpoint protein encoding nucleotides sequences, the nucleotide sequence encoding one or more agents that reduce expression of one or more HLA-I molecules, or both.

As used herein, the “recombinant genetic construct” of the disclosure refers to a nucleic acid molecule containing a combination of two or more genetic elements not naturally occurring together. The recombinant genetic construct comprises a non-naturally occurring nucleotide sequence that can be in the form of linear DNA, circular DNA, i.e., placed within a vector (e.g., a bacterial vector, a viral vector), or integrated into a genome.

As described in more detail infra, the recombinant genetic construct is introduced into the genome of cells of interest to effectuate the expression of the one or more immune checkpoint proteins or peptides and/or the one or more agents that reduce expression of one or more HLA-I proteins. In some embodiments, the one or more agents that reduce expression of one or more HLA-I proteins function to reduce surface expression of the one or more HLA-I proteins.

As used herein, the term “nucleotide sequence” and “nucleic acid sequence” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA/RNA hybrids, or a polymer comprising purine and pyrimidine bases or other natural, chemically or biochemically modified, non-natural, or derivatized nucleotide bases. In the context of the recombinant genetic construct of the present disclosure, the nucleotide sequence may be a nucleotide sequence that “encodes” a protein if, in its native state or when manipulated by methods well known to those skilled in the art, the nucleotide sequence can be transcribed and/or translated to produce the mRNA for the protein and/or a fragment thereof. Nucleotide sequences of the recombinant genetic construct may also “encode” an agent that has an effector function if, in it its native state or when manipulated by methods well known in the art, can be transcribed to produce the agent with the desired effector function (e.g., shRNA, siRNA, microRNA, guide RNA, etc.).

The immune checkpoint proteins encoded by the nucleotide sequence of the recombinant genetic construct of the present disclosure can be any protein, or peptide thereof, that is involved in immune system downregulation and/or that promotes immune self-tolerance. In one embodiment, the immune checkpoint protein, or peptide thereof, is one that suppresses the activity of the acquired immune response. In one embodiment, the immune checkpoint protein, or peptide thereof, is one that suppresses the activity of the innate immune response.

In one embodiment, the immune checkpoint protein encoded by the recombinant genetic construct is programmed death ligand 1 (PD-L1), programmed death ligand 2 (PD-L2), or functionally active peptides thereof, that bind to the inhibitory programmed cell death protein 1 (PD-1). PD-1 is primarily expressed on mature T cells in peripheral tissues and the tumor microenvironment. It is also expressed on other non-T cell subsets including B cells, professional APCs, and natural killer (NK) cells. PD-1 signaling is mediated through interaction with its ligands PD-L1 (also known as B7-H1 and CD274) and PD-L2 (also known as B7-DC and CD273). Interaction of PD-1 with any of its ligands, i.e., PD-L1 and PD-L2, transmits an inhibitory signal which reduces the proliferation of CD8⁺ T cells at the lymph nodes, thereby suppressing the immune response.

Suitable nucleotide sequences encoding human PD-L1 and PD-L2 for inclusion in the recombinant genetic construct as described herein are set forth in Table 1 below. Suitable nucleotide sequences also include nucleotide sequences having about 70%, 75%, 80%, 85%, 90%, 95%, 98%, 99%, or 100% sequence identity to the PD-L1 and PD-L2 coding sequences provided in Table 1 below (i.e., SEQ ID NOs. 1-4).

TABLE 1
Suitable PD-L1 and PD-L2 Coding Sequences

| | GenBank | |
|---|---|---|
| | Accession | |
| Name | Number | Sequence |
| Homo | NM_001267706.1 | ATGAGGATATTTGCTGTCTTTATATTCATGACCTACTGGCATTTGCTGA |
| sapiens | | ACGCCCCATACAACAAAATCAACCAAAGAATTTTGGTTGTGGATCCAGT |
| CD274 | | CACCTCTGAACATGAACTGACATGTCAGGCTGAGGGCTACCCCAAGGCC |
| molecule | | GAAGTCATCTGGACAAGCAGTGACCATCAAGTCCTGAGTGGTAAGACCA |
| (CD274), | | CCACCACCAATTCCAAGAGAGAGGAGAAGCTTTTCAATGTGACCAGCAC |
| transcript | | ACTGAGAATCAACACAACAACTAATGAGATTTTCTACTGCACTTTTAGG |
| variant 2, | | AGATTAGATCCTGAGGAAAACCATACAGCTGAATTGGTCATCCCAGAAC |
| mRNA | | TACCTCTGGCACATCCTCCAAATGAAAGGACTCACTTGGTAATTCTGGG |
| | | AGCCATCTTATTATGCCTTGGTGTAGCACTGACATTCATCTTCCGTTTA |
| | | AGAAAAGGGAGAATGATGGATGTGAAAAAATGTGGCATCCAAGATACAA |
| | | ACTCAAAGAAGCAAAGTGATACACATTTGGAGGAGACGTAA (SEQ ID |
| | | NO: 1) |
| Homo | NM_014143.3 | ATGAGGATATTTGCTGTCTTTATATTCATGACCTACTGGCATTTGCTGA |
| sapiens | | ACGCATTTACTGTCACGGTTCCCAAGGACCTATATGTGGTAGAGTATGG |
| CD274 | | TAGCAATATGACAATTGAATGCAAATTCCCAGTAGAAAAACAATTAGAC |
| molecule | | CTGGCTGCACTAATTGTCTATTGGGAAATGGAGGATAAGAACATTATTC |
| (CD274), | | AATTTGTGCATGGAGAGGAAGACCTGAAGGTTCAGCATAGTAGCTACAG |
| transcript | | ACAGAGGGCCCGGCTGTTGAAGGACCAGCTCTCCCTGGGAAATGCTGCA |
| variant 1, | | CTTCAGATCACAGATGTGAAATTGCAGGATGCAGGGGTGTACCGCTGCA |
| mRNA | | TGATCAGCTATGGTGGTGCCGACTACAAGCGAATTACTGTGAAAGTCAA |
| | | TGCCCCATACAACAAAATCAACCAAAGAATTTTGGTTGTGGATCCAGTC |
| | | ACCTCTGAACATGAACTGACATGTCAGGCTGAGGGCTACCCCAAGGCCG |
| | | AAGTCATCTGGACAAGCAGTGACCATCAAGTCCTGAGTGGTAAGACCAC |
| | | CACCACCAATTCCAAGAGAGAGGAGAAGCTTTTCAATGTGACCAGCACA |
| | | CTGAGAATCAACACAACAACTAATGAGATTTTCTACTGCACTTTTAGGA |
| | | GATTAGATCCTGAGGAAAACCATACAGCTGAATTGGTCATCCCAGAACT |
| | | ACCTCTGGCACATCCTCCAAATGAAAGGACTCACTTGGTAATTCTGGGA |
| | | GCCATCTTATTATGCCTTGGTGTAGCACTGACATTCATCTTCCGTTTAA |
| | | GAAAAGGGAGAATGATGGATGTGAAAAAATGTGGCATCCAAGATACAAA |
| | | CTCAAAGAAGCAAAGTGATACACATTTGGAGGAGACGTAA (SEQ ID |
| | | NO: 2) |
| Homo | NM_025239.3 | ATGATCTTCCTCCTGCTAATGTTGAGCCTGGAATTGCAGCTTCACCAGA |
| sapiens | | TAGCAGCTTTATTCACAGTGACAGTCCCTAAGGAACTGTACATAATAGA |
| program | | GCATGGCAGCAATGTGACCCTGGAATGCAACTTTGACACTGGAAGTCAT |
| med cell | | GTGAACCTTGGAGCAATAACAGCCAGTTTGCAAAAGGTGGAAAATGATA |
| death 1 | | CATCCCCACACCGTGAAAGAGCCACTTTGCTGGAGGAGCAGCTGCCCCT |
| ligand 2 | | AGGGAAGGCCTCGTTCCACATACCTCAAGTCCAAGTGAGGGACGAAGGA |
| (PDCD1 | | CAGTACCAATGCATAATCATCTATGGGGTCGCCTGGGACTACAAGTACC |
| LG2), | | TGACTCTGAAAGTCAAAGCTTCCTACAGGAAAATAAACACTCACATCCT |
| mRNA | | AAAGGTTCCAGAAACAGATGAGGTAGAGCTCACCTGCCAGGCTACAGGT |
| | | TATCCTCTGGCAGAAGTATCCTGGCCAAACGTCAGCGTTCCTGCCAACA |
| | | CCAGCCACTCCAGGACCCCTGAAGGCCTCTACCAGGTCACCAGTGTTCT |
| | | GCGCCTAAAGCCACCCCCTGGCAGAAACTTCAGCTGTGTGTTCTGGAAT |
| | | ACTCACGTGAGGGAACTTACTTTGGCCAGCATTGACCTTCAAAGTCAGA |
| | | TGGAACCCAGGACCCATCCAACTTGGCTGCTTCACATTTTCATCCCCTT |
| | | CTGCATCATTGCTTTCATTTTCATAGCCACAGTGATAGCCCTAAGAAAA |
| | | CAACTCTGTCAAAAGCTGTATTCTTCAAAAGACACAACAAAAAGACCTG |
| | | TCACCACAACAAAGAGGGAAGTGAACAGTGCTATCTGA (SEQ ID |
| | | NO: 3) |
| Homo | XM_005251600.3 | ATGATCTTCCTCCTGCTAATGTTGAGCCTGGAATTGCAGCTTCACCAGA |
| sapiens | | TAGCAGCTTTATTCACAGTGACAGTCCCTAAGGAACTGTACATAATAGA |
| program | | GCATGGCAGCAATGTGACCCTGGAATGCAACTTTGACACTGGAAGTCAT |
| med cell | | GTGAACCTTGGAGCAATAACAGCCAGTTTGCAAAAGGTGGAAAATGATA |
| death 1 | | CATCCCCACACCGTGAAAGAGCCACTTTGCTGGAGGAGCAGCTGCCCCT |
| ligand 2 | | AGGGAAGGCCTCGTTCCACATACCTCAAGTCCAAGTGAGGGACGAAGGA |
| (PDCD1 | | CAGTACCAATGCATAATCATCTATGGGGTCGCCTGGGACTACAAGTACC |
| LG2), | | TGACTCTGAAAGTCAAAGCTTCCTACAGGAAAATAAACACTCACATCCT |
| transcript | | AAAGGTTCCAGAAACAGATGAGGTAGAGCTCACCTGCCAGGCTACAGGT |
| variant | | TATCCTCTGGCAGAAGTATCCTGGCCAAACGTCAGCGTTCCTGCCAACA |
| X1 | | CCAGCCACTCCAGGACCCCTGAAGGCCTCTACCAGGTCACCAGTGTTCT |
| mRNA | | GCGCCTAAAGCCACCCCCTGGCAGAAACTTCAGCTGTGTGTTCTGGAAT |
| | | ACTCACGTGAGGGAACTTACTTTGGCCAGCATTGACCTTCAAAGTCAGA |
| | | TGGAACCCAGGACCCATCCAACTTGGCTGCTTCACATTTTCATCCCCTT |
| | | CTGCATCATTGCTTTCATTTTCATAGCCACAGTGATAGCCCTAAGAAAA |
| | | CAACTCTGTCAAAAGCTGTATTCTTCAAAAGACACAACAAAAAGACCTG |
| | | TCACCACAACAAAGAGGGAAGTGAACAGTGCTGTGAATCTGAACCTGTG |
| | | GTCTTGGGAGCCAGGGTGA (SEQ ID NO: 4) |

Additional suitable human PD-L1 encoding nucleotide sequences that can be incorporated in the recombinant genetic construct described herein are known in the art, see e.g., GenBank Accession Nos. BC113734.1, BC113736.1, BC074984.2, and BC069381.1, which are hereby incorporated by reference in their entirety.

Additional suitable human PDL-2 encoding nucleotides sequences that can be incorporated in the recombinant genetic construct described herein are known in the art, see e.g., GenBank Accession Nos. BC113680.1, BC113678.1, and BC074766.2, which are hereby incorporated by reference in their entirety.

In another embodiment, the immune checkpoint protein or peptide encoded by the recombinant genetic construct of the present disclosure is the cell surface antigen, cluster of differentiation 47 (CD47; integrin associated protein (IAP)). The phagocytic activity of macrophages is regulated by activating (“eat”) and inhibitory (“do not eat”) signals. Under normal physiologic conditions, the ubiquitously expressed CD47 suppresses phagocytosis by binding to signal regulatory protein alpha (SIRPα) on macrophages. SIRPα, also known as Src homology 2 domain-containing protein tyrosine phosphatase substrate 1/brain Ig-like molecule with tyrosine-based activation motif/cluster of differentiation antigen-like family member A (SHPS-1/BIT/CD172a), is another membrane protein of the immunoglobulin superfamily that is particularly abundant in the myeloid-lineage hematopoietic cells such as macrophages and dendritic cells. The ligation of SIRPα on phagocytes by CD47 expressed on a neighboring cell results in phosphorylation of SIRPα cytoplasmic immunoreceptor tyrosine-based inhibition (ITIM) motifs, leading to the recruitment of SHP-1 and SHP-2 phosphatases. One resulting downstream effect is the prevention of myosin-IIA accumulation at the phagocytic synapse and consequently inhibition of phagocytosis. Thus, CD47-SIRPα interaction functions as a negative immune checkpoint to send a “don't eat me” signal to ensure that healthy autologous cells are not inappropriately phagocytosed (Lui et al., “Is CD47 an Innate Immune Checkpoint for Tumor Evasion?” J. Hematol. Oncol. 10:12 (2017), which is hereby incorporated by reference in its entirety).

Suitable nucleotide sequences encoding human CD47 for inclusion in the recombinant genetic construct as described herein are set forth in Table 2 below. Suitable nucleotide sequences also include nucleotide sequences having about 70%, 75%, 80%, 85%, 90%, 95%, 98%, 99%, or 100% sequence identity to the CD47 coding sequences provided in Table 2 below (i.e., SEQ ID NOs. 5-8).

TABLE 2
Exemplary CD47 Coding Sequences

| | GenBank | |
|---|---|---|
| | Accession | |
| Name | Number | Sequence |
| Homo | NM_001777.3 | ATGTGGCCCCTGGTAGCGGCGCTGTTGCTGGGCTCGGCGTGCTGCGGA |
| sapiens | | TCAGCTCAGCTACTATTTAATAAAACAAAATCTGTAGAATTCACGTTT |
| CD47 | | TGTAATGACACTGTCGTCATTCCATGCTTTGTTACTAATATGGAGGCA |
| molecule | | CAAAACACTACTGAAGTATACGTAAAGTGGAAATTTAAAGGAAGAGAT |
| (CD47), | | ATTTACACCTTTGATGGAGCTCTAAACAAGTCCACTGTCCCCACTGAC |
| transcript | | TTTAGTAGTGCAAAAATTGAAGTCTCACAATTACTAAAAGGAGATGCC |
| variant 1, | | TCTTTGAAGATGGATAAGAGTGATGCTGTCTCACACACAGGAAACTAC |
| mRNA | | ACTTGTGAAGTAACAGAATTAACCAGAGAAGGTGAAACGATCATCGAG |
| | | CTAAAATATCGTGTTGTTTCATGGTTTTCTCCAAATGAAAATATTCTT |
| | | ATTGTTATTTTCCCAATTTTTGCTATACTCCTGTTCTGGGGACAGTTT |
| | | GGTATTAAAACACTTAAATATAGATCCGGTGGTATGGATGAGAAAACA |
| | | ATTGCTTTACTTGTTGCTGGACTAGTGATCACTGTCATTGTCATTGTT |
| | | GGAGCCATTCTTTTCGTCCCAGGTGAATATTCATTAAAGAATGCTACT |
| | | GGCCTTGGTTTAATTGTGACTTCTACAGGGATATTAATATTACTTCAC |
| | | TACTATGTGTTTAGTACAGCGATTGGATTAACCTCCTTCGTCATTGCC |
| | | ATATTGGTTATTCAGGTGATAGCCTATATCCTCGCTGTGGTTGGACTG |
| | | AGTCTCTGTATTGCGGCGTGTATACCAATGCATGGCCCTCTTCTGATT |
| | | TCAGGTTTGAGTATCTTAGCTCTAGCACAATTACTTGGACTAGTTTAT |
| | | ATGAAATTTGTGGCTTCCAATCAGAAGACTATACAACCTCCTAGGAAA |
| | | GCTGTAGAGGAACCCCTTAATGCATTCAAAGAATCAAAAGGAATGATG |
| | | AATGATGAATAA (SEQ ID NO: 5) |
| Homo | NM_198793.2 | ATGTGGCCCCTGGTAGCGGCGCTGTTGCTGGGCTCGGCGTGCTGCGGA |
| sapiens | | TCAGCTCAGCTACTATTTAATAAAACAAAATCTGTAGAATTCACGTTT |
| CD47 | | TGTAATGACACTGTCGTCATTCCATGCTTTGTTACTAATATGGAGGCA |
| molecule | | CAAAACACTACTGAAGTATACGTAAAGTGGAAATTTAAAGGAAGAGAT |
| (CD47), | | ATTTACACCTTTGATGGAGCTCTAAACAAGTCCACTGTCCCCACTGAC |
| transcript | | TTTAGTAGTGCAAAAATTGAAGTCTCACAATTACTAAAAGGAGATGCC |
| variant 2, | | TCTTTGAAGATGGATAAGAGTGATGCTGTCTCACACACAGGAAACTAC |
| mRNA | | ACTTGTGAAGTAACAGAATTAACCAGAGAAGGTGAAACGATCATCGAG |
| | | CTAAAATATCGTGTTGTTTCATGGTTTTCTCCAAATGAAAATATTCTT |
| | | ATTGTTATTTTCCCAATTTTTGCTATACTCCTGTTCTGGGGACAGTTT |
| | | GGTATTAAAACACTTAAATATAGATCCGGTGGTATGGATGAGAAAACA |
| | | ATTGCTTTACTTGTTGCTGGACTAGTGATCACTGTCATTGTCATTGTT |
| | | GGAGCCATTCTTTTCGTCCCAGGTGAATATTCATTAAAGAATGCTACT |
| | | GGCCTTGGTTTAATTGTGACTTCTACAGGGATATTAATATTACTTCAC |
| | | TACTATGTGTTTAGTACAGCGATTGGATTAACCTCCTTCGTCATTGCC |
| | | ATATTGGTTATTCAGGTGATAGCCTATATCCTCGCTGTGGTTGGACTG |
| | | AGTCTCTGTATTGCGGCGTGTATACCAATGCATGGCCCTCTTCTGATT |
| | | TCAGGTTTGAGTATCTTAGCTCTAGCACAATTACTTGGACTAGTTTAT |
| | | ATGAAATTTGTGGCTTCCAATCAGAAGACTATACAACCTCCTAGGAAT |
| | | AACTGA (SEQ ID NO: 6) |
| Homo | LN680437.1 | ATGTGGCCCCTGGTAGCGGCGCTGTTGCTGGGCTCGGCGTGCTGCGGA |
| sapiens | | TCAGCTCAGCTACTATTTAATAAAACAAAATCTGTAGAATTCACGTTT |
| mRNA | | TGTAATGACACTGTCGTCATTCCATGCTTTGTTACTAATATGGAGGCA |
| for CD47 | | CAAAACACTACTGAAGTATACGTAAAGTGGAAATTTAAAGGAAGAGAT |
| | | ATTTACACCTTTGATGGAGCTCTAAACAAGTCCACTGTCCCCACTGAC |
| | | TTTAGTAGTGCAAAAATTGAAGTCTCACAATTACTAAAAGGAGATGCC |
| | | TCTTTGAAGATGGATAAGAGTGATGCTGTCTCACACACAGGAAACTAC |
| | | ACTTGTGAAGTAACAGAATTAACCAGAGAAGGTGAAACGATCATCGAG |
| | | CTAAAATATCGTGTTGTTTCATGGTTTTCTCCAAATGAAAATATTCTT |
| | | ATTGTTATTTTCCCAATTTTTGCTATACTCCTGTTCTGGGGACAGTTT |
| | | GGTATTAAAACACTTAAATATAGATCCGGTGGTATGGATGAGAAAACA |
| | | ATTGCTTTACTTGTTGCTGGACTAGTGATCACTGTCATTGTCATTGTT |
| | | GGAGCCATTCTTTTCGTCCCAGGTGAATATTCATTAAAGAATGCTACT |
| | | GGCCTTGGTTTAATTGTGACTTCTACAGGGATATTAATATTACTTCAC |
| | | TACTATGTGTTTAGTACAGCGATTGGATTAACCTCCTTCGTCATTGCC |
| | | ATATTGGTTATTCAGGTGATAGCCTATATCCTCGCTGTGGTTGGACTG |
| | | AGTCTCTGTATTGCGGCGTGTATACCAATGCATGGCCCTCTTCTGATT |
| | | TCAGGTTTGAGTATCTTAGCTCTAGCACAATTACTTGGACTAGTTTAT |
| | | ATGAAATTTGTGGAATAA (SEQ ID NO: 7) |
| Synthetic | KJ904432.1 | ATGTGGCCCCTGGTAGCGGCGCTGTTGCTGGGCTCGGCGTGCTGCGGA |
| construct | | TCAGCTCAGCTACTATTTAATAAAACAAAATCTGTAGAATTCACGTTT |
| Homo | | TGTAATGACACTGTCGTCATTCCATGCTTTGTTACTAATATGGAGGCA |
| sapiens | | CAAAACACTACTGAAGTATACGTAAAGTGGAAATTTAAAGGAAGAGAT |
| clone | | ATTTACACCTTTGATGGAGCTCTAAACAAGTCCACTGTCCCCACTGAC |
| ccsbBroadEn_ | | TTTAGTAGTGCAAAAATTGAAGTCTCACAATTACTAAAAGGAGATGCC |
| 13826 | | TCTTTGAAGATGGATAAGAGTGATGCTGTCTCACACACAGGAAACTAC |
| CD47 | | ACTTGTGAAGTAACAGAATTAACCAGAGAAGGTGAAACGATCATCGAG |
| gene, | | CTAAAATATCGTGTTGTTTCATGGTTTTCTCCAAATGAAAATATTCTT |
| encodes | | ATTGTTATTTTCCCAATTTTTGCTATACTCCTGTTCTGGGGACAGTTT |
| complete | | GGTATTAAAACACTTAAATATAGATCCGGTGGTATGGATGAGAAAACA |
| protein | | ATTGCTTTACTTGTTGCTGGACTAGTGATCACTGTCATTGTCATTGTT |
| | | GGAGCCATTCTTTTCGTCCCAGGTGAATATTCATTAAAGAATGCTACT |
| | | GGCCTTGGTTTAATTGTGACTTCTACAGGGATATTAATATTACTTCAC |
| | | TACTATGTGTTTAGTACAGCGATTGGATTAACCTCCTTCGTCATTGCC |
| | | ATATTGGTTATTCAGGTGATAGCCTATATCCTCGCTGTGGTTGGACTG |
| | | AGTCTCTGTATTGCGGCGTGTATACCAATGCATGGCCCTCTTCTGATT |
| | | TCAGGTTTGAGTATCTTAGCTCTAGCACAATTACTTGGACTAGTTTAT |
| | | ATGAAATTTGTGGCTTCCAATCAGAAGACTATACAACCTCCTGGAATA |
| | | ACTG (SEQ ID NO: 8) |

In another embodiment, the immune checkpoint protein encoded by the recombinant genetic construct is CD200. CD200 (also known as OX-2 membrane glycoprotein) is a 45 kDa transmembrane immune checkpoint protein. The CD200 receptor (CD200R) is expressed on cells of the monocyte/macrophage lineage and subsets of B and T cells. Signaling by CD200 prevents normal activation of CD20R bearing myeloid cells, eventuating an immunosuppressive cascade that includes the induction of regulatory T cells (T_{regs}) (Gaiser et al., “Merke Cell Carcinoma Expresses the Immunoregulatory Ligand CD200 and Induces Immunosuppressive Macrophages and Regulatory T Cells,” Oncoimmunology 7(5):e1426517 (2018), which is hereby incorporated by reference in its entirety). For example, CD200 signaling inhibits classic macrophage activation (M1 polarization) and supports an immunosuppressive M2 polarized state that secrets high levels of IL-10, thereby inducing T_{regs}. Thus, cell expression of CD200 via the recombinant genetic construct as described herein, will impart protection to the cell from macrophage and T-cell mediated responses.

Suitable nucleotide sequences encoding human CD200 for inclusion in the recombinant genetic construct as described herein are set forth in Table 3 below. Suitable nucleotide sequences also include nucleotide sequences having about 70%, 75%, 80%, 85%, 90%, 95%, 98%, 99%, or 100% sequence identity to the CD200 coding sequences provided in Table 3 below (i.e., SEQ ID NOs. 9-12).

TABLE 3
Exemplary CD200 Coding Sequences

| | GenBank | |
|---|---|---|
| | Accession | |
| Name | Number | Sequence |
| Homo | NM_005944.7 | ATGGAGAGGCTGGTGATCAGGATGCCCTTCTCTCATCTGTCTACCTACA |
| sapiens | | GCCTGGTTTGGGTCATGGCAGCAGTGGTGCTGTGCACAGCACAAGTGCA |
| CD200 | | AGTGGTGACCCAGGATGAAAGAGAGCAGCTGTACACACCTGCTTCCTTA |
| molecule | | AAATGCTCTCTGCAAAATGCCCAGGAAGCCCTCATTGTGACATGGCAGA |
| (CD200), | | AAAAGAAAGCTGTAAGCCCAGAAAACATGGTCACCTTCAGCGAGAACCA |
| transcript | | TGGGGTGGTGATCCAGCCTGCCTATAAGGACAAGATAAACATTACCCAG |
| variant 1, | | CTGGGACTCCAAAACTCAACCATCACCTTCTGGAATATCACCCTGGAGG |
| mRNA | | ATGAAGGGTGTTACATGTGTCTCTTCAATACCTTTGGTTTTGGGAAGAT |
| | | CTCAGGAACGGCCTGCCTCACCGTCTATGTACAGCCCATAGTATCCCTT |
| | | CACTACAAATTCTCTGAAGACCACCTAAATATCACTTGCTCTGCCACTG |
| | | CCCGCCCAGCCCCCATGGTCTTCTGGAAGGTCCCTCGGTCAGGGATTGA |
| | | AAATAGTACAGTGACTCTGTCTCACCCAAATGGGACCACGTCTGTTACC |
| | | AGCATCCTCCATATCAAAGACCCTAAGAATCAGGTGGGGAAGGAGGTGA |
| | | TCTGCCAGGTGCTGCACCTGGGGACTGTGACCGACTTTAAGCAAACCGT |
| | | CAACAAAGGCTATTGGTTTTCAGTTCCGCTATTGCTAAGCATTGTTTCC |
| | | CTGGTAATTCTTCTCGTCCTAATCTCAATCTTACTGTACTGGAAACGTC |
| | | ACCGGAATCAGGACCGAGAGCCCTAA (SEQ ID NO: 9) |
| Homo | NM_001004196.3 | ATGGAGAGGCTGACTCTGACCAGGACAATTGGGGGCCCTCTCCTTACAG |
| sapiens | | CTACACTCCTAGGAAAGACCACCATCAATGATTACCAGGTGATCAGGAT |
| CD200 | | GCCCTTCTCTCATCTGTCTACCTACAGCCTGGTTTGGGTCATGGCAGCA |
| molecule | | GTGGTGCTGTGCACAGCACAAGTGCAAGTGGTGACCCAGGATGAAAGAG |
| (CD200), | | AGCAGCTGTACACACCTGCTTCCTTAAAATGCTCTCTGCAAAATGCCCA |
| transcript | | GGAAGCCCTCATTGTGACATGGCAGAAAAAGAAAGCTGTAAGCCCAGAA |
| variant 2, | | AACATGGTCACCTTCAGCGAGAACCATGGGGTGGTGATCCAGCCTGCCT |
| mRNA | | ATAAGGACAAGATAAACATTACCCAGCTGGGACTCCAAAACTCAACCAT |
| | | CACCTTCTGGAATATCACCCTGGAGGATGAAGGGTGTTACATGTGTCTC |
| | | TTCAATACCTTTGGTTTTGGGAAGATCTCAGGAACGGCCTGCCTCACCG |
| | | TCTATGTACAGCCCATAGTATCCCTTCACTACAAATTCTCTGAAGACCA |
| | | CCTAAATATCACTTGCTCTGCCACTGCCCGCCCAGCCCCCATGGTCTTC |
| | | TGGAAGGTCCCTCGGTCAGGGATTGAAAATAGTACAGTGACTCTGTCTC |
| | | ACCCAAATGGGACCACGTCTGTTACCAGCATCCTCCATATCAAAGACCC |
| | | TAAGAATCAGGTGGGGAAGGAGGTGATCTGCCAGGTGCTGCACCTGGGG |
| | | ACTGTGACCGACTTTAAGCAAACCGTCAACAAAGGCTATTGGTTTTCAG |
| | | TTCCGCTATTGCTAAGCATTGTTTCCCTGGTAATTCTTCTCGTCCTAAT |
| | | CTCAATCTTACTGTACTGGAAACGTCACCGGAATCAGGACCGAGAGCCC |
| | | TAA (SEQ ID NO: 10) |
| Homo | NM_001318826.1 | ATGAAGGGTGTTACATGTGTCTCTTCAATACCTTTGGTTTTGGGAAGAT |
| sapiens | | CTCAGGAACGGCCTGCCTCACCGTCTATGCCCATAGTATCCCTTCACTA |
| CD200 | | CAAATTCTCTGAAGACCACCTAAATATCACTTGCTCTGCCACTGCCCGC |
| molecule | | CCAGCCCCCATGGTCTTCTGGAAGGTCCCTCGGTCAGGGATTGAAAATA |
| (CD200), | | GTACAGTGACTCTGTCTCACCCAAATGGGACCACGTCTGTTACCAGCAT |
| transcript | | CCTCCATATCAAAGACCCTAAGAATCAGGTGGGGAAGGAGGTGATCTGC |
| variant 3, | | CAGGTGCTGCACCTGGGGACTGTGACCGACTTTAAGCAAACCGTCAACA |
| mRNA | | AAGGCTATTGGTTTTCAGTTCCGCTATTGCTAAGCATTGTTTCCCTGGT |
| | | AATTCTTCTCGTCCTAATCTCAATCTTACTGTACTGGAAACGTCACCGG |
| | | AATCAGGACCGAGAGCCCTAA (SEQ ID NO: 11) |
| Homo | NM_001318828.1 | ATGGTCACCTTCAGCGAGAACCATGGGGTGGTGATCCAGCCTGCCTATA |
| sapiens | | AGGACAAGATAAACATTACCCAGCTGGGACTCCAAAACTCAACCATCAC |
| CD200 | | CTTCTGGAATATCACCCTGGAGGATGAAGGGTGTTACATGTGTCTCTTC |
| molecule | | AATACCTTTGGTTTTGGGAAGATCTCAGGAACGGCCTGCCTCACCGTCT |
| (CD200), | | ATGTACAGCCCATAGTATCCCTTCACTACAAATTCTCTGAAGACCACCT |
| transcript | | AAATATCACTTGCTCTGCCACTGCCCGCCCAGCCCCCATGGTCTTCTGG |
| variant 4, | | AAGGTCCCTCGGTCAGGGATTGAAAATAGTACAGTGACTCTGTCTCACC |
| mRNA | | CAAATGGGACCACGTCTGTTACCAGCATCCTCCATATCAAAGACCCTAA |
| | | GAATCAGGTGGGGAAGGAGGTGATCTGCCAGGTGCTGCACCTGGGGACT |
| | | GTGACCGACTTTAAGCAAACCGTCAACAAAGGCTATTGGTTTTCAGTTC |
| | | CGCTATTGCTAAGCATTGTTTCCCTGGTAATTCTTCTCGTCCTAATCTC |
| | | AATCTTACTGTACTGGAAACGTCACCGGAATCAGGACCGAGAGCCCTAA |
| | | (SEQ ID NO: 12) |

In another embodiment, the immune checkpoint protein encoded by the recombinant genetic construct is CTLA-4. In the immune recognition process, two signals are required for T lymphocyte expansion and differentiation: the T-cell receptor (TCR) binding to the HLA molecule-peptide complex and an antigen-independent costimulatory signal provided by the B7 (CD80 and Cd86)/CD28 interaction. The cytotoxic T-lymphocyte antigen (CTLA-4) is a homologous molecule of CD28 that is a competitive antagonist for B7. CTLA-4 has a greater affinity and avidity for B7 than does CD28, and its translocation to the cell surface after T-cell activation results in B7 sequestration and transduction of a negative signal, responsible for T-cell inactivation (Perez-Garcia et al., “CTLA-4 Polymorphisms and Clinical Outcome after Allogeneic Stem Cell Transplantation from HLA-Identical Sibling Donors,” Blood 110(1):461-7 (2007), which is hereby incorporated by reference in its entirety). Thus, cell expression of CTLA-4 via the recombinant genetic construct as described herein, will impart protection to the cell from cytotoxic T-cell mediated lysis.

The CTLA-4 gene is translated into 2 isoforms: a full-length protein (flCLTA-4) and a soluble counterpart (sCTLA-4), which lacks exon 3 (responsible for coding the transmembrane domain) due to alternative splicing. flCTLA-4 down-regulates T-cell responses by inducing cell-cycle arrest and blocking cytokine production. Thus, in some embodiments, the immune checkpoint protein encoded by the recombinant genetic construct is full length CTLA-4 (flCTLA-4).

Suitable nucleotide sequences encoding human CTLA-4 for inclusion in the recombinant genetic construct as described herein are set forth in Table 4 below. Suitable nucleotide sequences also include nucleotide sequences having about 70%, 75%, 80%, 85%, 90%, 95%, 98%, 99%, or 100% sequence identity to the CTLA-4 coding sequences provided in Table 4 below (i.e., SEQ ID NOs. 13-14 and 44).

TABLE 4
Exemplary CTLA-4 Coding Sequences

| | GenBank | |
|---|---|---|
| | Accession | |
| Name | Number | Sequence |
| Homo | AF414120.1 | ATGGCTTGCCTTGGATTTCAGCGGCACAAGGCTCAGCTGAACCTGG |
| sapiens | | CTACCAGGACCTGGCCCTGCACTCTCCTGTTTTTTCTTCTCTTCAT |
| CTLA4 | | CCCTGTCTTCTGCAAAGCAATGCACGTGGCCCAGCCTGCTGTGGTA |
| (CTLA4) | | CTGGCCAGCAGCCGAGGCATCGCCAGCTTTGTGTGTGAGTATGCAT |
| mRNA, | | CTCCAGGCAAAGCCACTGAGGTCCGGGTGACAGTGCTTCGGCAGGC |
| complete cds | | TGACAGCCAGGTGACTGAAGTCTGTGCGGCAACCTACATGATGGGG |
| | | AATGAGTTGACCTTCCTAGATGATTCCATCTGCACGGGCACCTCCA |
| | | GTGGAAATCAAGTGAACCTCACTATCCAAGGACTGAGGGCCATGGA |
| | | CACGGGACTCTACATCTGCAAGGTGGAGCTCATGTACCCACCGCCA |
| | | TACTACCTGGGCATAGGCAACGGAACCCAGATTTATGTAATTGATC |
| | | CAGAACCGTGCCCAGATTCTGACTTCCTCCTCTGGATCCTTGCAGC |
| | | AGTTAGTTCGGGGTTGTTTTTTTATAGCTTTCTCCTCACAGCTGTT |
| | | TCTTTGAGCAAAATGCTAAAGAAAAGAAGCCCTCTTACAACAGGGG |
| | | TCTATGTGAAAATGCCCCCAACAGAGCCAGAATGTGAAAAGCAATT |
| | | TCAGCCTTATTTTATTCCCATCAATTGA (SEQ ID NO: 13) |
| Homo | NM_005214.5 | ATGGCTTGCCTTGGATTTCAGCGGCACAAGGCTCAGCTGAACCTGG |
| sapiens | | CTACCAGGACCTGGCCCTGCACTCTCCTGTTTTTTCTTCTCTTCAT |
| cytotoxic T- | | CCCTGTCTTCTGCAAAGCAATGCACGTGGCCCAGCCTGCTGTGGTA |
| lymphocyte | | CTGGCCAGCAGCCGAGGCATCGCCAGCTTTGTGTGTGAGTATGCAT |
| associated | | CTCCAGGCAAAGCCACTGAGGTCCGGGTGACAGTGCTTCGGCAGGC |
| protein 4 | | TGACAGCCAGGTGACTGAAGTCTGTGCGGCAACCTACATGATGGGG |
| (CTLA4), | | AATGAGTTGACCTTCCTAGATGATTCCATCTGCACGGGCACCTCCA |
| transcript | | GTGGAAATCAAGTGAACCTCACTATCCAAGGACTGAGGGCCATGGA |
| variant 1, | | CACGGGACTCTACATCTGCAAGGTGGAGCTCATGTACCCACCGCCA |
| mRNA | | TACTACCTGGGCATAGGCAACGGAACCCAGATTTATGTAATTGATC |
| | | CAGAACCGTGCCCAGATTCTGACTTCCTCCTCTGGATCCTTGCAGC |
| | | AGTTAGTTCGGGGTTGTTTTTTTATAGCTTTCTCCTCACAGCTGTT |
| | | TCTTTGAGCAAAATGCTAAAGAAAAGAAGCCCTCTTACAACAGGGG |
| | | TCTATGTGAAAATGCCCCCAACAGAGCCAGAATGTGAAAAGCAATT |
| | | TCAGCCTTATTTTATTCCCATCAATTGA (SEQ ID NO: 14) |
| Homo | NM_001037631.3 | ATGGCTTGCCTTGGATTTCAGCGGCACAAGGCTCAGCTGAACCTGG |
| sapiens | | CTACCAGGACCTGGCCCTGCACTCTCCTGTTTTTTCTTCTCTTCAT |
| cytotoxic T- | | CCCTGTCTTCTGCAAAGCAATGCACGTGGCCCAGCCTGCTGTGGTA |
| lymphocyte | | CTGGCCAGCAGCCGAGGCATCGCCAGCTTTGTGTGTGAGTATGCAT |
| associated | | CTCCAGGCAAAGCCACTGAGGTCCGGGTGACAGTGCTTCGGCAGGC |
| protein 4 | | TGACAGCCAGGTGACTGAAGTCTGTGCGGCAACCTACATGATGGGG |
| (CTLA4), | | AATGAGTTGACCTTCCTAGATGATTCCATCTGCACGGGCACCTCCA |
| transcript | | GTGGAAATCAAGTGAACCTCACTATCCAAGGACTGAGGGCCATGGA |
| variant 2, | | CACGGGACTCTACATCTGCAAGGTGGAGCTCATGTACCCACCGCCA |
| mRNA | | TACTACCTGGGCATAGGCAACGGAACCCAGATTTATGTAATTGCTA |
| | | AAGAAAAGAAGCCCTCTTACAACAGGGGTCTATGTGAAAATGCCCC |
| | | CAACAGAGCCAGAATGTGA (SEQ ID NO: 44) |

In another embodiment, the immune checkpoint protein encoded by the recombinant genetic construct is HLA-E (major histocompatibility complex, class I, E). Natural killer (NK) cells detect infected cells (mainly infected by viruses), foreign cells, or malignant cells in which expression of MHC molecules has decreased, is altered, abolished, or absent. NK cells distinguish normal host cells through the killer cell immunoglobulin-like receptor (KIR) and CD94-NKG2A inhibitory receptors which recognize the MHC class I expressed on the surface of normal host cells. In particular, CD94-NKG2A recognizes HLA-E on the surface of NK cells and CD8⁺ T cells. The binding of these receptors inhibits lysis and cytokine secretion by NK cells. KIRs are also expressed on CD8⁺ T cells and APCs. Thus, cell expression of HLA-E via the recombinant genetic construct as described herein, will impart protection to the cell from NK cell lysis.

Like other HLA class I proteins, HLA-E is a heterodimer consisting of a heavy chain (a chain) and light chain (β₂ microglobulin). In one embodiment, the recombinant genetic construct may comprise a nucleotide sequence encoding the HLA-E (a chain E) and a nucleotide sequence encoding the β₂ microglobulin chain. Alternatively, the recombinant genetic construct may comprises a fusion construct, i.e., a nucleotide sequence encoding a single chain fusion protein that comprises at least a portion of the β₂ microglobulin covalently linked to at least a portion of HLA-E. In other embodiments, the HLA-E/β₂M fusion protein is syβ₂M-HLA-E, where syB2M (synthetic B2M) is expressed as complex with HLA-E. syB2M contains several silent mutations at the target sequence of the shRNA that targets endogenous B2M. As such, syB2M encodes for the exact same protein as wildtype B2M, while being refractory to the shRNA that targets the endogenous B2M only.

Exemplary nucleotide sequences encoding human HLA-E (alpha chain) are provided in Table 5 below. Suitable nucleotide sequences also include nucleotides sequence having about 70%, 75%, 80%, 85%, 90%, 95%, 98%, 99%, or 100% sequence identity to the HLA-E coding sequences provided in Table 5 below (i.e., SEQ ID NOs. 15-17).

TABLE 5
Exemplary HLA-E Coding Sequences

| | GenBank | |
|---|---|---|
| | Accession | |
| Name | Number | Sequence |
| Human | M20022.1 | ATGGTAGATGGAACCCTCCTTTTACTCTCCTCGGAGGCCCTGGCCCTTA |
| HLA-E | | CCCAGACCTGGGCGGGCTCCCACTCCTTGAAGTATTTCCACACTTCCGT |
| Class I | | GTCCCGGCCCGGCCGCGGGGAGCCCCGCTTCATCTCTGTGGGCTACGTG |
| mRNA | | GACGACACCCAGTTCGTGCGCTTCGACAACGACGCCGCGAGTCCGAGGA |
| | | TGGTGCCGCGGGCGCCGTGGATGGAGCAGGAGGGGTCAGAGTATTGGGA |
| | | CCGGGAGACACGGAGCGCCAGGGACACCGCACAGATTTTCCGAGTGAAC |
| | | CTGCGGACGCTGCGCGGCTACTACAATCAGAGCGAGGCCGGGTCTCACA |
| | | CCCTGCAGTGGATGCATGGCTGCGAGCTGGGGCCCGACAGGCGCTTCCT |
| | | CCGCGGGTATGAACAGTTCGCCTACGACGGCAAGGATTATCTCACCCTG |
| | | AATGAGGACCTGCGCTCCTGGACCGCGGTGGACACGGCGGCTCAGATCT |
| | | CCGAGCAAAAGTCAAATGATGCCTCTGAGGCGGAGCACCAGAGAGCCTA |
| | | CCTGGAAGACACATGCGTGGAGTGGCTCCACAAATACCTGGAGAAGGGG |
| | | AAGGAGACGCTGCTTCACCTGGAGCCCCCAAAGACACACGTGACTCACC |
| | | ACCCCATCTCTGACCATGAGGCCACCCTGAGGTGCTGGGCCCTGGGCTT |
| | | CTACCCTGCGGAGATCACACTGACCTGGCAGCAGGATGGGGAGGGCCAT |
| | | ACCCAGGACACGGAGCTCGTGGAGACCAGGCCTGCAGGGGATGGAACCT |
| | | TCCAGAAGTGGGCAGCTGTGGTGGTGCCTTCTGGAGAGGAGCAGAGATA |
| | | CACGTGCCATGTGCAGCATGAGGGGCTACCCGAGCCCGTCACCCTGAGA |
| | | TGGAAGCCGGCTTCCCAGCCCACCATCCCCATCGTGGGCATCATTGCTG |
| | | GCCTGGTTCTCCTTGGATCTGTGGTCTCTGGAGCTGTGGTTGCTGCTGT |
| | | GATATGGAGGAAGAAGAGCTCAGGTGGAAAAGGAGGGAGCTACTCTAAG |
| | | GCTGAGTGGAGCGACAGTGCCCAGGGGTCTGAGTCTCACAGCTTGTAA |
| | | (SEQ ID NO: 15) |
| Human | AJ293263.1 | ATGGTAGATGGAACCCTCCTTTTACTCCTCTCGGAGGCCCTGGCCCTTA |
| MHC | | CCCAGACCTGGGCGGGCTCCCACTCCTTGAAGTATTTCCACACTTCCGT |
| Class I | | GTCCCGGCCCGGCCGCGGGGAGCCCCGCTTCATCTCTGTGGGCTACGTG |
| antigen, | | GACGACACCCAGTTCGTGCGCTTCGACAACGACGCCGCGAGTCCGAGGA |
| HLA- | | TGGTGCCGCGGGCGCCGTGGATGGAGCAGGAGGGGTCAGAGTATTGGGA |
| E*0103 | | CCGGGAGACACGGAGCGCCAGGGACACCGCACAGATTTTCCGAGTGAAT |
| 3 allele | | CTGCGGACGCTGCGCGGCTACTACAATCAGAGCGAGGCCGGGTCTCACA |
| | | CCCTGCAGTGGATGCATGGCTGCGAGCTGGGGCCCGACGGGCGCTTCCT |
| | | CCGCGGGTATGAACAGTTCGCCTACGACGGCAAGGATTATCTCACCCTG |
| | | AATGAGGACCTGCGCTCCTGGACCGCGGTGGACACGGCGGCTCAGATCT |
| | | CCGAGCAAAAGTCAAATGATGCTTCTGAGGCGGAGCACCAGAGAGCCTA |
| | | CCTGGAAGACACATGCGTGGAGTGGCTCCACAAATACCTGGAGAAGGGG |
| | | AAGGAGACGCTGCTTCACCTGGAGCCCCCAAAGACACACGTGACTCACC |
| | | ACCCCATCTCTGACCATGAGGCCACCCTGAGGTGCTGGGCCCTGGGCTT |
| | | CTACCCTGCGGAGATCACACTGACCTGGCAGCAGGATGGGGAGGGCCAT |
| | | ACCCAGGACACGGAGCTCGTGGAGACCAGGCCTGCAGGGGATGGAACCT |
| | | TCCAGAAGTGGGCAGCTGTGGTGGTGCCTTCTGGAGAGGAGCAGAGATA |
| | | CACGTGCCATGTGCAGCATGAGGGGCTACCCGAGCCCGTCACCCTGAGA |
| | | TGGAAGCCGGCTTCCCAGCCCACCATCCCCATCGTGGGCATCATTGCTG |
| | | GCCTGGTTCTCCTTGGATCTGTGGTCTCTGGAGCTGTGGTTGCTGCTGT |
| | | GATATGGAGGAAGAAGAGCTCAGGTGGAAAAGGAGGGAGCTACTCTAAG |
| | | GCTGAGTGGAGCGACAGTGCCCAGGGGTCTGAGTCTCACAGCTTGTAA |
| | | (SEQ ID NO: 16) |
| Human | AJ293264.1 | ATGGTAGATGGAACCCTCCTTTTACTCCTCTCGGAGGCCCTGGCCCTTA |
| MHC | | CCCAGACCTGGGCGGGCTCCCACTCCTTGAAGTATTTCCACACTTCCGT |
| Class I | | GTCCCGGCCCGGCCGCGGGGAGCCCCGCTTCATCTCTGTGGGCTACGTG |
| antigen, | | GACGACACCCAGTTCGTGCGCTTCGACAACGACGCCGCGAGTCCGAGGA |
| HLA- | | TGGTGCCGCGGGCGCCGTGGATGGAGCAGGAGGGGTCAGAGTATTGGGA |
| E*0101 | | CCGGGAGACACGGAGCGCCAGGGACACCGCACAGATTTTCCGAGTGAAC |
| allele | | CTGCGGACGCTGCGCGGCTACTACAATCAGAGCGAGGCCGGGTCTCACA |
| | | CCCTGCAGTGGATGCATGGCTGCGAGCTGGGGCCCGACAGGCGCTTCCT |
| | | CCGCGGGTATGAACAGTTCGCCTACGACGGCAAGGATTATCTCACCCTG |
| | | AATGAGGACCTGCGCTCCTGGACCGCGGTGGACACGGCGGCTCAGATCT |
| | | CCGAGCAAAAGTCAAATGATGCCTCTGAGGCGGAGCACCAGAGAGCCTA |
| | | CCTGGAAGACACATGCGTGGAGTGGCTCCACAAATACCTGGAGAAGGGG |
| | | AAGGAGACGCTGCTTCACCTGGAGCCCCCAAAGACACACGTGACTCACC |
| | | ACCCCATCTCTGACCATGAGGCCACCCTGAGGTGCTGGGCCCTGGGCTT |
| | | CTACCCTGCGGAGATCACACTGACCTGGCAGCAGGATGGGGAGGGCCAT |
| | | ACCCAGGACACGGAGCTCGTGGAGACCAGGCCTGCAGGGGATGGAACCT |
| | | TCCAGAAGTGGGCAGCTGTGGTGGTGCCTTCTGGAGAGGAGCAGAGATA |
| | | CACGTGCCATGTGCAGCATGAGGGGCTACCCGAGCCCGTCACCCTGAGA |
| | | TGGAAGCCGGCTTCCCAGCCCACCATCCCCATCGTGGGCATCATTGCTG |
| | | GCCTGGTTCTCCTTGGATCTGTGGTCTCTGGAGCTGTGGTTGCTGCTGT |
| | | GATATGGAGGAAGAAGAGCTCAGGTGGAAAAGGAGGGAGCTACTCTAAG |
| | | GCTGAGTGGAGCGACAGTGCCCAGGGGTCTGAGTCTCACAGCTTGTAA |
| | | (SEQ ID NO: 17) |

Exemplary nucleotide sequences encoding human β₂M are provided in Table 6 below. Suitable nucleotide sequences also include nucleotide sequences having about 70%, 75%, 80%, 85%, 90%, 95%, 98%, 99%, or 100% sequence identity to the β₂M coding sequences provided in Table 6 below (i.e., SEQ ID NOs. 18-21).

TABLE 6
Suitable β₂M Coding Sequences

| | GenBank | |
|---|---|---|
| | Accession | |
| Name | Number | Sequence |
| Homo | NM_004048.3 | ATGTCTCGCTCCGTGGCCTTAGCTGTGCTCGCGCTACTCTCTCT |
| Sapiens | | TTCTGGCCTGGAGGCTATCCAGCGTACTCCAAAGATTCAGGTTT |
| beta-2- | | ACTCACGTCATCCAGCAGAGAATGGAAAGTCAAATTTCCTGAAT |
| microglo | | TGCTATGTGTCTGGGTTTCATCCATCCGACATTGAAGTTGACTT |
| bulin | | ACTGAAGAATGGAGAGAGAATTGAAAAAGTGGAGCATTCAGACT |
| (B2M), | | TGTCTTTCAGCAAGGACTGGTCTTTCTATCTCTTGTACTACACT |
| mRNA | | GAATTCACCCCCACTGAAAAAGATGAGTATGCCTGCCGTGTGAA |
| | | CCATGTGACTTTGTCACAGCCCAAGATAGTTAAGTGGGATCGAG |
| | | ACATGTAA (SEQ ID NO: 18) |
| Homo | CR457066.1 | ATGTCTCGCTCCGTGGCCTTAGCTGTGCTCGCGCTACTCTCTCT |
| Sapiens | | TTCTGGCCTGGAGGCTATCCAGCGTACTCCAAAGATTCAGGTTT |
| full open | | ACTCACGTCATCCAGCAGAGAATGGAAAGTCAAATTTCCTGAAT |
| reading | | TGCTATGTGTCTGGGTTTCATCCATCCGACATTGAAGTTGACTT |
| frame | | ACTGAAGAATGGAGAGAGAATTGAAAAAGTGGAGCATTCAGACT |
| cDNA | | TGTCTTTCAGCAAGGACTGGTCTTTCTATCTCTTGTACTACACT |
| clone | | GAATTCACCCCCACTGAAAAAGATGAGTATGCCTGCCGTGTGAA |
| RZPDo8 | | CCATGTGACTTTGTCACAGCCCAAGATAGTTAAGTGGGATCGAG |
| 34B107D | | ACATTTAA (SEQ ID NO: 19) |
| for gene | | |
| B2M | | |
| Homo | BC064910.1 | ATGTCTCGCTCCGTGGCCTTAGCTGTGCTCGCGCTACTCTCTCT |
| Sapiens | | TTCTGGCCTGGAGGCTATCCAGCGTACTCCAAAGATTCAGGTTT |
| beta-2- | | ACTCACGTCATCCAGCAGAGAATGGAAAGTCAAATTTCCTGAAT |
| microglo | | TGCTATGTGTCTGGGTTTCATCCATCCGACATTGAAGTTGACTT |
| bulin, | | ACTGAAGAATGGAGAGAGAATTGAAAAAGTGGAGCATTCAGACT |
| mRNA | | TGTCTTTCAGCAAGGACTGGTCTTTCTATCTCTTGTACTACACT |
| | | GAATTCACCCCCACTGAAAAAGATGAGTATGCCTGCCGTGTGAA |
| | | CCATGTGACTTTGTCACAGCCCAAGATAGTTAAGTGGGATCGAG |
| | | ACATGTAA (SEQ ID NO: 20) |
| Homo | BC032589.1 | ATGTCTCGCTCCGTGGCCTTAGCTGTGCTCGCGCTACTCTCTCT |
| Sapiens | | TTCTGGCCTGGAGGCTATCCAGCGTACTCCAAAGATTCAGGTTT |
| beta-2- | | ACTCACGTCATCCAGCAGAGAATGGAAAGTCAAATTTCCTGAAT |
| microglo | | TGCTATGTGTCTGGGTTTCATCCATCCGACATTGAAGTTGACTT |
| bulin, | | ACTGAAGAATGGAGAGAGAATTGAAAAAGTGGAGCATTCAGACT |
| mRNA | | TGTCTTTCAGCAAGGACTGGTCTTTCTATCTCTTGTACTACACT |
| | | GAATTCACCCCCACTGAAAAAGATGAGTATGCCTGCCGTGTGAA |
| | | CCATGTGACTTTGTCACAGCCCAAGATAGTTAAGTGGGATCGAG |
| | | ACATGTAA (SEQ ID NO: 21) |

The single chain HLA-E/β₂M fusion protein may comprise an HLA-E heavy chain covalently fused to β₂M through a flexible linker. In some embodiments, the flexible linker is a glycine-serine linker, e.g., a G₄S₄ linker (Gornalusse et al., “HLA-E-Expressing Pluripotent Stem Cells Escape Allogenic Responses and Lysis by NK Cells,” Nat. Biotechnol. 35(8):765-772 (2017), which is hereby incorporated by reference in its entirety).

The signal sequence of HLA-G comprises peptide sequences normally presented by HLA-E that inhibit NK cell-dependent lysis through its binding to CD94/NGK2A (Lee et al., “HLA-E is a Major Ligand for the Natural Killer Inhibitory Receptor CD94/NKG2A,” Proc. Natl. Acad. Sci. USA 95:5199-5204 (1998), which is hereby incorporated by reference in its entirety). Thus, in some embodiments, the single chain HLA-E/β₂M fusion protein further comprises an additional glycine-serine linker fused to a non-polymorphic peptide derived from the signal sequence of HLA-G (Gornalusse et al., “HLA-E-Expressing Pluripotent Stem Cells Escape Allogenic Responses and Lysis by NK Cells,” Nat. Biotechnol. 35(8):765-772 (2017), which is hereby incorporated by reference in its entirety).

As described above, the recombinant genetic construct as disclosed herein may alternatively or additionally comprise a nucleotide sequence encoding one or more agents that reduce expression of one or more major histocompatibility class I molecules, in particular one or more HLA-I molecules. In one embodiment, this nucleotide sequence is present in the recombinant genetic construct alone, positioned between the first and second gene sequences. In another embodiment, this nucleotide sequence is present in the recombinant genetic construct in combination with the one or more immune checkpoint protein encoding nucleotide sequences. In this embodiment, the combination of the aforementioned nucleotide sequences is positioned between the first and second gene sequences. The nucleotide sequence encoding the one or more agents that reduce expression of the HLA-I molecules can be positioned 5′ or 3′ to the one or more immune checkpoint protein encoding nucleotide sequences.

The recombinant genetic construct of the present disclosure may comprise a further nucleotide sequence encoding one or more agents that reduce expression of one or more HLA-II molecules. In some embodiments, the nucleotide sequence encoding one or more agents that reduce expression of one or more HLA-II molecules is coupled to the one or more immune checkpoint protein encoding nucleotide sequences and/or to the nucleotide sequence encoding one or more agents that reduce expression of one or more HLA-I molecules.

Suitable agents that reduce expression of the one or more HLA-I and/or HLA-II molecules are described in detail below and include, without limitation, inhibitory oligonucleotide molecules, such as a small hairpin RNA (shRNA), microRNA (miRNA), small interfering RNA (siRNA), and/or antisense oligonucleotide.

The human leukocyte antigen (HLA) system is the major histocompatibility complex (MHC) in humans. Thus for purposes of this disclosure, the terms HLA and MHC are used interchangeably to refer to human genes and proteins of the major histocompatibility complex. In other embodiments, the recombinant genetic construct may comprise a nucleotide sequence encoding one or more agents that reduce expression of one or more non-human, mammalian MHC class I or II molecules, e.g., mouse, rat, pig, horse, monkey MHC class I or II molecules.

Class I MHC proteins (e.g., HLA-I proteins) are heterodimers of two proteins, the α chain, which is a transmembrane protein encoded by the MHC class I genes (chromosome 6 in humans; chromosome 17 in the mouse) and the β2-microglobulin (β2M) chain (chromosomes 15 in humans; chromosomes 2 in the mouse). The α chain folds into three globular domains—α1, α2, and α3. The α1 domain rests upon a unit of β2M. The 3 domain is transmembrane, anchoring the MHC class I molecule to the cell membrane. The MHC class I complex presents foreign peptides/molecules to cells of the immune system. The peptide/molecule being presented is held by the peptide-binding groove, in the central region of the α1/α2 heterodimer of the MHC. Classical MHC class I molecules are highly polymorphic and present epitopes to T cell receptors (TCRs) of CD8⁺ T cells, whereas non-classical MHC class I molecules exhibit limited polymorphism, expression patterns, and presented antigens.

The class I HLA gene cluster in humans encodes the heavy chains of classical (HLA-A, HLA-B, and HLA-C) and non-classical (HLA-E, HLA-F, HLA-G) class I molecules. Thus in one embodiment, the recombinant genetic construct disclosed herein comprises a nucleotide sequence encoding one or more agents that reduce the expression of one or more HLA-I molecules, i.e., HLA-A, HLA-B, HLA-C, HLA-E, HLA-F, HLA-G, or combinations thereof, endogenous to the cell in which the recombinant genetic construct is being expressed. In another embodiment, the recombinant genetic construct disclosed herein comprises a nucleotide sequence encoding an agent that reduces the expression of β2M, thereby reducing the expression of all class I HLAs in the cell.

Class II HLA molecules, i.e., the human form of Class II MHC proteins, are heterodimers of two transmembrane proteins, the α chain and the R chain encoded by the class II genes (HLA-II genes on chromosome 6 in humans; MHC-II genes on chromosome 17 in the mouse). Each of the α chain and the R chain comprise two domains—α1 and α2 and β1 and β2, respectively. The α2 and β2 domains are transmembrane domains of the α chain and β chain, respectively, anchoring the MHC/HLA class II molecule to the membrane. Classical MHC/HLA class II molecules are expressed on the surface of dendritic cells, mononuclear phagocytes, and B-lymphocytes and present peptides to CD4⁺ T cells, whereas non-classical MHC/HLA class II molecules are not exposed on cell membranes, but in internal membranes in lysosomes. Expression of MHC/HLA class II is induced by IFN-γ via the production of MHC class II transactivator (CIITA). Thus, in one embodiment, the nucleotide sequence of the recombinant genetic construct encodes an agent that inhibits CIITA expression, thereby reducing the expression of all class II HLAs in the cell.

HLAs in humans corresponding to MHC class II comprise three gene families, each encoding the α and β chains of class II molecules, respectively. The DR gene family consists of a single DRA gene and up to nine DRB genes (DRB1 to DRB9). The DRA gene encodes an invariable α chain and it binds various β chains encoded by the DRB genes. The DP and DQ families each have one expressed gene for α and β chains and additional unexpressed pseudogenes. The DQA1 and DQB1 gene products associate to form DQ molecules, and the DPA1 and DPB1 products form DP molecules.

As noted above, inhibitory oligonucleotide molecules are suitable agents, encoded by the recombinant genetic construct, for reducing expression of the one or more HLA-I or HLA-II molecules. Exemplary inhibitory oligonucleotide molecules include, without limitation, small hairpin RNAs (shRNA), small interfering RNAs (siRNA), microRNAs (miRNA), and/or an antisense oligonucleotides.

siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of any one of the HLA-I and/or HLA-II mRNAs, β2M mRNA (e.g., SEQ ID Nos: 18-21), and/or CIITA mRNA (SEQ ID NO: 22-23). The sequences of various HLA-I (HLA-A, HLA-B, HLA-C) mRNAs and HLA-II (HLA-E, HLA-F, HLA-G) mRNAs, are readily known in the art and accessible to one of skill in the art for purposes of designing siRNA and shRNA oligonucleotides. siRNA molecules are typically designed to target a region of the mRNA target approximately 50-100 nucleotides downstream from the start codon. Methods and online tools for designing suitable siRNA sequences based on the target mRNA sequences are readily available in the art (see e.g., Reynolds et al., “Rational siRNA Design for RNA Interference,” Nat. Biotech. 2:326-330 (2004); Chalk et al., “Improved and Automated Prediction of Effective siRNA,” Biochem. Biophys. Res. Comm. 319(1): 264-274 (2004); Zhang et al., “Weak Base Pairing in Both Seed and 3′ Regions Reduces RNAi Off-targets and Enhances si/shRNA Designs,” Nucleic Acids Res. 42(19):12169-76 (2014), which are hereby incorporated by reference in their entirety). Upon introduction into a cell, the siRNA complex triggers the endogenous RNA interference (RNAi) pathway, resulting in the cleavage and degradation of the target mRNA molecule. Various improvements of siRNA compositions, such as the incorporation of modified nucleosides or motifs into one or both strands of the siRNA molecule to enhance stability, specificity, and efficacy, have been described and are suitable for use in accordance with this aspect of the invention (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety). Methods of constructing DNA-vectors for siRNA expression in mammalian cells are known in the art, see e.g., Sui et al., “A DNA Vector-Based RNAi Technology to Suppress Gene Expression in Mammalian Cells,” Proc. Nat'l Acad. Sci. USA 99(8):5515-5520 (2002), which is hereby incorporated by reference.

TABLE 7
Human CIITA mRNA Sequences

| | Gene | |
|---|---|---|
| Name | Accession | Sequence (SEQ ID NOs: 22-23) |
| H. sapiens | X74301.1 | tgatgaggct gtgtgcttct gagctgggca tccgaaggca |
| mRNA for | | tccttgggga agctgagggc acgaggaggg gctgccagac |
| MHC class II | | tccgggagct gctgcctggc tgggattcct acacaatgcg |
| transactivator | | ttgcctggct ccacgccctg ctgggtccta cctgtcagag |
| | | ccccaaggca gctcacagtg tgccaccatg gagttggggc |
| | | ccctagaagg tggctacctg gagcttctta acagcgatgc |
| | | tgaccccctg tgcctctacc acttctatga ccagatggac |
| | | ctggctggag aagaagagat tgagctctac tcagaacccg |
| | | acacagacac catcaactgc gaccagttca gcaggctgtt |
| | | gtgtgacatg gaaggtgatg aagagaccag ggaggcttat |
| | | gccaatatcg cggaactgga ccagtatgtc ttccaggact |
| | | cccagctgga gggcctgagc aaggacattt tcaagcacat |
| | | aggaccagat gaagtgatcg gtgagagtat ggagatgcca |
| | | gcagaagttg ggcagaaaag tcagaaaaga cccttcccag |
| | | aggagcttcc ggcagacctg aagcactgga agccagctga |
| | | gccccccact gtggtgactg gcagtctcct agtgggacca |
| | | gtgagcgact gctccaccct gccctgcctg ccactgcctg |
| | | cgctgttcaa ccaggagcca gcctccggcc agatgcgcct |
| | | ggagaaaacc gaccagattc ccatgccttt ctccagttcc |
| | | tcgttgagct gcctgaatct ccctgaggga cccatccagt |
| | | ttgtccccac catctccact ctgccccatg ggctctggca |
| | | aatctctgag gctggaacag gggtctccag tatattcatc |
| | | taccatggtg aggtgcccca ggccagccaa gtaccccctc |
| | | ccagtggatt cactgtccac ggcctcccaa catctccaga |
| | | ccggccaggc tccaccagcc ccttcgctcc atcagccact |
| | | gacctgccca gcatgcctga acctgccctg acctcccgag |
| | | caaacatgac agagcacaag acgtccccca cccaatgccc |
| | | ggcagctgga gaggtctcca acaagcttcc aaaatggcct |
| | | gagccggtgg agcagttcta ccgctcactg caggacacgt |
| | | atggtgccga gcccgcaggc ccggatggca tcctagtgga |
| | | ggtggatctg gtgcaggcca ggctggagag gagcagcagc |
| | | aagagcctgg agcgggaact ggccaccccg gactgggcag |
| | | aacggcagct ggcccaagga ggcctggctg aggtgctgtt |
| | | ggctgccaag gagcaccggc ggccgcgtga gacacgagtg |
| | | attgctgtgc tgggcaaagc tggtcagggc aagagctatt |
| | | gggctggggc agtgagccgg gcctgggctt gtggccggct |
| | | tccccagtac gactttgtct tctctgtccc ctgccattgc |
| | | ttgaaccgtc cgggggatgc ctatggcctg caggatctgc |
| | | tcttctccct gggcccacag ccactcgtgg cggccgatga |
| | | ggttttcagc cacatcttga agagacctga ccgcgttctg |
| | | ctcatcctag acgccttcga ggagctggaa gcgcaagatg |
| | | gcttcctgca cagcacgtgc ggaccggcac cggcggagcc |
| | | ctgctccctc cgggggctgc tggccggcct tttccagaag |
| | | aagctgctcc gaggttgcac cctcctcctc acagcccggc |
| | | cccggggccg cctggtccag agcctgagca aggccgacgc |
| | | cctatttgag ctgtccggct tctccatgga gcaggcccag |
| | | gcatacgtga tgcgctactt tgagagctca gggatgacag |
| | | agcaccaaga cagagccctg acgctcctcc gggaccggcc |
| | | acttcttctc agtcacagcc acagccctac tttgtgccgg |
| | | gcagtgtgcc agctctcaga ggccctgctg gagcttgggg |
| | | aggacgccaa gctgccctcc acgctcacgg gactctatgt |
| | | cggcctgctg ggccgtgcag ccctcgacag cccccccggg |
| | | gccctggcag agctggccaa gctggcctgg gagctgggcc |
| | | gcagacatca aagtacccta caggaggacc agttcccatc |
| | | cgcagacgtg aggacctggg cgatggccaa aggcttagtc |
| | | caacacccac cgcgggccgc agagtccgag ctggccttcc |
| | | ccagcttcct cctgcaatgc ttcctggggg ccctgtggct |
| | | ggctctgagt ggcgaaatca aggacaagga gctcccgcag |
| | | tacctagcat tgaccccaag gaagaagagg ccctatgaca |
| | | actggctgga gggcgtgcca cgctttctgg ctgggctgat |
| | | cttccagcct cccgcccgct gcctgggagc cctactcggg |
| | | ccatcggcgg ctgcctcggt ggacaggaag cagaaggtgc |
| | | ttgcgaggta cctgaagcgg ctgcagccgg ggacactgcg |
| | | ggcgcggcag ctgcttgagc tgctgcactg cgcccacgag |
| | | gccgaggagg ctggaatttg gcagcacgtg gtacaggagc |
| | | tccccggccg cctctctttt ctgggcaccc gcctcacgcc |
| | | tcctgatgca catgtactgg gcaaggcctt ggaggcggcg |
| | | ggccaagact tctccctgga cctccgcagc actggcattt |
| | | gcccctctgg attggggagc ctcgtgggac tcagctgtgt |
| | | cacccgtttc agggctgcct tgagcgacac ggtggcgctg |
| | | tgggagtccc tgcggcagca tggggagacc aagctacttc |
| | | aggcagcaga ggagaagttc accatcgagc ctttcaaagc |
| | | caagtccctg aaggatgtgg aagacctggg aaagcttgtg |
| | | cagactcaga ggacgagaag ttcctcggaa gacacagctg |
| | | gggagctccc tgctgttcgg gacctaaaga aactggagtt |
| | | tgcgctgggc cctgtctcag gcccccaggc tttccccaaa |
| | | ctggtgcgga tcctcacggc cttttcctcc ctgcagcatc |
| | | tggacctgga tgcgctgagt gagaacaaga tcggggacga |
| | | gggtgtctcg cagctctcag ccaccttccc ccagctgaag |
| | | tccttggaaa ccctcaatct gtcccagaac aacatcactg |
| | | acctgggtgc ctacaaactc gccgaggccc tgccttcgct |
| | | cgctgcatcc ctgctcaggc taagcttgta caataactgc |
| | | atctgcgacg tgggagccga gagcttggct cgtgtgcttc |
| | | cggacatggt gtccctccgg gtgatggacg tccagtacaa |
| | | caagttcacg gctgccgggg cccagcagct cgctgccagc |
| | | cttcggaggt gtcctcatgt ggagacgctg gcgatgtgga |
| | | cgcccaccat cccattcagt gtccaggaac acctgcaaca |
| | | acaggattca cggatcagcc tgagatgatc ccagctgtgc |
| | | tctggacagg catgttctct gaggacacta accacgctgg |
| | | accttgaact gggtacttgt ggacacagct cttctccagg |
| | | ctgtatccca tgaggcctca gcatcctggc acccggcccc |
| | | tgctggttca gggttggccc ctgcccggct gcggaatgaa |
| | | ccacatcttg ctctgctgac agacacaggc ccggctccag |
| | | gctcctttag cgcccagttg ggtggatgcc tggtggcagc |
| | | tgcggtccac ccaggagccc cgaggccttc tctgaaggac |
| | | attgcggaca gccacggcca ggccagaggg agtgacagag |
| | | gcagccccat tctgcctgcc caggcccctg ccaccctggg |
| | | gagaaagtac ttcttttttt ttatttttag acagagtctc |
| | | actgttgccc aggctggcgt gcagtggtgc gatctgggtt |
| | | cactgcaacc tccgcctctt gggttcaagc gattcttctg |
| | | cttcagcctc ccgagtagct gggactacag gcacccacca |
| | | tcatgtctgg ctaatttttc atttttagta gagacagggt |
| | | tttgccatgt tggccaggct ggtctcaaac tcttgacctc |
| | | aggtgatcca cccacctcag cctcccaaag tgctggggat |
| | | tacaagcgtg agccactgca ccgggccaca gagaaagtac |
| | | ttctccaccc tgctctccga ccagacacct tgacagggca |
| | | caccgggcac tcagaagaca ctgatgggca acccccagcc |
| | | tgctaattcc ccagattgca acaggctggg cttcagtggc |
| | | aggctgcttt tgtctatggg actcaatgca ctgacattgt |
| | | tggccaaagc caaagctagg cctggccaga tgcaccaggc |
| | | ccttagcagg gaaacagcta atgggacact aatggggcgg |
| | | tgagagggga acagactgga agcacagctt catttcctgt |
| | | gtcttttttc actacattat aaatgtctct ttaatgtcac |
| | | aaaaaaaaaa aaaaaaaaaa aaa (SEQ ID NO: 22) |
| Homo | AF410154.1 | cctcccaact ggtgactggt tagtgatgag gctgtgtgct |
| sapiens | | tctgagctgg gcatccgaag gcatccttgg ggaagctgag |
| MHC2TA | | ggcacgagga ggggctgcca gactccggga gctgctgcct |
| mRNA, | | ggctgggatt cctacacaat gcgttgcctg gctccacgcc |
| altern. | | ctgctgggtc ctacctgtca gagccccaag gcagctcaca |
| spliced | | gtgtgccacc atggagttgg ggcccctaga aggtggctac |
| | | ctggagcttc ttaacagcga tgctgacccc ctgtgcctct |
| | | accacttcta tgaccagatg gacctggctg gagaagaaga |
| | | gattgagctc tactcagaac ccgacacaga caccatcaac |
| | | tgcgaccagt tcagcaggct gttgtgtgac atggaaggtg |
| | | atgaagagac cagggaggct tatgccaata tcgcggaact |
| | | ggaccagtat gtcttccagg actcccagct ggagggcctg |
| | | agcaaggaca ttttcaagca cataggacca gatgaagtga |
| | | tcggtgagag tatggagatg ccagcagaag ttgggcagaa |
| | | aagtcagaaa agacccttcc cagaggagct tccggcagac |
| | | ctgaagcact ggaagccagc tgagcccccc actgtggtga |
| | | ctggcagtct cctagtggga ccagtgagcg actgctccac |
| | | cctgccctgc ctgccactgc ctgcgctgtt caaccaggag |
| | | ccagcctccg gccagatgcg cctggagaaa accgaccaga |
| | | ttcccatgcc tttctccagt tcctcgttga gctgcctgaa |
| | | tctccctgag ggacccatcc agtttgtccc caccatctcc |
| | | actctgcccc atgggctctg gcaaatctct gaggctggaa |
| | | caggggtctc cagtatattc atctaccatg gtgaggtgcc |
| | | ccaggccagc caagtacccc ctcccagtgg attcactgtc |
| | | cacggcctcc caacatctcc agaccggcca ggctccacca |
| | | gccccttcgc tccatcagcc actgacctgc ccagcatgcc |
| | | tgaacctgcc ctgacctccc gagcaaacat gacagagcac |
| | | aagacgtccc ccacccaatg cccggcagct ggagaggtct |
| | | ccaacaagct tccaaaatgg cctgagccgg tggagcagtt |
| | | ctaccgctca ctgcaggaca cgtatggtgc cgagcccgca |
| | | ggcccggatg gcatcctagt ggaggtggat ctggtgcagg |
| | | ccaggctgga gaggagcagc agcaagagcc tggagcggga |
| | | actggccacc ccggactggg cagaacggca gctggcccaa |
| | | ggaggcctgg ctgaggtgct gttggctgcc aaggagcacc |
| | | ggcggccgcg tgagacacga gtgattgctg tgctgggcaa |
| | | agctggtcag ggcaagagct attgggctgg ggcagtgagc |
| | | cgggcctggg cttgtggccg gcttccccag tacgactttg |
| | | tcttctctgt cccctgccat tgcttgaacc gtccggggga |
| | | tgcctatggc ctgcaggatc tgctcttctc cctgggccca |
| | | cagccactcg tggcggccga tgaggttttc agccacatct |
| | | tgaagagacc tgaccgcgtt ctgctcatcc tagacgcctt |
| | | cgaggagctg gaagcgcaag atggcttcct gcacagcacg |
| | | tgcggaccgg caccggcgga gccctgctcc ctccgggggc |
| | | tgctggccgg ccttttccag aagaagctgc tccgaggttg |
| | | caccctcctc ctcacagccc ggccccgggg ccgcctggtc |
| | | cagagcctga gcaaggccga cgccctattt gagctgtccg |
| | | gcttctccat ggagcaggcc caggcatacg tgatgcgcta |
| | | ctttgagagc tcagggatga cagagcacca agacagagcc |
| | | ctgacgctcc tccgggaccg gccacttctt ctcagtcaca |
| | | gccacagccc tactttgtgc cgggcagtgt gccagctctc |
| | | agaggccctg ctggagcttg gggaggacgc caagctgccc |
| | | tccacgctca cgggactcta tgtcggcctg ctgggccgtg |
| | | cagccctcga cagccccccc ggggccctgg cagagctggc |
| | | caagctggcc tgggagctgg gccgcagaca tcaaagtacc |
| | | ctacaggagg accagttccc atccgcagac gtgaggacct |
| | | gggcgatggc caaaggctta gtccaacacc caccgcgggc |
| | | cgcagagtcc gagctggcct tccccagctt cctcctgcaa |
| | | tgcttcctgg gggccctgtg gctggctctg agtggcgaaa |
| | | tcaaggacaa ggagctcccg cagtacctag cattgacccc |
| | | aaggaagaag aggccctatg acaactggct ggagggcgtg |
| | | ccacgctttc tggctgggct gatcttccag cctcccgccc |
| | | gctgcctggg agccctactc gggccatcgg cggctgcctc |
| | | ggtggacagg aagcagaagg tgcttgcgag gtacctgaag |
| | | cggctgcagc cggggacact gcgggcgcgg cagctgcttg |
| | | agctgctgca ctgcgcccac gaggccgagg aggctggaat |
| | | ttggcagcac gtggtacagg agctccccgg ccgcctctct |
| | | tttctgggca cccgcctcac gcctcctgat gcacatgtac |
| | | tgggcaaggc cttggaggcg gcgggccaag acttctccct |
| | | ggacctccgc agcactggca tttgcccctc tggattgggg |
| | | agcctcgtgg gactcagctg tgtcacccgt ttcaggtggg |
| | | gtgaggggct tggaagagac atccttgtgt tgggcattaa |
| | | ctgcggtctt ggtgccaagc ccagtgctct gtggggtcct |
| | | tttagtatgc agagcagccg ggtggggcag aatggattct |
| | | ctccattttt aagatgagga tgttgaggct cagagagggg |
| | | cagccacttg ccacacagca agtgagaggc aatggcattc |
| | | tcccagtcaa tatttgaagg cccgccatgt gccagtcact |
| | | ggggtatgtc tagaatctga gactgacctg ggctcaaatt |
| | | tgttttattc tttccacccc ctgagcacgc caccgttttc |
| | | ttatgctaag agtaaagcca tggcctcccc ttggactctc |
| | | tgcctccatt ctctcctctt ccactccatt ttgtattcag |
| | | caaccagacc aatcttctca gaacttgaat ctgattgtat |
| | | cccatccctg cttacaatcc ttcagggaca ctccaccact |
| | | gtcaggatga aggctaaatt tcttaatttg gtttcattaa |
| | | gtcggtctgc aatctgcttg agcatttcag cttaatcgcc |
| | | agaggattgc ttccatattt ccccctaaac atactttacc |
| | | caagctgtaa ggtcctacat aattgtgcca ataatttagc |
| | | agtgagcttc ctggtagccg aagcaaaaag ggaaagaaaa |
| | | ccactgtgtg agttgtgaga aagtaggaat caataaaggc |
| | | tggagtggtc gctgccttga gcgacacggt ggcgatggaa |
| | | ggctttctgg gaaaggtaga ggttgagcta aggaaagaaa |
| | | gtattttaat aggtaggagg acccttcatg gagctgccct |
| | | tccattaagg tctagcctgg tcaccgtgcc tgggtctgag |
| | | gccctccctc cacaggctgt gggagtccct gcggcagcat |
| | | ggggagacca agctacttca ggcagcagag gagaagttca |
| | | ccatcgagcc tttcaaagcc aagtccctga aggatgtgga |
| | | agacctggga aagcttgtgc agactcagag gacgagaagt |
| | | tcctcggaag acacagctgg ggagctccct gctgttcggg |
| | | acctaaagaa actggagttt gcgctgggcc ctgtctcagg |
| | | cccccaggct ttccccaaac tggtgcggat cctcacggcc |
| | | ttttcctccc tgcagcatct ggacctggat gcgctgagtg |
| | | agaacaagat cggggacgag ggtgtctcgc agctctcagc |
| | | caccttcccc cagctgaagt ccttggaaac cctcaatctg |
| | | tcccagaaca acatcactga cctgggtgcc tacaaactcg |
| | | ccgaggccct gccttcgctc gctgcatccc tgctcaggct |
| | | aagcttgtac aataactgca tctgcgacgt gggagccgag |
| | | agcttggctc gtgtgcttcc ggacatggtg tccctccggg |
| | | tgatggacgt ccagtacaac aagttcacgg ctgccggggc |
| | | ccagcagctc gctgccagcc ttcggaggtg tcctcatgtg |
| | | gagacgctgg cgatgtggac gcccaccatc ccattcagtg |
| | | tccaggaaca cctgcaacaa caggattcac ggatcagcct |
| | | gagatgatcc cagctgtgct ctggacaggc atgttctctg |
| | | aggacactaa ccacgctgga ccttgaactg ggtacttgtg |
| | | gacacagctc ttctccaggc tgtatcccat gagcctcagc |
| | | atcctggcac ccggcccctg ctggttcagg gttggcccct |
| | | gcccggctgc ggaatgaacc acatcttgct ctgctgacag |
| | | acacaggccc ggctccaggc tcctttagcg cccagttggg |
| | | tggatgcctg gtggcagctg cggtccaccc aggagccccg |
| | | aggccttctc tgaaggacat tgcggacagc cacggccagg |
| | | ccagagggag tgacagaggc agccccattc tgcctgccca |
| | | ggcccctgcc accctgggga gaaagtactt cttttttttt |
| | | atttttagac agggtctcac tgttgcccag gctggcgtgc |
| | | agtggtgcga tctgggttca ctgcaacctc cgcctcttgg |
| | | gttcaagcga ttcttctgct tcagcctccc gagtagctgg |
| | | gactacaggc acccaccatc atgtctggct aatttttcat |
| | | ttttggtaga gacagggttt tgccgtgttg gccgggctgg |
| | | tctcgaactc ttgacctcgg gtgatccacc cacctcagcc |
| | | tcccaaagtg ctgggattac aagcgtgagc cactgcaccg |
| | | ggccacagag aaagtacttc tccaccctgc tctccgacca |
| | | gacaccttga cagggcacac cgggcactca gaagacactg |
| | | atgggcaacc cccagcctgc taattcccca gattgcaaca |
| | | ggctgggctt cagtggcagc tgcttttgtc tatgggactc |
| | | aatgcactga cattgttggc caaagccaaa gctaggcctg |
| | | gccagatgca ccagccctta gcagggaaac agctaatggg |
| | | acactaatgg ggcggtgaga ggggaacaga ctggaa (SEQ |
| | | ID NO: 23) |

Short or small hairpin RNA (shRNA) molecules are similar to siRNA molecules in function, but comprise longer RNA sequences that make a tight hairpin turn. shRNA is cleaved by cellular machinery into siRNA and gene expression is silenced via the cellular RNA interference pathway. Methods and tools for designing suitable shRNA sequences based on the target mRNA sequences (e.g., β2M, CIITA, and other HLA-I and HLA-II mRNA sequences) are readily available in the art (see e.g., Taxman et al., “Criteria for Effective Design, Constructions, and Gene Knockdown shRNA Vectors,” BMC Biotech. 6:7 (2006) and Taxman et al., “Short Hairpin RNA (shRNA): Design, Delivery, and Assessment of Gene Knockdown,” Meth. Mol. Biol. 629: 139-156 (2010), which are hereby incorporated by reference in their entirety). Methods of constructing DNA-vectors for shRNA expression and gene silencing in mammalian cells is described herein and are known in the art, see e.g., Cheng and Chang, “Construction of Simple and Efficient DNA Vector-based Short Hairpin RNA Expression Systems for Specific Gene Silencing in Mammalian Cells,” Methods Mol. Biol. 408:223-41 (2007), which is hereby incorporated by reference in its entirety.

Other suitable agents that can be encoded by the recombinant construct disclosed herein for purposes of inhibiting HLA-I or HLA-II molecules include microRNAs (miRNAs). miRNAs are small, regulatory, noncoding RNA molecules that control the expression of their target mRNAs predominantly by binding to the 3′ untranslated region (UTR). A single UTR may have binding sites for many miRNAs or multiple sites for a single miRNA, suggesting a complex post-transcriptional control of gene expression exerted by these regulatory RNAs (Shulka et al., “MicroRNAs: Processing, Maturation, Target Recognition and Regulatory Functions,” Mol. Cell. Pharmacol. 3(3):83-92 (2011), which is hereby incorporated by reference in its entirety). Mature miRNA are initially expressed as primary transcripts known as a pri-miRNAs which are processed, in the cell nucleus, to 70-nucleotide stem-loop structures called pre-miRNAs by the microprocessor complex. The dsRNA portion of the pre-miRNA is bound and cleaved by Dicer to produce a mature 22 bp double-stranded miRNA molecule that can be integrated into the RISC complex; thus, miRNA and siRNA share the same cellular machinery downstream of their initial processing.

microRNAs known to inhibit the expression of MHC class I molecules are known in the art and suitable for incorporation into the recombinant genetic construct described herein. For example, miR-148a is known to modulate expression of HLA-C(O'Huigin et al., “The Molecular Origin and Consequences of Escape from miRNA Regulation by HLA-C Alleles,” Am. J. Hum. Genet. 89(3):424-431 (2011), which is hereby incorporated by reference in its entirety); miR-148 and miR-152 down-regulate HLA-G expression (Manaster et al., “miRNA-mediated Control of HLA-G Expression and Function,” PLoS ONE 7(3): e33395 (2012), which is hereby incorporated by reference in its entirety); miR-9 modulates expression of β2-microglobulin, HLA-B, and other class I MHC molecules (Gao et al., “MiR-9 Modulates the Expression of Interferon-Regulated Genes and MHC Class I Molecules in Human Nasopharyngeal Carcinoma Cells,” Biochem. Biophys. Res. Commun. 4313:610-616 (2013), which is hereby incorporated by reference in its entirety); miR-181a modulates expression of HLA-A (Liu et al., “Altered Expression Profiles of microRNAs in a Stable Hepatitis B Virus-Expressing Cell Line,” Chin. Med J. 1221:10-14 (2009), which is hereby incorporated by reference in its entirety). Methods of constructing DNA-vectors for miRNA expression and gene silencing in mammalian cells are known in the art, see e.g., Yang N., “An Overview of Viral and Non-Viral Delivery Systems for microRNA,” Int. J. Pharm. Investig. 5(4):179-181 (2015).

Other suitable agents that can be encoded by the recombinant construct disclosed herein for purposes of inhibiting HLA-I or HLA-II molecules include antisense nucleotides. The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 7,179,796 to Cowsert et al., which are hereby incorporated by reference in their entirety). Antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule, such as an mRNA molecule (see e.g., Weintraub, H. M., “Antisense DNA and RNA,” Scientific Am. 262:40-46 (1990), which is hereby incorporated by reference in its entirety). The antisense nucleic acid molecule hybridizes to its corresponding target nucleic acid molecule, such as any of the HLA-I or HLA-II mRNAs, β2M mRNA, or CIITA mRNA, to form a double-stranded molecule, which interferes with translation of the mRNA, as the cell will not translate a double-stranded mRNA. Antisense nucleic acids used in the methods of the present invention are typically at least 10-15 nucleotides in length, for example, at least 10, 11, 12, 13, 14, 15, 16, 17, 18, 19, 20, 25, 30, 35, 40, 45, 50, 55, 60, 65, 70, 75, or greater than 75 nucleotides in length. The antisense nucleic acid can also be as long as its target nucleic acid with which it is intended to form an inhibitory duplex.

In some embodiments, the nucleotide sequence encoding one or more agents that reduce expression of one or more HLA-I or HLA-II molecules encodes a plurality (e.g., at least 2, 3, 4, 5, 6, 7, 8, 9, 10, 11, 12, 13, 14, 15, or more) of RNA molecules.

In some embodiments, the one or more agents that encoded by the recombinant genetic constructs as disclosed herein that inhibit one or more HLA-I and/or HLA-II molecules include a CRISPR/Cas9 system or zinc-finger nuclease.

CRISPR/CRISPR-associated (Cas) systems use single guide RNAs to target and cleave DNA elements in a sequence-specific manner. CRISPR/Cas systems are well known in the art and include, e.g., the type II CRISPR system from Streptococcus pyogenes (Qi et al, “Repurposing CRISPR as an RNA-Guided Platform for Sequence-Specific Control of Gene Expression,” Cell 152(5):1173-1183 (2013), which is hereby incorporated by reference in its entirety). The Streptococcus pyogenes type II CRISPR system includes a single gene encoding the Cas9 protein and two RNAs, a mature CRISPR RNA (crRNA), and a partially complementary trans-acting RNA (tracrRNA). Maturation of the crRNA requires tracrRNA and RNase II. However, this requirement can be bypassed by using an engineered small guide RNA (sgRNA) containing a designed hairpin that mimics the tracrRNA-crRNA complex. Base pairing between the sgRNA and target DNA causes double-strand breaks (DSBs) due to the endonuclease activity of Cas9. Binding specificity is determined by both sgRNA-DNA base pairing and a short DNA motif (protospacer adjacent motif (PAM) sequence: NGG) juxtaposed to the DNA complementary region.

In some embodiments, the CRISPR/Cas 9 system encoded by the recombinant genetic construct comprises a Cas9 protein and a sgRNA.

The Cas9 protein may comprise a wild-type Cas9 protein or a nuclease-deficient Cas9 protein. Binding of wild-type Cas9 to the sgRNA forms a protein-RNA complex that mediates cleavage of a target DNA by the cas9 nuclease. Binding of nuclease deficient Cas9 to the sgRNA forms a protein-RNA complex that mediates transcriptional regulation of a target DNA by the nuclease deficient Cas9 (Qi et al, “Repurposing CRISPR as an RNA-Guided Platform for Sequence-Specific Control of Gene Expression,” Cell 152(5):1173-1183 (2013); Maeder et al., “CRISPR RNA-Guided Activation of Endogenous Human Genes,” Nat. Methods 10(10):977-999 (2013); and Gilbert et al., “CRISPR-Mediated Modular RNA-Guided Regulation of Transcription in Eukaryotes,” Cell 154(2):442-451 (2013), which are hereby incorporated by reference in their entirety).

The sgRNA comprises a region complementary to a specific DNA sequence (e.g., a region of the HLA-I or HLA-II gene), a hairpin for Cas9 binding, and/or a transcription terminator (Qi et al, “Repurposing CRISPR as an RNA-Guided Platform for Sequence-Specific Control of Gene Expression,” Cell 152(5):1173-1183 (2013), which is hereby incorporated by reference in its entirety). Methods of designing sgRNA for the purposes of targeting specific gene sequence are well known in the art and are described in more detail in, e.g., WO2015/089364, WO2014/191521 and WO2015/065964, which are hereby incorporated by reference in their entirety).

In another embodiment, the one or more agents encoded by the recombinant genetic construct disclosed herein for purposes of inhibiting HLA-I or HLA-II molecules is a zinc finger nuclease. Zinc finger nucleases (ZFNs) are synthetic enzymes comprising three (or more) zinc finger domains linked together to create an artificial DNA-binding protein that binds >9 bp of DNA. In order to cut DNA, the zinc finger domains are fused to one half of the FokI nuclease domain such that when two ZFNs bind the two unique 9 bp sites, separated by a suitable spacer, they can cut within the spacer to make a DSB. Methods of designing zinc finger nucleases to recognize a desired target are well known in the art and are described in more detail in, e.g., U.S. Pat. No. 7,163,824 to Cox III; U.S. Patent Application Publication No. 2017/0327795 to Kim et al.; and Harrison et al., “A Beginner's Guide to Gene Editing,” Exp. Physiol. 103(4):439-448 (2018), which are hereby incorporated by reference in their entirety).

In some embodiments, the one or more agents that reduce expression of one or more endogenous HLA-I and/or HLA-II molecules reduce expression of all HLA-I and/or HLA-II molecules. In some embodiments, the one or more agents are capable of reducing the expression of the one or more HLA-I and/or HLA-II molecules on the surface of a cell by 5%, 6%, 7%, 8%, 9%, 10%, 20%, 30%0, 40%, 50%, 60%, 70%, 80%, 90%, 95%, 99%, 99.5%, 99.9% or 100% relative to the wildtype level of expression.

The recombinant genetic constructs described herein further comprise first and second “gene sequences” also referred to herein as “homology arms”. These gene sequences, which are expressed in a cell-type specific manner, direct insertion of the recombinant construct into a gene of interest (i.e., a target gene) within a population of cells by, for example, homologous recombination. Thus, the recombinant genetic construct comprises a first gene sequence expressed in a cell-type specific manner that is located 5′ to the one or more immune checkpoint protein encoding nucleotide sequences and/or the one or more nucleotides sequences encoding agent(s) for reducing expression of HLA-I and/or HLA-II molecules, and a second gene sequence that is expressed in the same cell-type specific manner as the first gene sequence. The second gene sequence is located 3′ to the one or more immune checkpoint protein encoding nucleotide sequences and/or the one or more nucleotides sequences encoding agent(s) for reducing expression of HLA-I and/or HLA-II molecules.

The first and second gene sequence(s) of the recombinant genetic construct described herein are nucleotide sequences that are the same as or closely homologous (i.e., sharing significant sequence identity) to the nucleotide sequence of particular regions of the target gene, i.e., the gene in which the recombinant genetic construct will be inserted into. Preferably, the first and second gene sequences of the recombinant construct are the same as or similar to the target gene sequence (e.g., the same as the sense strand of the target gene) immediately upstream and downstream of an insertion cleavage site.

In some embodiments, the percent identity between the first gene sequence located at the 5′ end of the recombinant construct (i.e., a 5′ homology arm) and the corresponding sequence of target gene (e.g., sense strand) is at least about 70%, 75%, 80%, 85%, 90%, 95%, 98%, 99%, or 100%. In some embodiments, the percent identity between the second gene sequence located at the 3′ end of the recombinant construct (i.e., a 3′ homology arm) and the corresponding sequence of the target gene (e.g., sense strand) is at least about 70%, 75%, 80%, 85%, 90%, 95%, 98%, 99%, or 100%.

In some embodiments, the first and second gene sequences (e.g., the 5′ and 3′ homology arms) are more than about 30 nucleotide residues in length, for example more than about any of 50 nucleotide residues, 100 nucleotide residues, 200 nucleotide residues, 300 nucleotide residues, 500 nucleotide residues, 800 nucleotide residues, 1,000 nucleotide residues, 1,500 nucleotide residues, 2,000 nucleotide residues, and 5,000 nucleotide residues in length.

The recombinant genetic construct as disclosed herein may be circular or linear. When the recombinant genetic construct is linear, the first and second gene sequences (e.g., the 5′ and 3′ homology arms) are proximal to the 5′ and 3′ ends of the linear nucleic acid, respectively, i.e., about 200 bp away from the 5′ and 3′ ends of the linear nucleic acid. In some embodiments, the first gene sequence (e.g., the 5′ homology arm) is about any of 1, 2, 5, 10, 15, 20, 25, 30, 35, 40, 45, 50, 55, 60, 70, 80, 90, 100, 120, 140, 160, 180, or 200 nucleotide residues away from the 5′ end of the linear DNA. In some embodiments, the second gene sequence (e.g., the 3′ homology arm) is about any of 1, 2, 5, 10, 15, 20, 25, 30, 35, 40, 45, 50, 55, 60, 70, 80, 90, 100, 120, 140, 160, 180, or 200 nucleotide residues away from the 3′ end of the linear DNA.

The first and second gene sequences of the recombinant genetic construct are designed to mimic sequences of a “target gene” to facilitate insertion of the construct into the target gene. In accordance with various aspects of the present disclosure, the “target gene” is a gene that is expressed in a cell-type specific manner. In some embodiments, the “target gene” is a gene that is selectively and/or restrictively expressed in a terminally differentiated cell. A “terminally differentiated cell” refers to a specialized cell that has acquired and is committed to specialized functions, and has irreversibly lost its ability to divide and proliferate.

In some embodiments, the target gene is a gene that is expressed in a terminally differentiated cell of the central nervous system. Exemplary terminally differentiated brain cells include, without limitation, oligodendrocytes, astrocytes, and neurons, including cholinergic neurons, medium spiny neurons and interneurons, and dopaminergic neurons. Exemplary terminally differentiated brain cells and gene targets selectively expressed in these cells are identified in Table 8 and discussed in more detail below.

TABLE 8
Exemplary CNS Cells and Gene Targets Selectively Expressed Therein

| Terminally | | | |
|---|---|---|---|
| Differentiated Cell | Cell Specific Gene | | |
| Type | Target | Organism | Gene ID: |
| Oligodendrocyte | SOX10 | Human | 6663 |
| | | Mouse | 20665 |
| | MYRF | Human | 745 |
| | | Mouse | 225908 |
| | MAG | Human | 4099 |
| | | Mouse | 17136 |
| | MBP | Human | 4155 |
| | | Mouse | 17196 |
| Astrocyte | GFAP | Human | 2670 |
| | | Mouse | 14580 |
| | AQP4 | Human | 361 |
| | | Mouse | 11829 |
| Neurons | SYN1 | Human | 6853 |
| | | Mouse | 20964 |
| | MAP2 | Human | 4133 |
| | | Mouse | 17756 |
| | ELAV4 | Human | 1996 |
| | | Mouse | 15572 |
| Dopaminergic Neurons | TH (tyrosine | Human | 7054 |
| | hydroxylase) | Mouse | 21823 |
| | DDC (DOPA | Human | 1644 |
| | decarboxylase) | Mouse | 13195 |
| Cholinergic Neurons | CHAT (Choline O- | Human | 1103 |
| | acetyltransferase) | Mouse | 12647 |
| Medium spiny | GAD65 | Human | 2572 |
| neurons/interneurons | | Mouse | 14417 |
| | GAD67 | Human | 2571 |
| | | Mouse | 14415 |
| Glutaminergic Neurons | SLC17A6 | Human | 57084 |
| | | Mouse | 140919 |
| | SLC17A7 | Human | 57030 |
| | | Mouse | 72961 |

In one embodiment, the target gene is a gene that is restrictively expressed in oligodendrocytes. Oligodendrocytes are the terminally differentiated, myelinating cells of the vertebrate central nervous system (CNS) that are responsible for the ensheathment of receptive neuronal axons which is vital for the rapid propagation of nerve impulses. The differentiation of oligodendrocyte progenitor cells (OPCs) into oligodendrocytes and their subsequent myelination of axons are highly regulated processes. Genes that are selectively or restrictively expressed in oligodendrocytes include, without limitation, the transcription regulator SRY-box 10 (SOX10) (Stolt et al., “Terminal Differentiation of Myelin-Forming Oligodendrocytes Depends on the Transcription Factor Sox10,” Genes and Dev. 16:165-170 (2002), which is hereby incorporated by reference in its entirety); the membrane-associated transcription factor, Myelin Regulatory Factor (MYRF) (Bujalka et al., “MYRF is a Membrane-Associated Transcription Factor that Autoproteolytically Cleaves to Directly Activate Myelin Genes,” PLoS Biol. 11(8): e1001625 (2013), which is hereby incorporated by reference in its entirety); Myelin-associated Glycoprotein (MAG); and Myelin Basic Protein (MBP).

In one embodiment, the recombinant genetic construct described herein is designed for insertion into any one of the SOX10, MYRF, MAG, or MBP genes such that the expression of the recombinant construct is coupled to the expression of the gene in oligodendrocytes. In accordance with this embodiment, the first and second gene sequences are derived from SOX10, MYRF, MAG, or MBP genes.

In one embodiment, the recombinant genetic construct is designed to be inserted at or around the 3′ untranslated region of any one of the aforementioned genes, with the first and second gene sequences of the recombinant genetic construct being homologous to regions of the selected gene that are 5′ and 3′, respectively, to the chosen insertion site. The specific location of the insertion site can vary and, thus, the particular sequences of the first and second gene sequences of the recombinant construct will likewise vary. However, selection of these parameters is well within the level of one of skill in the art using the known sequence and structure of each of these genes which is readily available in the art, e.g., via the NCBI gene database and Gene ID No.

In another embodiment, the target gene is a gene that is restrictively expressed in astrocytes. Astrocytes are the most abundant terminally differentiate cell type within the CNS and perform a variety of tasks, from axon guidance and synaptic support, to the control of the blood brain barrier and blood flow.

Terminally differentiated astrocytes may be identified by the presence of various cell surface markers including, e.g., glial fibrillary acidic protein (GFAP) and aquaporin-4 (AQP4). Accordingly, genes expressed selectively in astrocytes in which the recombinant construct can be inserted into include, without limitation, GFAP and AQP4. In accordance with this embodiment, the first and second gene sequences are derived from GFAP and AQP4.

In one embodiment, the recombinant genetic construct described herein is inserted into GFAP or AQP4 such that the expression of the recombinant construct is coupled to the expression of GFAP or AQP4. In one embodiment, the recombinant genetic construct is inserted at or around the 3′ untranslated region of GFAP or AQP4, with the first and second gene sequences of the recombinant genetic construct being homologous to regions of GFAP or AQP4 that are 5′ and 3′, respectively, to the chosen insertion site. The specific location of the insertion site can vary, and thus, the particular sequences of the first and second cell specific gene sequences of the recombinant construct will also vary. However, selection of these parameters is well within the level of one of skill in the art using the known sequence and structure of each of these genes which is readily available in the art.

In another embodiment, the target gene is a gene that is restrictively expressed in neurons. Neurons are electrically excitable cells in the central and peripheral nervous system that function to process and transmit information. Terminally differentiated neurons may be identified by the presence of various cell surface markers including, e.g., synapsin 1 (SYN1), microtubule associated protein 2 (MAP2), and ELAV like RNA binding protein 4 (ELAV4). Accordingly, in one embodiment, the recombinant genetic construct described herein is inserted into any one of the SYN1, MAP2, or ELAV4 such that the expression of the recombinant construct is coupled to the expression of any one of SYN1, MAP2, or ELAV4 gene in neurons. In accordance with this embodiment, the first and second gene sequences are from the SYN1, MAP2, or ELAV4 genes.

In embodiments where it is desirable to restrict expression of the recombinant genetic construct to a particular type of neuron, e.g., a dopaminergic neuron, the recombinant genetic construct is inserted into a gene that is restrictively expressed in the desired neuronal populations. Thus, in one embodiment the recombinant genetic construct described herein is designed for insertion into the tyrosine hydroxylase gene (TH) or the DOPA decarboxylase gene (DDC), which are genes selectively expressed in dopaminergic neurons. In another embodiment, the recombinant genetic construct is designed for insertion into the gene encoding glutamate decarboxylase 2 (GAD2, also known as GAD65) or the gene encoding glutamate decarboxylase 1 (GAD1, also known as GAD67), which are genes selectively expressed in medium spiny neurons and cortical interneurons. In another embodiment the recombinant genetic construct described herein is inserted into the choline O-acetyltransferase gene (CHAT), which is selectively expressed in cholinergic neurons.

In one embodiment, the recombinant genetic construct is inserted at or around the 3′ untranslated region of any one of the neuronal specific genes described above (i.e., SYN1, MAP2, ELAV4, TH, DDC, GAD65, GAD67, or CHAT), with the first and second gene sequences of the recombinant genetic construct being homologous to regions that are 5′ and 3′, respectively, to the chosen insertion site. The specific location of the insertion site may vary and, thus, the specific sequences of the first and second gene sequences of the recombinant construct will also vary. However, the selection of these parameters is well within the level of one of skill in the art using the known sequence and structure of each of these genes which is readily available in the art.

In another embodiment, the target gene is a gene that is expressed in a terminally differentiated cell outside of the central nervous system (CNS). Exemplary terminally differentiated non-CNS cells include, without limitation, adipocytes, chondrocytes, endothelial cells, epithelial cells (keratinocytes, melanocytes), bone cells (osteoblasts, osteoclasts), liver cells (cholangiocytes, hepatocytes), muscle cells (cardiomyocytes, skeletal muscle cells, smooth muscle cells), retinal cells (ganglion cells, muller cells, photoreceptor cells), retinal pigment epithelial cells, renal cells (podocytes, proximal tubule cells, collecting duct cells, distal tubule cells), adrenal cells (cortical adrenal cells, medullary adrenal cells), pancreatic cells (alpha cells, beta cells, delta cells, epsilon cells, pancreatic polypeptide producing cells, exocrine cells); lung cells, bone marrow cells (early B-cell development, early T-cell development, macrophages, monocytes), urothelial cells, fibroblasts, parathyroid cells, thyroid cells, hypothalamic cells, pituitary cells, salivary gland cells, ovarian cells, and testicular cells. Exemplary terminally differentiated non-CNS cells and gene targets selectively expressed in these cells are identified in Table 9 below.

TABLE 9
Exemplary Non-CNS Cells and Gene Targets Selectively Expressed
Therein

| Terminally Differentiated | Cell Specific Gene | | |
|---|---|---|---|
| Cell Type | Target | Organism | Gene ID: |
| Adipocytes | ADIPOQ (ACRP30) | Human | 9370 |
| | | Mouse | 11450 |
| | FABP4 | Human | 2167 |
| | | Mouse | 11770 |
| | PPARG | Human | 5468 |
| | | Mouse | 19016 |
| Chondrocytes | ACAN (AGC1) | Human | 176 |
| | | Mouse | 11595 |
| | COL10A1 | Human | 1300 |
| | | Mouse | 12813 |
| | COMP | Human | 1311 |
| | | Mouse | 12845 |
| Endothelial cells (general) | CDH5 | Human | 1003 |
| | | Mouse | 12562 |
| | KDR (VEGFR3) | Human | 3791 |
| | | Mouse | 16542 |
| | PECAM1 | Human | 5175 |
| | | Mouse | 18613 |
| Endothelial cells (arterial) | DLL4 | Human | 54567 |
| | | Mouse | 54485 |
| | EFNB2 | Human | 1948 |
| | | Mouse | 13642 |
| | NRP1 | Human | 8829 |
| | | Mouse | 18186 |
| Endothelial cells (lymphatic) | LYVE1 | Human | 10894 |
| | | Mouse | 114332 |
| | PROX1 | Human | 5629 |
| | | Mouse | 19130 |
| Endothelial cells (venous) | NR2F2 | Human | 7026 |
| | | Mouse | 11819 |
| Epithelial cells | NRP2 | Human | 8828 |
| (keratinocytes) | | Mouse | 18187 |
| | KRT1 | Human | 3848 |
| | | Mouse | 16678 |
| | KRT10 | Human | 3858 |
| | | Mouse | 16661 |
| | KRT14 | Human | 3861 |
| | | Mouse | 16664 |
| Epithelial cells | PMEL (SILV) | Human | 6490 |
| (melanocytes) | | Mouse | 20431 |
| | TYR | Human | 7299 |
| | | Mouse | 22173 |
| | TYRP1 | Human | 7306 |
| | | Mouse | 22178 |
| Bone Cells (Osteoblasts) | BGLAP | Human | 632 |
| | | Mouse | 12096 |
| | COL2A1 | Human | 1280 |
| | | Mouse | 12824 |
| | IBSP | Human | 3381 |
| | | Mouse | 15891 |
| Bone Cells (Osteoclasts) | CALCR | Human | 799 |
| | | Mouse | 12311 |
| | CTSK | Human | 1513 |
| | | Mouse | 13038 |
| Liver Cells (Cholangiocytes) | ITGB4 | Human | 3691 |
| | | Mouse | 192897 |
| | KRT19 | Human | 3880 |
| | | Mouse | 16669 |
| Liver Cells (Hepatocytes) | ALB | Human | 213 |
| | | Mouse | 11657 |
| | G6PC | Human | 2538 |
| | | Mouse | 14377 |
| | TAT | Human | 6898 |
| | | Mouse | 234724 |
| Muscle Cells | MYH6 | Human | 4624 |
| (cardiomyocytes) | | Mouse | 17888 |
| | MYH7 | Human | 4625 |
| | | Mouse | 140781 |
| | NPPA | Human | 4878 |
| | | Mouse | 230899 |
| Muscle Cells (skeletal | CAV3 | Human | 859 |
| muscle cells) | | Mouse | 12391 |
| | MYH1 | Human | 4619 |
| | | Mouse | 17879 |
| | MYOD1 | Human | 4654 |
| | | Mouse | 17927 |
| Muscle Cells (smooth | MYH11 | Human | 4629 |
| muscle cells) | | Mouse | 17880 |
| | SMTN | Human | 6525 |
| | | Mouse | 29856 |
| | TAGLN | Human | 6876 |
| | | Mouse | 21345 |
| Retinal Cells (ganglion cells) | POU4F2 | Human | 5458 |
| | | Mouse | 18997 |
| Retinal Cells (muller cells) | RLBP1 | Human | 6017 |
| | | Mouse | 19771 |
| Retinal Cells (photoreceptor | PDE6B | Human | 5158 |
| cells) | | Mouse | 18587 |
| | RCVRN | Human | 5957 |
| | | Mouse | 19674 |
| Retinal Pigment Epithelial | PMEL17 | Human | 6490 |
| Cells | | Mouse | 20431 |
| | TYRP1 | Human | 7306 |
| | | Mouse | 22178 |
| | BEST | Human | 7439 |
| | | Mouse | 24115 |
| | CRALBP | Human | 6017 |
| | | Mouse | 19771 |
| | RPE65 | Human | 6121 |
| | | Mouse | 19892 |
| Renal Cells (podocytes) | NPHS2 | Human | 7827 |
| | | Mouse | 170484 |
| Renal Cells (proximal tubule | AQP1 | Human | 358 |
| cells) | | Mouse | 11826 |
| | CYP27B1 | Human | 1594 |
| | | Mouse | 13115 |
| | MIOX | Human | 55586 |
| | | Mouse | 56727 |
| Renal Cells (collecting duct | AQP2 | Human | 359 |
| cells) | | Mouse | 11827 |
| Renal Cells (distal tubule | UMOD | Human | 7369 |
| cells) | | Mouse | 22242 |
| Adrenal Cells (cortical cells) | CYP11A1 | Human | 1583 |
| | | Mouse | 13070 |
| | HSD3B2 | Human | 3284 |
| | | Mouse | 15493 |
| | FDX1 | Human | 2230 |
| | | Mouse | 14148 |
| Adrenal Cells (medullary | PNMT | Human | 5409 |
| cells) | | Mouse | 18948 |
| | DBH | Human | 1621 |
| | | Mouse | 13166 |
| Pancreatic Cells (alpha cells) | GCG | Human | 2641 |
| | | Mouse | 14526 |
| | MAFB | Human | 9935 |
| | | Mouse | 16658 |
| | POU3F4 | Human | 5456 |
| | | Mouse | 18994 |
| Pancreatic Cells (beta cells) | INS | Human | 3630 |
| | | Mouse | 16334 |
| | MAFA | Human | 389692 |
| | | Mouse | 378435 |
| | SLC2A2 | Human | 6514 |
| | | Mouse | 20526 |
| Pancreatic Cells (delta cells) | SST | Human | 6750 |
| | | Mouse | 20604 |
| Pancreatic Cells (epsilon | GHRL (Ghrelin, | Human | 51738 |
| cells) | Obestatin) | Mouse | 58991 |
| Pancreatic Cells (pancreatic | PPY | Human | 5539 |
| polypeptide producing cells) | | Mouse | 19064 |
| Pancreatic Cells (exocrine | CPA1 | Human | 1357 |
| cells) | | Mouse | 109697 |
| Lung Cells | SFTPB | Human | 6439 |
| | | Mouse | 20388 |
| | SFTPC | Human | 6440 |
| | | Mouse | 20389 |
| | SFTPD | Human | 6441 |
| | | Mouse | 20390 |
| Bone Marrow Cells (early B- | CD79A | Human | 973 |
| cell development) | | Mouse | 12518 |
| Bone Marrow Cells (early T- | CD3E | Human | 916 |
| cell development) | | Mouse | 12501 |
| | PTCRA | Human | 171558 |
| | | Mouse | 19208 |
| Bone Marrow Cells | CCR5 | Human | 1234 |
| (macrophages) | | Mouse | 12774 |
| | CXCR4 | Human | 7852 |
| | | Mouse | 12767 |
| | EMR1 | Human | 2015 |
| | | Mouse | 13733 |
| Bone Marrow Cells | ITGAM | Human | 3684 |
| (monocytes) | | Mouse | 16409 |
| Urothelial Cells | UPK2 | Human | 7379 |
| | | Mouse | 22269 |
| Fibroblasts | COL1A2 | Human | 1278 |
| | | Mouse | 12843 |
| | COL3A1 | Human | 1281 |
| | | Mouse | 12825 |
| Parathyroid Cells | PTH | Human | 5741 |
| | | Mouse | 19226 |
| | CASR | Human | 846 |
| | | Mouse | 12374 |
| Thyroid Cells | NIS | Human | 6585 |
| | | Mouse | 114479 |
| | TSHR | Human | 7253 |
| | | Mouse | 22095 |
| | TPO | Human | 7173 |
| | | Mouse | 22018 |
| | TG | Human | 7038 |
| | | Mouse | 21819 |
| Hypothalamic cells | POMC | Human | 5443 |
| | | Mouse | 18976 |
| | MC4R | Human | 4160 |
| | | Mouse | 17202 |
| Pituitary cells | GH1 | Human | 2688 |
| | | Mouse | 14599 |
| | PRL | Human | 5617 |
| | | Mouse | 19109 |
| | TSHB | Human | 7252 |
| | | Mouse | 22094 |
| | FSHB | Human | 2488 |
| | | Mouse | 14308 |
| | LHB | Human | 3972 |
| | | Mouse | 16866 |
| | PRL | Human | 5617 |
| | | Mouse | 19109 |
| Salivary Gland Cells | PRB1 | Human | 5542 |
| | | Mouse | 381833 |
| | PRH1 | Human | 5554 |
| | | Mouse | 19131 |
| | AMY1A | Human | 276 |
| | | Mouse | 11722 |
| | MUC7 | Human | 4589 |
| | | Mouse | 17830 |
| Ovarian Cells | AMHR2 | Human | 269 |
| | | Mouse | 110542 |
| | FSHR | Human | 2492 |
| | | Mouse | 14309 |
| | CYP19A1 | Human | 1588 |
| | | Mouse | 13075 |
| Testicular Cells | PTGDS | Human | 5730 |
| | | Mouse | 19215 |
| | DLK1 | Human | 8788 |
| | | Mouse | 13386 |

In one embodiment, the recombinant genetic construct described herein is designed for insertion into any one of the genes provided in Table 9 such that the expression of the recombinant construct is coupled to the expression of the particular gene in the desired cell. In one embodiment, the recombinant genetic construct is inserted at or around the 3′ untranslated region of any one of the aforementioned genes, with the first and second gene sequences of the recombinant genetic construct being homologous to regions of the selected gene that are 5′ and 3′, respectively, to the chosen insertion site. The specific location of the insertion site can vary and, thus, the particular sequences of the first and second cell specific gene sequences of the recombinant construct will likewise vary. However, selection of these parameters is well within the level of one of skill in the art using the known sequence and structure of each of these genes which is readily available in the art, e.g., via the NCBI gene database and provided Gene ID No.

In some embodiments, the recombinant genetic construct further comprises one or more self-cleaving peptide encoding nucleotide sequences, where the self-cleaving peptide encoding nucleotide sequences are positioned within the construct in a manner effective to mediate the translation of the one or more immune checkpoint proteins in vivo. A “self-cleaving peptide” is a 18-22 amino-acid long viral oligopeptide sequence that mediates ribosome skipping during translation in eukaryotic cells (Liu et al., “Systemic Comparison of 2A peptides for Cloning Multi-Genes in a Polycistronic Vector,” Scientific Reports 7: Article Number 2193 (2017), which is hereby incorporated by reference in its entirety). A non-limiting example of such a self-cleaving peptide is Peptide 2A, which is a short protein sequences first discovered in picornaviruses. Peptide 2A functions by making ribosomes skip the synthesis of a peptide bond at the C-terminus of a 2A element, resulting in a separation between the end of the 2A sequence and the peptide downstream thereof. This “cleavage” occurs between the glycine and proline residues at the C-terminus. Thus, successful ribosome skipping and recommencement of translation results in individual “cleaved” proteins where the protein upstream of the 2A element is attached to the complete 2A peptide except for the C-terminal proline and the protein downstream of the 2A element is attached to one proline at the N-terminus (Liu et al., “Systemic Comparison of 2A peptides for Cloning Multi-Genes in a Polycistronic Vector,” Scientific Reports 7: Article Number 2193 (2017), which is hereby incorporated by reference in its entirety).

Exemplary self-cleaving peptides that can be incorporated in the recombinant genetic construct include, without limitation, porcine teschovirus-1 2A (P2A), Foot and mouth disease virus 2A (F2A), those assign a virus 2A (T2A), equine rhinitis A virus 2A (E2A), cytoplasmic polyhedrosis virus (BmCPV 2A), and flacherie virus (BmIFV 2A). The nucleotide sequences encoding these self-cleaving peptides that are suitable for inclusion in the recombinant genetic construct described herein are provided in Table 10 below.

TABLE 10
Suitable Self-Cleaving Peptide Coding Nucleotide Sequences

| Self-Cleaving Peptide | Nucleotide Sequence* | SEQ ID NO. |
|---|---|---|
| Porcine teschovirus-1 2A | GGAAGCGGAG CTACTAACTT | 24 |
| (P2A) | CAGCCTGCTG AAGCAGGCTG | |
| | GAGACGTGGA GGAGAACCCT GGACCT | |
| Porcine teschovirus-1 2A | GGTTCCGGAG CCACGAACTT | 25 |
| (P2A), codon optimized | CTCTCTGTTA AAGCAAGCAG | |
| | GAGACGTGGA AGAAAACCCC | |
| | GGTCCC | |
| Foot and mouth disease virus | GGAAGCGGAG TGAAACAGAC | 26 |
| 2A (F2A) | TTTGAATTTT GACCTTCTCA | |
| | AGTTGGCGGG AGACGTGGAG | |
| | TCCAACCCTG GACCT | |
| Thosea asigna virus 2A | GAGGGCAGAG GAAGTCTTCT | 27 |
| (T2A) | AACATGCGGT GACGTGGAGG | |
| | AGAATCCCGG CCCT | |
| Equine rhinitis A virus 2A | GGAAGCGGAC AGTGTACTAA | 28 |
| (E2A) | TTATGCTCTC TTGAAATTGG | |
| | CTGGAGATGT TGAGAGCAAC | |
| | CCTGGACCT | |
| Cytoplasmic polyhedrosis | GACGTTTTTC GCTCTAATTA | 29 |
| virus (BmCPV 2A) | TGACCTACTA AAGTTGTGCG | |
| | GTGATATCGA GTCTAATCCT GGACCT | |
| Flacherie virus (BmIFV 2A) | ACTCTGACGA GGGCGAAGAT | 30 |
| | TGAGGATGAA TTGATTCGTG | |
| | CAGGAATTGA ATCAAATCCT GGACCT | |

*See Wang et al., “2A Self-Cleaving Peptide-Based Multi-Gene Expression System in the Silkworm Bombyx mori,” Sci. Rep. 5:16273 (2015) and U.S. Pat. Application Publication No. 2018/0369280 to Schmitt et al., which are hereby incorporated by reference in their entirety.

In some embodiments, the recombinant genetic construct further comprises an inducible cell death gene positioned within the construct in a manner effective to achieve inducible cell suicide. An inducible cell death gene refers to a genetically encoded element that allows selective destruction of expressing cells in the face of unacceptable toxicity by administration of an activating pharmaceutical agent.

Several inducible cell death genes are well known in the art and suitable for inclusion in the recombinant genetic construct described herein (see Stavrou et al., “A Rapamycin-Activated Caspase 9-Based Suicide Gene,” Mol. Ther. 26(5):1266-1276 (2018), which is hereby incorporated by reference in its entirety). Exemplary suicide genes include, without limitation, RQR8 and huEGFRt, which are surface proteins recognized by therapeutic monoclonal antibodies (mAbs); herpes simplex virus thymidine kinase (HSV-TK), an inducible cell death gene activated by the small molecule ganciclovir; inducible caspase 9 (iCasp9), a fusion of mutated FKBP12 with the catalytic domain of caspase 9 which allows docking of a small molecular chemical inducer of dimerization (CID, AP1903/AP20187); rapamycin-activated caspase 9 (rapaCasp9), an inducible cell death gene activated by rapamycin (Stavrou et al., “A Rapamycin-Activated Caspase 9-Based Suicide Gene,” Mol. Ther. 26(5):1266-1276 (2018), which is hereby incorporated by reference in its entirety); and inducible caspase-3 (iCasp3), a fusion of mutated FK506 binding domains with caspase-3 which allows docking of a CID (AP20187) (Ono et al., “Exposure to Sequestered Self-Antigens in vivo is not Sufficient for the Induction of Autoimmune Diabetes,” PLos One 12(3):e0173176 (2017) and MacCorkle et al., “Synthetic Activation of Caspases: Artificial Death Switches,” PNAS 95(7): 3655-3660 (1998), which are hereby incorporated by reference in their entirety). In another embodiment, the recombinant genetic construct contains an inducible cell death gene linked to the expression of a cell-division gene, like the cell-division gene (CDK1) (Liang et al., “Linking a Cell-Division Gene and a Suicide Gene to Define and Improve Cell Therapy Safety,” Nature 563:701-704 (2018), which is hereby incorporated by reference in its entirety).

In some embodiments, the recombinant genetic construct further comprises a selection marker. Suitable selection markers for mammalian cells are known in the art, and include for example, thymidine kinase, dihydrofolate reductase (together with methotrexate as a DHFR amplifier), aminoglycoside phosphotransferase, hygromycin B phosphotransferase, asparagine synthetase, adenosine deaminase, metallothionein, and antibiotic resistant genes, e.g., the puromycin resistance gene or the neomycin resistance gene. Exemplary antibiotic resistance gene sequences that can be used as selection markers in the recombinant genetic construct as described herein are provided in Table 11 below.

TABLE 11
Suitable Selection Marker Gene Sequences

| Promoter | | SEQ ID |
|---|---|---|
| Name | Nucleotide Sequence* | NO. |
| Puromycin | ATGACCGAGTACAAGCCCACGGTGCGCCTCGCCACCCGCGACGA | 31 |
| Resistance | CGTCCCCAGGGCCGTACGCACCCTCGCCGCCGCGTTCGCCGACT | |
| | ACCCCGCCACGCGCCACACCGTCGATCCGGACCGCCACATCGAG | |
| | CGGGTCACCGAGCTGCAAGAACTCTTCCTCACGCGCGTCGGGCT | |
| | CGACATCGGCAAGGTGTGGGTCGCGGACGACGGCGCCGCGGTGG | |
| | CGGTCTGGACCACGCCGGAGGGCGTCGAAGCGGGGGCGGTGTTC | |
| | GCCGAGATCGGCCCGCGCATGGCCGAGTTGAGCGGTTCCCGGCT | |
| | GGCCGCGCAGCAACAGATGGAAGGCCTCCTGGCGCCGCACCGGC | |
| | CCAAGGAGCCCGCGTGGTTCCTGGCCACCGTCGGCGTCTCGCCC | |
| | GACCACCAGGGCAAGGGTCTGGGCAGCGCCGTCGTGCTCCCCGG | |
| | AGTGGAGGCGGCCGAGCGCGCCGGGGTGCCCGCCTTCCTGGAGA | |
| | CCTCCGCGCCCCGCAACCTCCCCTTCTACGAGCGGCTCGGCTTC | |
| | ACCGTCACCGCCGACGTCGAGGTGCCCGAAGGACCGCGCACCTG | |
| | GTGCATGACCCGCAAGCCCGGTGCCTGA | |
| Neomycin | ATGAGCCATATTCAACGGGAAACGTCTTGCTCTAGGCCGCGATT | 32 |
| Resistance | AAATTCCAACATGGATGCTGATTTATATGGGTATAAATGGGCTC | |
| | GCGATAATGTCGGGCAATCAGGTGCGACAATCTATCGATTGTAT | |
| | GGGAAGCCCGATGCGCCAGAGTTGTTTCTGAAACATGGCAAAGG | |
| | TAGCGTTGCCAATGATGTTACAGATGAGATGGTCAGACTAAACT | |
| | GGCTGACGGAATTTATGCCTCTTCCGACCATCAAGCATTTTATC | |
| | CGTACTCCTGATGATGCATGGTTACTCACCACTGCGATCCCCGG | |
| | GAAAACAGCATTCCAGGTATTAGAAGAATATCCTGATTCAGGTG | |
| | AAAATATTGTTGATGCGCTGGCAGTGTTCCTGCGCCGGTTGCAT | |
| | TCGATTCCTGTTTGTAATTGTCCTTTTAACAGCGATCGCGTATT | |
| | TCGTCTCGCTCAGGCGCAATCACGAATGAATAACGGTTTGGTTG | |
| | ATGCGAGTGATTTTGATGACGAGCGTAATGGCTGGCCTGTTGAA | |
| | CAAGTCTGGAAAGAAATGCATAAACTTTTGCCATTCTCACCGGA | |
| | TTCAGTCGTCACTCATGGTGATTTCTCACTTGATAACCTTATTT | |
| | TTGACGAGGGGAAATTAATAGGTTGTATTGATGTTGGACGAGTC | |
| | GGAATCGCAGACCGATACCAGGATCTTGCCATCCTATGGAACTG | |
| | CCTCGGTGAGTTTTCTCCTTCATTACAGAAACGGCTTTTTCAAA | |
| | AATATGGTATTGATAATCCTGATATGAATAAATTGCAGTTTCAT | |
| | TTGATGCTCGATGAGTTTTTCTAA | |
| Hygromycin | ATGAAAAAGCCTGAACTCACCGCGACGTCTGTCGAGAAGTTTCT | 33 |
| B | GATCGAAAAGTTCGACAGCGTCTCCGACCTGATGCAGCTCTCGG | |
| | AGGGCGAAGAATCTCGTGCTTTCAGCTTCGATGTAGGAGGGCGT | |
| | GGATATGTCCTGCGGGTAAATAGCTGCGCCGATGGTTTCTACAA | |
| | AGATCGTTATGTTTATCGGCACTTTGCATCGGCCGCGCTCCCGA | |
| | TTCCGGAAGTGCTTGACATTGGGGAGTTCAGCGAGAGCCTGACC | |
| | TATTGCATCTCCCGCCGTGCACAGGGTGTCACGTTGCAAGACCT | |
| | GCCTGAAACCGAACTGCCCGCTGTTCTCGAGCCGGTCGCGGAGG | |
| | CGATGGATGCGATCGCTGCGGCCGATCTTAGCCAGACGAGCGGG | |
| | TTCGGCCCATTCGGACCGCAAGGAATCGGTCAATACACTACATG | |
| | GCGTGATTTCATATGCGCGATTGCTGATCCCCATGTGTATCACT | |
| | GGCAAACTGTGATGGACGACACCGTCAGTGCGTCCGTCGCGCAG | |
| | GCTCTCGATGAGCTGATGCTTTGGGCCGAGGACTGCCCCGAAGT | |
| | CCGGCACCTCGTGCATGCGGATTTCGGCTCCAACAATGTCCTGA | |
| | CGGACAATGGCCGCATAACAGCGGTCATTGACTGGAGCGAGGCG | |
| | ATGTTCGGGGATTCCCAATACGAGGTCGCCAACATCCTCTTCTG | |
| | GAGGCCGTGGTTGGCTTGTATGGAGCAGCAGACGCGCTACTTCG | |
| | AGCGGAGGCATCCGGAGCTTGCAGGATCGCCGCGCCTCCGGGCG | |
| | TATATGCTCCGCATTGGTCTTGACCAACTCTATCAGAGCTTGGT | |
| | TGACGGCAATTTCGATGATGCAGCTTGGGCGCAGGGTCGATGCG | |
| | ACGCAATCGTCCGATCCGGAGCCGGGACTGTCGGGCGTACACAA | |
| | ATCGCCCGCAGAAGCGCGGCCGTCTGGACCGATGGCTGTGTAGA | |
| | AGTACTCGCCGATAGTGGAAACCGACGCCCCAGCACTCGTCCGA | |
| | GGGCAAAGGAATAG | |

When the recombinant genetic construct comprises a mammalian selection marker, the selection marker may be operatively linked to a constitutive mammalian promoter.

Exemplary constitutive mammalian promoters suitable for inclusion in the recombinant construct described herein are well known in the art and are shown in Table 12 below (Qin et al., “Systematic Comparison of Constitutive Promoters and the Doxycycline-Inducible Promoter,” PLoS One 5(5):e10611 (2010), which is hereby incorporated by reference in its entirety).

TABLE 12
Suitable Promoter Sequences

| Promoter | | SEQ ID |
|---|---|---|
| Name | Nucleotide Sequence* | NO. |
| UBC | GGTGCAGCGGCCTCCGCGCCGGGTTTTGGCGCCTCCCGCGGGCGC | 34 |
| | CCCCCTCCTCACGGCGAGCGCTGCCACGTCAGACGAAGGGCGCAG | |
| | GAGCGTTCCTGATCCTTCCGCCCGGACGCTCAGGACAGCGGCCCG | |
| | CTGCTCATAAGACTCGGCCTTAGAACCCCAGTATCAGCAGAAGGA | |
| | CATTTTAGGACGGGACTTGGGTGACTCTAGGGCACTGGTTTTCTT | |
| | TCCAGAGAGCGGAACAGGCGAGGAAAAGTAGTCCCTTCTCGGCGA | |
| | TTCTGCGGAGGGATCTCCGTGGGGCGGTGAACGCCGATGATTATA | |
| | TAAGGACGCGCCGGGTGTGGCACAGCTAGTTCCGTCGCAGCCGGG | |
| | ATTTGGGTCGCGGTTCTTGTTTGTGGATCGCTGTGATCGTCACTT | |
| | GGTGAGTTGCGGGCTGCTGGGCTGGCCGGGGCTTTCGTGGCCGCC | |
| | GGGCCGCTCGGTGGGACGGAAGCGTGTGGAGAGACCGCCAAGGGC | |
| | TGTAGTCTGGGTCCGCGAGCAAGGTTGCCCTGAACTGGGGGTTGG | |
| | GGGGAGCGCACAAAATGGCGGCTGTTCCCGAGTCTTGAATGGAAG | |
| | ACGCTTGTAAGGCGGGCTGTGAGGTCGTTGAAACAAGGTGGGGGG | |
| | CATGGTGGGCGGCAAGAACCCAAGGTCTTGAGGCCTTCGCTAATG | |
| | CGGGAAAGCTCTTATTCGGGTGAGATGGGCTGGGGCACCATCTGG | |
| | GGACCCTGACGTGAAGTTTGTCACTGACTGGAGAACTCGGGTTTG | |
| | TCGTCTGGTTGCGGGGGCGGCAGTTATGCGGTGCCGTTGGGCAGT | |
| | GCACCCGTACCTTTGGGAGCGCGCGCCTCGTCGTGTCGTGACGTC | |
| | ACCCGTTCTGTTGGCTTATAATGCAGGGTGGGGCCACCTGCCGGT | |
| | AGGTGTGCGGTAGGCTTTTCTCCGTCGCAGGACGCAGGGTTCGGG | |
| | CCTAGGGTAGGCTCTCCTGAATCGACAGGCGCCGGACCTCTGGTG | |
| | AGGGGAGGGATAAGTGAGGCGTCAGTTTCTTTGGTCGGTTTTATG | |
| | TACCTATCTTCTTAAGTAGCTGAAGCTCCGGTTTTGAACTATGCG | |
| | CTCGGGGTTGGCGAGTGTGTTTTGTGAAGTTTTTTAGGCACCTTT | |
| | TGAAATGTAATCATTTGGGTCAATATGTAATTTTCAGTGTTAGAC | |
| | TAGTAAA | |
| PGK | TTCTACCGGGTAGGGGAGGCGCTTTTCCCAAGGCAGTCTGGAGCA | 35 |
| | TGCGCTTTAGCAGCCCCGCTGGGCACTTGGCGCTACACAAGTGGC | |
| | CTCTGGCCTCGCACACATTCCACATCCACCGGTAGGCGCCAACCG | |
| | GCTCCGTTCTTTGGTGGCCCCTTCGCGCCACCTTCTACTCCTCCC | |
| | CTAGTCAGGAAGTTCCCCCCCGCCCCGCAGCTCGCGTCGTGCAGG | |
| | ACGTGACAAATGGAAGTAGCACGTCTCACTAGTCTCGTGCAGATG | |
| | GACAGCACCGCTGAGCAATGGAAGCGGGTAGGCCTTTGGGGCAGC | |
| | GGCCAATAGCAGCTTTGCTCCTTCGCTTTCTGGGCTCAGAGGCTG | |
| | GGAAGGGGTGGGTCCGGGGGCGGGCTCAGGGGCGGGCTCAGGGGC | |
| | GGGGCGGGCGCCCGAAGGTCCTCCGGAGGCCCGGCATTCTGCACG | |
| | CTTCAAAAGCGCACGTCTGCCGCGCTGTTCTCCTCTTCCTCATCT | |
| | CCGGGCCTTTCGACCT | |
| EF1a | GGCTCCGGTGCCCGTCAGTGGGCAGAGCGCACATCGCCCACAGTC | 36 |
| | CCCGAGAAGTTGGGGGGAGGGGTCGGCAATTGAACCGGTGCCTAG | |
| | AGAAGGTGGCGCGGGGTAAACTGGGAAAGTGATGTCGTGTACTGG | |
| | CTCCGCCTTTTTCCCGAGGGTGGGGGAGAACCGTATATAAGTGCA | |
| | GTAGTCGCCGTGAACGTTCTTTTTCGCAACGGGTTTGCCGCCAGA | |
| | ACACAGGTAAGTGCCGTGTGTGGTTCCCGCGGGCCTGGCCTCTTT | |
| | ACGGGTTATGGCCCTTGCGTGCCTTGAATTACTTCCACCTGGCTG | |
| | CAGTACGTGATTCTTGATCCCGAGCTTCGGGTTGGAAGTGGGTGG | |
| | GAGAGTTCGAGGCCTTGCGCTTAAGGAGCCCCTTCGCCTCGTGCT | |
| | TGAGTTGAGGCCTGGCCTGGGCGCTGGGGCCGCCGCGTGCGAATC | |
| | TGGTGGCACCTTCGCGCCTGTCTCGCTGCTTTCGATAAGTCTCTA | |
| | GCCATTTAAAATTTTTGATGACCTGCTGCGACGCTTTTTTTCTGG | |
| | CAAGATAGTCTTGTAAATGCGGGCCAAGATCTGCACACTGGTATT | |
| | TCGGTTTTTGGGGCCGCGGGCGGCGACGGGGCCCGTGCGTCCCAG | |
| | CGCACATGTTCGGCGAGGCGGGGCCTGCGAGCGCGGCCACCGAGA | |
| | ATCGGACGGGGGTAGTCTCAAGCTGGCCGGCCTGCTCTGGTGCCT | |
| | GGTCTCGCGCCGCCGTGTATCGCCCCGCCCTGGGCGGCAAGGCTG | |
| | GCCCGGTCGGCACCAGTTGCGTGAGCGGAAAGATGGCCGCTTCCC | |
| | GGCCCTGCTGCAGGGAGCTCAAAATGGAGGACGCGGCGCTCGGGA | |
| | GAGCGGGCGGGTGAGTCACCCACACAAAGGAAAAGGGCCTTTCCG | |
| | TCCTCAGCCGTCGCTTCATGTGACTCCACGGAGTACCGGGCGCCG | |
| | TCCAGGCACCTCGATTAGTTCTCGAGCTTTTGGAGTACGTCGTCT | |
| | TTAGGTTGGGGGGAGGGGTTTTATGCGATGGAGTTTCCCCACACT | |
| | GAGTGGGTGGAGACTGAAGTTAGGCCAGCTTGGCACTTGATGTAA | |
| | TTCTCCTTGGAATTTGCCCTTTTTGAGTTTGGATCTTGGTTCATT | |
| | CTCAAGCCTCAGACAGTGGTTCAAAGTTTTTTTCTTCCATTTCAG | |
| | GTGTCGTGA | |
| CMV | TAGTTATTAATAGTAATCAATTACGGGGTCATTAGTTCATAGCCC | 37 |
| | ATATATGGAGTTCCGCGTTACATAACTTACGGTAAATGGCCCGCC | |
| | TGGCTGACCGCCCAACGACCCCCGCCCATTGACGTCAATAATGAC | |
| | GTATGTTCCCATAGTAACGCCAATAGGGACTTTCCATTGACGTCA | |
| | ATGGGTGGAGTATTTACGGTAAACTGCCCACTTGGCAGTACATCA | |
| | AGTGTATCATATGCCAAGTACGCCCCCTATTGACGTCAATGACGG | |
| | TAAATGGCCCGCCTGGCATTATGCCCAGTACATGACCTTATGGGA | |
| | CTTTCCTACTTGGCAGTACATCTACGTATTAGTCATCGCTATTAC | |
| | CATGGTGATGCGGTTTTGGCAGTACATCAATGGGCGTGGATAGCG | |
| | GTTTGACTCACGGGGATTTCCAAGTCTCCACCCCATTGACGTCAA | |
| | TGGGAGTTTGTTTTGGCACCAAAATCAACGGGACTTTCCAAAATG | |
| | TCGTAACAACTCCGCCCCATTGACGCAAATGGGCGGTAGGCGTGT | |
| | ACGGTGGGAGGTCTATATAAGCAGAGCTGGTTTAGTGAACCGTCA | |
| | GATC | |
| CAGG | ACTAGTTATTAATAGTAATCAATTACGGGGTCATTAGTTCATAGC | 38 |
| | CCATATATGGAGTTCCGCGTTACATAACTTACGGTAAATGGCCCG | |
| | CCTGGCTGACCGCCCAACGACCCCCGCCCATTGACGTCAATAATG | |
| | ACGTATGTTCCCATAGTAACGCCAATAGGGACTTTCCATTGACGT | |
| | CAATGGGTGGAGTATTTACGGTAAACTGCCCACTTGGCAGTACAT | |
| | CAAGTGTATCATATGCCAAGTACGCCCCCTATTGACGTCAATGAC | |
| | GGTAAATGGCCCGCCTGGCATTATGCCCAGTACATGACCTTATGG | |
| | GACTTTCCTACTTGGCAGTACATCTACGTATTAGTCATCGCTATT | |
| | ACCATGGTCGAGGTGAGCCCCACGTTCTGCTTCACTCTCCCCATC | |
| | TCCCCCCCCTCCCCACCCCCAATTTTGTATTTATTTATTTTTTAA | |
| | TTATTTTGTGCAGCGATGGGGGCGGGGGGGGGGGGGGGGCGCGCG | |
| | CCAGGCGGGGCGGGGCGGGGCGAGGGGCGGGGCGGGGCGAGGCGG | |
| | AGAGGTGCGGCGGCAGCCAATCAGAGCGGCGCGCTCCGAAAGTTT | |
| | CCTTTTATGGCGAGGCGGCGGCGGCGGCGGCCCTATAAAAAGCGA | |
| | AGCGCGCGGCGGGCGGGGAGTCGCTGCGACGCTGCCTTCGCCCCG | |
| | TGCCCCGCTCCGCCGCCGCCTCGCGCCGCCCGCCCCGGCTCTGAC | |
| | TGACCGCGTTACTCCCACAGGTGAGCGGGCGGGACGGCCCTTCTC | |
| | CTCCGGGCTGTAATTAGCGCTTGGTTTAATGACGGCTTGTTTCTT | |
| | TTCTGTGGCTGCGTGAAAGCCTTGAGGGGCTCCGGGAGGGCCCTT | |
| | TGTGCGGGGGGAGCGGCTCGGGGGGTGCGTGCGTGTGTGTGTGCG | |
| | TGGGGAGCGCCGCGTGCGGCTCCGCGCTGCCCGGCGGCTGTGAGC | |
| | GCTGCGGGCGCGGCGCGGGGCTTTGTGCGCTCCGCAGTGTGCGCG | |
| | AGGGGAGCGCGGCCGGGGGCGGTGCCCCGCGGTGCGGGGGGGGCT | |
| | GCGAGGGGAACAAAGGCTGCGTGCGGGGTGTGTGCGTGGGGGGGT | |
| | GAGCAGGGGGTGTGGGCGCGTCGGTCGGGCTGCAACCCCCCCTGC | |
| | ACCCCCCTCCCCGAGTTGCTGAGCACGGCCCGGCTTCGGGTGCGG | |
| | GGCTCCGTACGGGGCGTGGCGCGGGGCTCGCCGTGCCGGGCGGGG | |
| | GGTGGCGGCAGGTGGGGGTGCCGGGCGGGGCGGGGCCGCCTCGGG | |
| | CCGGGGAGGGCTCGGGGGAGGGGCGCGGCGGCCCCCGGAGCGCCG | |
| | GCGGCTGTCGAGGCGCGGCGAGCCGCAGCCATTGCCTTTTATGGT | |
| | AATCGTGCGAGAGGGCGCAGGGACTTCCTTTGTCCCAAATCTGTG | |
| | CGGAGCCGAAATCTGGGAGGCGCCGCCGCACCCCCTCTAGCGGGC | |
| | GCGGGGCGAAGCGGTGCGGCGCCGGCAGGAAGGAAATGGGCGGGG | |
| | AGGGCCTTCGTGCGTCGCCGCGCCGCCGTCCCCTTCTCCCTCTCC | |
| | AGCCTCGGGGCTGTCCGCGGGGGGACGGCTGCCTTCGGGGGGGAC | |
| | GGGGCAGGGCGGGGTTCGGCTTCTGGCGTGTGACCGGCGGCTCTA | |
| | GAGCCTCTGCTAACCATGTTCATGCCTTCTTCTTTTTCCTACAGC | |
| | TCCTGGGCAACGTGCTGGTTATTGTGCTGTCTCATCATTTTGGCA | |
| | AAGAATTC | |
| SV40 | CTGTGGAATGTGTGTCAGTTAGGGTGTGGAAAGTCCCCAGGCTCC | 39 |
| | CCAGCAGGCAGAAGTATGCAAAGCATGCATCTCAATTAGTCAGCA | |
| | ACCAGGTGTGGAAAGTCCCCAGGCTCCCCAGCAGGCAGAAGTATG | |
| | CAAAGCATGCATCTCAATTAGTCAGCAACCATAGTCCCGCCCCTA | |
| | ACTCCGCCCATCCCGCCCCTAACTCCGCCCAGTTCCGCCCATTCT | |
| | CCGCCCCATGGCTGACTAATTTTTTTTATTTATGCAGAGGCCGAG | |
| | GCCGCCTCTGCCTCTGAGCTATTCCAGAAGTAGTGAGGAGGCTTT | |
| | TTTGGAGGCCTAGGCTTTTGCAAAAAGCT | |

*See Qin et al., “Systematic Comparison of Constitutive Promoters and the Doxycycline-Inducible Promoter,” PLoS One 5(5):e10611 (2010), which is hereby incorporated by reference in its entirety.

In some embodiments, the recombinant genetic construct further encodes at least one marker domain. Non-limiting examples of marker domains include fluorescent proteins, purification tags, and epitope tags.

In some aspects, the marker domain may be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, mRasberry, mStrawberry, Jred), and orange fluorescent proteins (mOrange, mKO, Kusabira-Orange, Monomeric Kusabira-Orange, mTangerine, tdTomato) or any other suitable fluorescent protein.

In other aspects, the marker domain may be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin.

The marker domain may be operatively coupled to the constitutive mammalian promoter. For example, in some embodiments, the constitutive mammalian promoter is EF1a and the marker domain is operatively coupled to EF1a. In accordance with this embodiment, the marker domain may be CopGFP. Exemplary nucleotide sequences encoding suitable marker domain sequences are shown in Table 13 below.

TABLE 13
Suitable Marker Domain Sequences

| Marker | | |
|---|---|---|
| Domain | | SEQ |
| Name | Nucleotide Sequence | ID NO. |
| CopGFP | AGAGCGACGAGAGCGGCCTGCCCGCCATGGAGATCGAGTGCCGCATC | 40 |
| | ACCGGCACCCTGAACGGCGTGGAGTTCGAGCTGGTGGGCGGCGGAGA | |
| | GGGCACCCCCAAGCAGGGCCGCATGACCAACAAGATGAAGAGCACCA | |
| | AAGGCGCCCTGACCTTCAGCCCCTACCTGCTGAGCCACGTGATGGGC | |
| | TACGGCTTCTACCACTTCGGCACCTACCCCAGCGGCTACGAGAACCC | |
| | CTTCCTGCACGCCATCAACAACGGCGGCTACACCAACACCCGCATCG | |
| | AGAAGTACGAGGACGGCGGCGTGCTGCACGTGAGCTTCAGCTACCGC | |
| | TACGAGGCCGGCCGCGTGATCGGCGACTTCAAGGTGGTGGGCACCGG | |
| | CTTCCCCGAGGACAGCGTGATCTTCACCGACAAGATCATCCGCAGCA | |
| | ACGCCACCGTGGAGCACCTGCACCCCATGGGCGATAACGTGCTGGTG | |
| | GGCAGCTTCGCCCGCACCTTCAGCCTGCGCGACGGCGGCTACTACAG | |
| | CTTCGTGGTGGACAGCCACATGCACTTCAAGAGCGCCATCCACCCCA | |
| | GCATCCTGCAGAACGGGGGCCCCATGTTCGCCTTCCGCCGCGTGGAG | |
| | GAGCTGCACAGCAACACCGAGCTGGGCATCGTGGAGTACCAGCACGC | |
| | CTTCAAGACCCCCATCGCCTTCGCCAGATCCCGCGCTCAGTCGTCCA | |
| | ATTCTGCCGTGGACGGCACCGCCGGACCCGGCTCCACCGGATCTCGC | |
| eGFP | ATGGTGAGCAAGGGCGAGGAGCTGTTCACCGGGGTGGTGCCCATCCT | 41 |
| | GGTCGAGCTGGACGGCGACGTAAACGGCCACAAGTTCAGCGTGTCCG | |
| | GCGAGGGCGAGGGCGATGCCACCTACGGCAAGCTGACCCTGAAGTTC | |
| | ATCTGCACCACCGGCAAGCTGCCCGTGCCCTGGCCCACCCTCGTGAC | |
| | CACCCTGACCTACGGCGTGCAGTGCTTCAGCCGCTACCCCGACCACA | |
| | TGAAGCAGCACGACTTCTTCAAGTCCGCCATGCCCGAAGGCTACGTC | |
| | CAGGAGCGCACCATCTTCTTCAAGGACGACGGCAACTACAAGACCCG | |
| | CGCCGAGGTGAAGTTCGAGGGCGACACCCTGGTGAACCGCATCGAGC | |
| | TGAAGGGCATCGACTTCAAGGAGGACGGCAACATCCTGGGGCACAAG | |
| | CTGGAGTACAACTACAACAGCCACAACGTCTATATCATGGCCGACAA | |
| | GCAGAAGAACGGCATCAAGGTGAACTTCAAGATCCGCCACAACATCG | |
| | AGGACGGCAGCGTGCAGCTCGCCGACCACTACCAGCAGAACACCCCC | |
| | ATCGGCGACGGCCCCGTGCTGCTGCCCGACAACCACTACCTGAGCAC | |
| | CCAGTCCGCCCTGAGCAAAGACCCCAACGAGAAGCGCGATCACATGG | |
| | TCCTGCTGGAGTTCGTGACCGCCGCCGGGATCACTCTCGGCATGGAC | |
| | GAGCTGTACAAG | |
| YFP | ATGGTGAGCAAGGGCGAGGAGCTGTTCACCGGGGTGGTGCCCATCCT | 42 |
| | GGTCGAGCTGGACGGCGACGTAAACGGCCACAAGTTCAGCGTGTCCG | |
| | GCGAGGGCGAGGGCGATGCCACCTACGGCAAGCTGACCCTGAAGTTC | |
| | ATCTGCACCACCGGCAAGCTGCCCGTGCCCTGGCCCACCCTCGTGAC | |
| | CACCTTCGGCTACGGCCTGCAGTGCTTCGCCCGCTACCCCGACCACA | |
| | TGAAGCAGCACGACTTCTTCAAGTCCGCCATGCCCGAAGGCTACGTC | |
| | CAGGAGCGCACCATCTTCTTCAAGGACGACGGCAACTACAAGACCCG | |
| | CGCCGAGGTGAAGTTCGAGGGCGACACCCTGGTGAACCGCATCGAGC | |
| | TGAAGGGCATCGACTTCAAGGAGGACGGCAACATCCTGGGGCACAAG | |
| | CTGGAGTACAACTACAACAGCCACAACGTCTATATCATGGCCGACAA | |
| | GCAGAAGAACGGCATCAAGGTGAACTTCAAGATCCGCCACAACATCG | |
| | AGGACGGCAGCGTGCAGCTCGCCGACCACTACCAGCAGAACACCCCC | |
| | ATCGGCGACGGCCCCGTGCTGCTGCCCGACAACCACTACCTGAGCTA | |
| | CCAGTCCGCCCTGAGCAAAGACCCCAACGAGAAGCGCGATCACATGG | |
| | TCCTGCTGGAGTTCGTGACCGCCGCCGGGATCACTCTCGGCATGGAC | |
| | GAGCTGTACAAGTAA | |
| mCherry | ATGGTGAGCAAGGGCGAGGAGGATAACATGGCCATCATCAAGGAGTT | 43 |
| | CATGCGCTTCAAGGTGCACATGGAGGGCTCCGTGAACGGCCACGAGT | |
| | TCGAGATCGAGGGCGAGGGCGAGGGCCGCCCCTACGAGGGCACCCAG | |
| | ACCGCCAAGCTGAAGGTGACCAAGGGTGGCCCCCTGCCCTTCGCCTG | |
| | GGACATCCTGTCCCCTCAGTTCATGTACGGCTCCAAGGCCTACGTGA | |
| | AGCACCCCGCCGACATCCCCGACTACTTGAAGCTGTCCTTCCCCGAG | |
| | GGCTTCAAGTGGGAGCGCGTGATGAACTTCGAGGACGGCGGCGTGGT | |
| | GACCGTGACCCAGGACTCCTCCCTGCAGGACGGCGAGTTCATCTACA | |
| | AGGTGAAGCTGCGCGGCACCAACTTCCCCTCCGACGGCCCCGTAATG | |
| | CAGAAGAAGACCATGGGCTGGGAGGCCTCCTCCGAGCGGATGTACCC | |
| | CGAGGACGGCGCCCTGAAGGGCGAGATCAAGCAGAGGCTGAAGCTGA | |
| | AGGACGGCGGCCACTACGACGCTGAGGTCAAGACCACCTACAAGGCC | |
| | AAGAAGCCCGTGCAGCTGCCCGGCGCCTACAACGTCAACATCAAGTT | |
| | GGACATCACCTCCCACAACGAGGACTACACCATCGTGGAACAGTACG | |
| | AACGCGCCGAGGGCCGCCACTCCACCGGCGGCATGGACGAGCTGTAC | |
| | AAGTAA | |

In some embodiments, the recombinant genetic construct of the present disclosure is incorporated into a delivery vector. Suitable delivery vectors include, without limitation, plasmid vectors, viral vectors, including without limitation, vaccina vectors, lentiviral vector (integration competent or integration-defective lentiviral vectors), adenoviral vectors, adeno-associated viral vectors, vectors for baculovirus expression, transposon based vectors or any other vector suitable for introduction of the recombinant genetic construct described herein into a cell by any means to facilitate the gene/cell selective expression of the recombinant construct.

Another aspect of the disclosure relates to a preparation of one or more cells comprising the recombinant genetic construct described herein. The preparation may be a preparation of cells from any organism. In some embodiments, the preparation is a preparation of mammalian cells, e.g., a preparation of rodent cells (i.e., mouse or rat cells), rabbit cells, guinea pig cells, feline cells, canine cells, porcine cells, equine cells, bovine cell, ovine cells, monkey cells, or human cells. In one embodiment, the preparation is a preparation of human cells. Suitable cells comprising the recombinant genetic construct as described herein include primary or immortalized embryonic cells, fetal cells, or adult cells, at any stage of their lineage, e.g., totipotent, pluripotent, multipotent, or differentiated cells.

In some embodiments, the preparation is a preparation of pluripotent stem cells. Pluripotent stem cells can give rise to any cell of the three germ layers (i.e., endoderm, mesoderm and ectoderm). In one embodiment, the preparation of cells comprising the recombinant genetic construct is a preparation of induced pluripotent stem cells (iPSCs). In another embodiment, the preparation of cells comprising the recombinant genetic construct is a preparation of pluripotent embryonic stem cells.

In another embodiment, the preparation of one or more cells may be a preparation of multipotent stem cells. Multipotent stem cells can develop into a limited number of cells in a particular lineage. Examples of multipotent stem cells include progenitor cells, e.g., neural progenitor cells which give rise to cells of the central nervous system such as neurons, astrocytes and oligodendrocytes. Progenitor cells are an immature or undifferentiated cell population having the potential to mature and differentiate into a more specialized, differentiated cell type. A progenitor cell can also proliferate to make more progenitor cells that are similarly immature or undifferentiated. Suitable preparations of progenitor cells comprising the recombinant genetic construct include, without limitation, preparations of neural progenitor cells, neuronal progenitor cells, glial progenitor cells, oligodendrocyte-biased progenitor cells, and astrocyte-biased progenitor cells. Other suitable progenitor cell populations include, without limitation, bone marrow progenitor cells, cardiac progenitor cells, endothelial progenitor cells, epithelial progenitor cells, hematopoietic progenitor cells, hepatic progenitor cells, osteoprogenitor cells, muscle progenitor cells, pancreatic progenitor cells, pulmonary progenitor cells, renal progenitor cells, vascular progenitor cells, retinal progenitor cells.

The preparation of cells comprising the recombinant genetic construct as described herein can also be a preparation of terminally differentiated cells. In one embodiment, the preparation of one or more cells may be a preparation of terminally differentiated neurons, oligodendrocytes, or astrocytes. In another embodiment, the preparation of one or more cells comprising the recombinant genetic construct is a preparation of adipocytes, chondrocytes, endothelial cells, epithelial cells (keratinocytes, melanocytes), bone cells (osteoblasts, osteoclasts), liver cells (cholangiocytes, hepatocytes), muscle cells (cardiomyocytes, skeletal muscle cells, smooth muscle cells), retinal cells (ganglion cells, muller cells, photoreceptor cells), retinal pigment epithelial cells, renal cells (podocytes, proximal tubule cells, collecting duct cells, distal tubule cells), adrenal cells (cortical adrenal cells, medullary adrenal cells), pancreatic cells (alpha cells, beta cells, delta cells, epsilon cells, pancreatic polypeptide producing cells, exocrine cells); lung cells, bone marrow cells (early B-cell development, early T-cell development, macrophages, monocytes), urothelial cells, fibroblasts, parathyroid cells, thyroid cells, hypothalamic cells, pituitary cells, salivary gland cells, ovarian cells, and testicular cells.

Additional exemplary cell types that may comprise the recombinant genetic construct described herein include, without limitation, placental cells, keratinocytes, basal epidermal cells, urinary epithelial cells, salivary gland cells, mucous cells, serous cells, von Ebner's gland cells, mammary gland cells, lacrimal gland cells, eccrine sweat gland cells, apocrine sweat gland cells, MpH gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing P cells, glucagon-producing α cells, somatostatin-producing S cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, gonad cells, juxtaglomerular cells of the kidney, macula Densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair cells, cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fiber cells, melanocytes, retinal pigmented epithelial cells, iris pigment epithelial cells, oogonium, oocytes, spermatocytes, spermatogonium, ovarian cells, Sertoli cells, and thymus epithelial cells.

In accordance with this aspect of the disclosure, the recombinant genetic construct is integrated into the chromosome of the one or more cells in the preparation. The term “integrated,” when used in the context of the recombinant genetic construct of the present disclosure means that the recombinant genetic construct is inserted into the genome or the genomic sequence of the one or more cells in the preparation. When integrated, the integrated recombinant genetic construct is replicated and passed along to daughter cells of a dividing cell in the same manner as the original genome of the cell.

In accordance with the design of the recombinant genetic construct, the genomic integration of the construct is targeted to a desired gene of interest to achieve the cell selective expression of the one or more immune checkpoint protein encoding nucleotide sequences and/or the nucleotide sequence encoding one or more agents that reduce expression of the one or more HLA-I and/or HLA-II molecules. In some embodiments, the gene of interest is a gene restrictively expressed in a terminally differentiated cell. In some embodiments, the recombinant genetic construct is integrated into a gene selectively expressed in oligodendrocytes, such as SOX10, MYRF, MAG, or MBP. In some embodiments, the recombinant genetic construct is integrated into a gene selectively expressed in astrocytes, such as GFAP or AQP4. In some embodiments, the recombinant genetic construct is integrated into a gene selectively expressed in neurons, such as SYN1, MAP2, and ELAV4; a gene selectively expressed in dopaminergic neurons, such as TH or DDC; a gene selectively expressed in medium spiny neurons and interneurons, such as GAD65 or GAD67; or a gene selectively expressed in cholinergic neurons, such as CHAT. In accordance with these embodiments, the one or more immune checkpoint protein encoding nucleotide sequences and/or the nucleotide sequence encoding one or more agents that reduce expression of the one or more HLA-I and HLA-II molecules are conditionally expressed (i.e., transcribed and/or translated) in terminally differentiated cells. Expression of the recombinant genetic construct as described herein in a preparation of terminally differentiated cells renders those cells less susceptible to attack by immune cells in an in vivo environment. Thus, upon transplantation of cells comprising the recombinant genetic construct into a host subject, as described in more detail infra, the cells, in their differentiated state, are protected from attack by the host immune system as a result of their expression of one or more immune checkpoint proteins and/or expression of one or more agents that inhibit one or more HLA-I/HLA-II proteins.

Another aspect of the present disclosure relates to a method of administering a preparation of cells comprising the recombinant genetic construct as described herein to a subject in need thereof.

As used herein, a “subject” or a “patient” suitable for administering a preparation of cells comprising the recombinant genetic construct described herein encompasses any animal, preferably a mammal. Suitable subjects include, without limitation, domesticated and undomesticated animals such as rodents (mouse or rat), cats, dogs, rabbits, horses, sheep, pigs, and monkeys. In one embodiment the subject is a human subject. Suitable human subjects include, without limitation, infants, children, adults, and elderly subjects.

In one embodiment, the subject is in need of a terminally differentiated cell type. For example, the subject has a condition mediated by the loss of or dysfunction of a differentiated cell population. Thus, a cell preparation comprising the recombinant genetic construct is administered to such subject in an amount sufficient to restore normal levels and/or function of the differentiated cell population in the selected subject, thereby treating the condition. In some embodiments, the cell preparation comprising the recombinant genetic construct that is administered to the subject is a preparation of the differentiated cell population that is lost or dysfunctional in the subject. In another embodiment, the cell preparation comprising the recombinant genetic construction that is administered to the subject is a preparation of precursor or progenitor cells of the differentiated cell population. In accordance with this embodiment, the precursor or progenitors cells comprising the recombinant genetic construct mature or differentiate into the desired differentiated cell population after administration to the subject in need thereof.

In carrying out the methods of the present disclosure, “treating” or “treatment” includes inhibiting, preventing, ameliorating or delaying onset of a particular condition. Treating and treatment also encompasses any improvement in one or more symptoms of the condition or disorder. Treating and treatment encompasses any modification to the condition or course of disease progression as compared to the condition or disease in the absence of therapeutic intervention.

In some embodiments, the administering is effective to reduce at least one symptom of a disease or condition that is associated with the loss or dysfunction of the differentiated cell type. In another embodiment, the administering is effective to mediate an improvement in the disease or condition that is associated with the loss or dysfunction of the differentiated cell type. In another embodiment, the administering is effective to prolong survival in the subject as compared to expected survival if no administering were carried out.

In accordance with this aspect of the present disclosure, the preparation of one or more cells comprising the recombinant genetic construct may be autologous/autogenetic (“self”) to the recipient subject. In another embodiment, the preparation of cells comprising the recombinant genetic construct are non-autologous (“non-self,” e.g., allogeneic, syngeneic, or xenogeneic) to the recipient subject.

In carrying out the methods of the present disclosure, the administering may be carried out in the absence of immunosuppression or a modified course of immunosuppression therapy. For example, in one embodiment, the administering may be followed up with an initial course of immunosuppression therapy, but the administration of long-term immunosuppression therapy is not required.

In one embodiment, the method of treating a subject in need of a preparation of cells described herein involves treating a subject having a condition mediated by a loss or dysfunction of oligodendrocytes or by a loss or dysfunction of myelin, which is produced by oligodendrocytes. This method involves administering to the subject a preparation of cells comprising the recombinant genetic construct as described herein, where the preparation of cells is a preparation of glial progenitor cells or oligodendrocyte-biased progenitor cells. In accordance with this method, the cells are administered in an amount sufficient and under conditions effective to treat the condition mediated by the loss or dysfunction of oligodendrocytes or by the loss or dysfunction of myelin.

Oligodendrocytes produce myelin, an insulating sheath required for the salutatory conduction of electrical impulses along axons (Goldman et al., “How to Make an Oligodendrocyte,” Development 142(23):3983-3985 (2015), which is hereby incorporated by reference in its entirety). As described herein, oligodendrocyte loss results in demyelination, which leads to impaired neurological function in a broad array of disease ranging from pediatric leukodystrophies and cerebral palsy, to multiple sclerosis and white matter stroke.

Conditions mediated by a loss of myelin or by dysfunction or loss of oligodendrocytes that can be treated in accordance with the methods and cell preparations comprising the recombinant genetic construct as described herein include hypomyelination disorders and demyelinating disorders. In one embodiment, the condition is an autoimmune demyelination condition, such as e.g., multiple sclerosis, Schilder's Disease, neuromyelitis optica, transverse myelitis, and optic neuritis. In another embodiment, the myelin-related disorder is a vascular leukoencephalopathy, such as e.g., subcortical stroke, diabetic leukoencephalopathy, hypertensive leukoencephalopathy, age-related white matter disease, and spinal cord injury. In another embodiment, the myelin-related condition is a radiation induced demyelination condition. In another embodiment, the myelin-related disorder is a pediatric leukodystrophy, such as e.g., Pelizaeus-Merzbacher Disease, Tay-Sach Disease, Sandhoff's gangliosidosis, Krabbe's disease, metachromatic leukodystrophy, mucopolysaccharidoses (e.g., Sly's disease), Niemann-Pick A disease, adrenoleukodystrophy, Canavan's disease, Vanishing White Matter Disease, and Alexander Disease. In yet another embodiment, the myelin-related condition is periventricular leukomalacia or cerebral palsy.

Methods of generating glial progenitor cells or oligodendrocyte-biased progenitor cells suitable for treatment of a subject having a condition mediated by a loss or dysfunction of oligodendrocytes or myelin are known in the art, see e.g., U.S. Pat. No. 9,790,553 to Goldman et al., U.S. Pat. No. 10,190,095 to Goldman et al., and U.S. Patent Application Publication No. 2015/0352154 to Goldman et al., each of which are hereby incorporated by reference in their entirety. These cells are modified in accordance with the present disclosure to comprise the recombinant genetic vector at any point prior to transplantation. For example, in one embodiment, the recombinant genetic construct is introduced into the glial progenitor or oligodendrocyte-biased progenitor cells just prior to transplant. In another embodiment, the recombinant genetic construct is introduced into a precursor cell of the glial progenitor or oligodendrocyte-biased progenitor cells, e.g., neural progenitor cells or pluripotent stem cells.

In another embodiment, the method of treating a subject in need of a preparation of cells described herein involves treating a condition mediated by a loss or dysfunction of astrocytes. This method involves administering to the subject a preparation cells comprising the recombinant genetic construct as described herein, where the preparation of cells is a preparation of glial progenitor cells or astrocyte-biased progenitor cells. The cells are administered in an amount sufficient and under conditions effective to treat the condition mediated by the loss or dysfunction of astrocytes.

As described above, astrocytes are the largest and most prevalent type of glial cell in the central nervous system. Astrocytes contribute to formation of the blood-brain barrier, participate in the maintenance of extracellular ionic and chemical homeostasis, are involved in the response to injury, and affect neuronal development and plasticity.

Thus, in some embodiments, the condition mediated by a loss or dysfunction of astrocytes is a neurodegenerative disorder. Neurodegenerative disorders associated with a loss of astrocytes that can be treated in accordance with the methods and cell preparations of the present disclosure include, without limitation, Parkinson's Disease (PD), Alzheimer's disease (AD) and other dementias, degenerative nerve diseases, encephalitis, epilepsy, genetic brain disorders, head and brain malformations, hydrocephalus, multiple sclerosis, Amyotrophic Lateral Sclerosis (ALS or Lou Gehrig's Disease), Huntington's disease (HD), prion diseases, frontotemporal dementia, dementia with Lewy bodies, progressive supranuclear palsy, corticobasal degeneration, multiple system atrophy, hereditary spastic paraparesis, spinocerebellar atrophies, amyloidoses, motor neuron diseases (MND), spinocerebellar ataxia (SCA), and stroke and spinal muscular atrophy (SMA).

Methods of generating glial progenitor cells or astrocyte-biased progenitor cells suitable for treatment of a subject having a condition mediated by a loss or dysfunction of astrocytes are known in the art, see e.g., U.S. Patent Application Publication No. 2015/0352154 to Goldman et al., which is hereby incorporated by reference in its entirety. These cells are modified in accordance with the present disclosure to comprise the recombinant genetic vector at any point prior to transplantation into the subject in need thereof. For example, in one embodiment, the recombinant genetic construct is introduced into the glial progenitor or astrocyte-biased progenitor cells just prior to transplant. In another embodiment, the recombinant genetic construct is introduced into a precursor cell of the glial progenitor or astrocyte-biased progenitor cells, e.g., neural progenitor or pluripotent stem cells.

In another embodiment, the method of treating a subject in need of a preparation of cells described herein involves treating a condition mediated by a loss or dysfunction of neurons. This method involves administering to the subject a preparation cells comprising the recombinant genetic construct as described herein, where the preparation of cells is a preparation of neuronal progenitor cells. The cells are administered in an amount sufficient and under conditions effective to treat the condition mediated by the loss or dysfunction of neurons.

In accordance with this embodiment, the condition to be treated may be a condition mediated by the loss or dysfunction of a particular type of neuron. For example, in one embodiment the condition to be treated is a condition mediated by the loss or dysfunction of cholinergic neurons. Exemplary conditions mediated by the loss or dysfunction of cholinergic neurons include Alzheimer's disease, corticobasal degeneration, dementia with Lewy bodies, frontotemporal dementia, multiple system atrophy, Parkinson's disease, Parkinson's disease dementia, and progressive supranuclear palsy (Roy et al., “Cholinergic Imaging in Dementia Spectrum Disorders,” Eur. J. Nucl. Med. Mol. Imaging. 43:1376-1386 (2016), which is hereby incorporated by reference in its entirety).

In another embodiment, the conditions to be treated is a condition mediated by the loss or dysfunction of dopaminergic neurons. Exemplary conditions mediated by the loss or dysfunction of dopaminergic neurons include Parkinson's disease, Parkinsonian-like disorders (e.g., juvenile parkinsonism, Ramsey-Hunt paralysis syndrome), and mental disorders (e.g., schizophrenia, depression, drug addiction).

In another embodiment, the condition to be treated is a condition mediated by the loss or dysfunction of medium spiny neurons and/or cortical interneurons. Exemplary conditions mediated by the loss or dysfunction of medium spiny neurons and/or cortical interneurons include Huntington's disease, epilepsy, anxiety, and depression (Powell et al., “Genetic Disruption of Cortical Interneuron Development Causes Region- and GABA Cell Type-Specific Deficits, Epilepsy, and Behavioral Dysfunction,” J. Neurosci. 23(2):622-631 (2003), which is hereby incorporated by reference in its entirety).

Methods of generating neuronal progenitor cells suitable for treatment of a subject having a condition mediated by a loss or dysfunction of neurons are known in the art, see e.g., Goldman, SAl., “Transplanted Neural Progenitors Bridge Gaps to Benefit Cord-Injured Monkeys.” Nat. Med. 24(4):388-390 (2018); Roy et al., “Functional Engraftment of Human ES Cell-Derived Dopaminergic Neurons Enriched by Coculture with Telomerase-Immortalized Midbrain Astrocytes,” Nat. Med. 12(11):1259-1268 (2006); Nunes et al., “Identification and Isolation of Multipotential Neural Progenitor Cells from the Subcortical White Matter of the Adult Human Brain,” Nat. Med. 9(4):439-447 (2003), U.S. Pat. No. 6,812,027 to Goldman et al.; U.S. Pat. No. 7,150,989 to Goldman et al.; U.S. Pat. No. 7,468,277 to Goldman et al.; U.S. Pat. No. 7,785,882 to Goldman; U.S. Pat. No. 8,263,406 to Goldman et al.; U.S. Pat. No. 8,642,332 to Goldman et al.; and U.S. Pat. No. 8,945,921 to Goldman et al., each of which is hereby incorporated by reference in its entirety. These cells are modified in accordance with the present disclosure to comprise the recombinant genetic vector at any point prior to transplantation into the subject in need thereof. For example, in one embodiment, the recombinant genetic construct is introduced into the neuronal progenitor cells just prior to transplant. In another embodiment, the recombinant genetic construct is introduced into a precursor cell of the neuronal progenitor cells, e.g., neural progenitor or pluripotent stem cells.

In carrying out the methods of the present invention involving cell replacement in central nervous system, the preparation of cells described herein can be administered systemically into the circulation, or administered directly to one or more sites of the brain, the brain stem, the spinal cord, or a combination thereof.

When the preparation of cells is injected systemically into the circulation, the preparation of cells may be placed in a syringe, cannula, or other injection apparatus for precise placement at a preselected site. The term “injectable” means the preparation of cells can be dispensed from syringes under normal conditions under normal pressure.

Methods for direct administration of (i.e., transplanting) various nerve tissues/cells into a host brain are well known in the art. In some embodiments, the preparation is administered intraventricularly, intracallosally, or intraparenchymally.

Intraparenchymal administration, i.e. within the host brain (as compared to outside the brain or extraparenchymal transplantation) is achieved by injection or deposition of cells within the brain parenchyma at the time of administration. Intraparenchymal transplantation can be performed using two approaches: (i) injection of the preparation of cells into the host brain parenchyma or (ii) preparing a cavity by surgical means to expose the host brain parenchyma and then depositing the preparation of cells into the cavity. Both methods provide parenchymal deposition between the preparation of cells and the host brain tissue at the time of administration, and both facilitate anatomical integration between the graft (i.e., the preparation of cells) and the host brain tissue.

Alternatively, the cell graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura.

For transplantation into cavities, which may be preferred for spinal cord grafting, tissue is removed from regions close to the external surface of the CNS to form a transplantation cavity, by removing bone overlying the brain and stopping bleeding with a material such a gelfoam. Suction may be used to create the cavity. The preparation of cells is then placed in the cavity. More than one preparation of cells may be placed in the same cavity. In some embodiments, the site of implantation is dictated by the CNS disorder being treated.

Injections into selected regions of the host brain may be made by drilling a hole and piercing the dura to permit the needle of a microsyringe to be inserted. The microsyringe is preferably mounted in a stereotaxic frame and three dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

The number of cells in a given volume can be determined by well-known and routine procedures and instrumentation. The percentage of the cells in a given volume of a mixture of cells can be determined by much the same procedures. Cells can be readily counted manually or by using an automatic cell counter. Specific cells can be determined in a given volume using specific staining and visual examination and by automated methods using specific binding reagent, typically antibodies, fluorescent tags, and a fluorescence activated cell sorter.

The preparation of cells can be administered in dosages and by techniques well known to those skilled in the medical and veterinary arts taking into consideration such factors as the age, sex, weight, and condition of the particular patient, and the formulation that will be administered. The dose appropriate to be used in accordance with various embodiments described herein will depend on numerous factors. It may vary considerably for different circumstances. The parameters that will determine optimal doses to be administered for primary and adjunctive therapy generally will include some or all of the following: the disease being treated and its stage; the species of the subject, their health, gender, age, weight; the subject's immunocompetence; other therapies being administered; and expected potential complications from the subject's history or genotype. The parameters may also include: whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the cells/medium are formulated, the way they are administered, and the degree to which the cells/medium will be localized at the target sites following administration. Finally, the determination of optimal dosing necessarily will provide an effective dose that is neither below the threshold of maximal beneficial effect nor above the threshold where the deleterious effects associated with the dose outweighs the advantages of the increased

For fairly pure preparations of cells, optimal doses in various embodiments will range from about 10⁴ to about 10⁹ cells per administration. In some embodiments, the optimal dose per administration will be between about 10⁵ to about 10⁷ cells. In many embodiments the optimal dose per administration will be about 5×10⁵ to about 5×10⁶ cells.

It is to be appreciated that a single dose may be delivered all at once, fractionally, or continuously over a period of time. The entire dose also may be delivered to a single location or spread fractionally over several locations.

Human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regimens can be ascertained by the skilled artisan, from this disclosure, the documents cited herein, and the knowledge in the art.

In some embodiments, the preparation of cells is administered to a subject in one dose. In others, the preparation of cells is administered to a subject in a series of two or more doses in succession. In some other embodiments where the preparation of cells is administered in a single dose, in two doses, and/or more than two doses, the doses may be the same or different, and they are administered with equal or with unequal intervals between them.

The preparation of cells may be administered in many frequencies over a wide range of times. In some embodiments, they are administered over a period of less than one day. In other embodiments, they are administered over two, three, four, five, or six days. In some embodiments, they are administered one or more times per week, over a period of weeks. In other embodiments, they are administered over a period of weeks for one to several months. In various embodiments, they may be administered over a period of months. In others they may be administered over a period of one or more years. Generally, lengths of treatment will be proportional to the length of the disease process, the effectiveness of the therapies being applied, and the condition and response of the subject being treated.

The choice of formulation for administering the composition for a given application will depend on a variety of factors. Prominent among these will be the species of subject, the nature of the disorder, dysfunction, or disease being treated and its state and distribution in the subject, the nature of other therapies and agents that are being administered, the optimum route for administration, survivability via the route, the dosing regimen, and other factors that will be apparent to those skilled in the art. In particular, for instance, the choice of suitable carriers and other additives will depend on the exact route of administration and the nature of the particular dosage form.

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic cells. Thus, measures may be taken to increase cell survival and/or to overcome problems posed by barriers to seeding and/or growth.

Final formulations may include an aqueous suspension of cells/medium and, optionally, protein and/or small molecules, and will typically involve adjusting the ionic strength of the suspension to isotonicity (i.e., about 0.1 to 0.2) and to physiological pH (i.e., about pH 6.8 to 7.5). The final formulation will also typically contain a fluid lubricant, such as maltose, which must be tolerated by the body. Exemplary lubricant components include glycerol, glycogen, maltose, and the like. Organic polymer base materials, such as polyethylene glycol and hyaluronic acid as well as non-fibrillar collagen, such as succinylated collagen, can also act as lubricants. Such lubricants are generally used to improve the injectability, intrudability, and dispersion of the injected material at the site of injection and to decrease the amount of spiking by modifying the viscosity of the compositions. This final formulation is by definition the cells described herein in a pharmaceutically acceptable carrier.

Multiple preparations of cells may be administered concomitantly to different locations such as combined administration intrathecally and intravenously to maximize the chance of targeting into affected areas.

An additional aspect relates to a preparation of one or more cells, where cells of the preparation are modified to conditionally express increased levels of one or more immune checkpoint proteins as compared to a corresponding wild-type cell. In one embodiment, the cells of the preparation are further modified to conditionally express reduced levels of one or more endogenous HLA-I proteins as compared to a corresponding wild-type cell. In some embodiments, the cells of the preparation are further modified to conditionally express reduced levels of one or more HLA-II proteins as compared to corresponding wild-type cells.

Another aspect relates to a preparation of one or more cells, where cells of the preparation are modified to conditionally express reduced levels of one or more endogenous HLA-I proteins as compared to a corresponding wild-type cell. In some embodiments, the cells of the preparation are further modified to conditionally express reduced levels of one or more HLA-II proteins as compared to corresponding wild-type cells.

Exemplary immune checkpoint proteins to be conditionally expressed in the modified cells of the preparation are described in detail supra, and include, e.g., programmed death ligand 1 (PD-L1), programmed death ligand 2 (PD-L2), CD47, HLA-E, CD200, and CTLA-4.

Likewise, exemplary HLA-I proteins, whose expression is conditionally reduced in the modified cells of the preparation are described supra, and include, e.g., one or more of HLA-A, HLA-B, HLA-C, HLA-E, HLA-F, HLA-G, and combinations thereof. Exemplary HLA-II proteins whose expression is conditionally reduced in the modified cells of the preparation include any one or more of HLA-DM, HLA-DO, HLA-DP, HLA-DQ, HLA-DR.

Yet another aspect of the present disclosure relates to a method of generating a conditionally immunoprotected cell. This method involves modifying a cell to (i) conditionally express increased levels of one or more immune checkpoint proteins or (ii) conditionally express one or more agents that reduce surface expression of one or more endogenous HLA-proteins. In another embodiment, the method involves modifying a cell to (i) conditionally express increased levels of one or more immune checkpoint proteins and (ii) conditionally express one or more agents that reduce surface expression of one or more endogenous HLA-proteins.

In accordance with this aspect of the disclosure, the conditional expression of the one or more immune checkpoint proteins and/or the conditional expression of the one or more agents that reduce expression of one or more endogenous HLA proteins is operably linked to the expression of a gene that is restrictively expressed in a terminally differentiated cell. Suitable terminally differentiated cells and genes selectively expressed therein are described in detail supra.

Cells that can be modified in accordance with this aspect of the disclosure include cells from any organism. In some embodiments, the preparation is a preparation of mammalian cells, e.g., a preparation of rodent cells (i.e., mouse or rat cells), rabbit cells, guinea pig cells, feline cells, canine cells, porcine cells, equine cells, bovine cell, ovine cells, monkey cells, or human cells. Suitable cells include primary or immortalized embryonic cells, fetal cells, or adult cells, at any stage of their lineage, e.g., totipotent, pluripotent, multipotent, or differentiated cells.

In some embodiments, modifying the cells of interest involves introducing into the cell a sequence-specific nuclease that cleaves a target gene at or within the gene's 3′ UTR, or a position just upstream of the 3′ UTR. As described in detail supra, a suitable target gene is a gene that is selectively or restrictively expressed in a cell specific manner. Once the target gene is cleaved by a sequence-specific nuclease, the method further involves introducing into the target gene, for example, by way of homologous recombination, any of the recombinant genetic constructs described herein.

Suitable sequence specific nucleases for cleaving the target gene to introduce the recombinant genetic construct include, without limitation, zinc finger nucleases (ZFN), transcription activator-like effector nucleases (TALEN), and an RNA-guided nucleases. In some embodiments, the sequence-specific nuclease is introduced into the cell as a protein, mRNA, or cDNA.

Zinc finger nucleases are a class of engineered DNA binding proteins that facilitate targeted editing of DNA by introducing double strand DNA breaks in a sequence specific manner. Each ZFN comprises two functional domains, i.e., a DNA-binding domain comprised of α chain of two-finger modules, each recognizing a unique hexamer sequence of DNA, and a DNA-cleaving domain comprised of the nuclease domain of Fok I. ZFNs suitable for targeted cleavage of the target genes described herein to facilitate insertion of the recombinant genetic construct are known in the art, see e.g., U.S. Pat. No. 8,106,255 to Carroll et al., U.S. Pat. No. 9,428,756 to Cai et al., U.S. Patent Publication No. 20110281306 to Soo and Joo; U.S. Patent Publication No. 20050130304 to Cox et al., which are hereby incorporated by reference in their entirety.

In another embodiment transcription activator-like effector nuclease (TALEN)-mediated DNA editing is utilized to introduce the recombinant genetic construct described herein into a target gene of interest. A functional TALEN consists of a DNA binding domain, which is derived from transcription activator-like effector (TALE) proteins, and a nuclease catalytic domain from a DNA nuclease, FokI. The DNA binding domain of TALE features an array of 33-34 amino acid repeats. Each repeat is conserved, with the exception of the repeat variable di-residues (RVDs) at amino acid positions 12 and 13, which determine which nucleotide of the targeted DNA sequence each repeat recognizes. Methods of customizing TALE proteins to bind to a target site using canonical or non-canonical RVDs within the repeat units are known in the art and suitable for use in accordance with the present disclosure (see, e.g., U.S. Pat. No. 8,586,526 to Philip et al. and U.S. Pat. No. 9,458,205 to Philip et al., which are hereby incorporated by reference in their entirety). Likewise, methods of using TALEN for gene editing that are suitable for use in accordance with the present disclosure are also known in the art, see e.g., U.S. Pat. No. 9,393,257 to Osborn et al., which is hereby incorporated by reference in its entirety.

In another embodiment, the sequence specific nuclease used to introduce the recombinant genetic construct described herein into a target gene of interest is an RNA-guided nuclease in the form of Cas9. Cas9 is a CRISPR-associated protein containing two nuclease domains, that, when complexed with CRISPR RNA (cRNA) and trans-activating rRNA, can achieve site-specific DNA recognition and double strand cleavage. CRISPR-Cas9 systems and methods for gene editing that are suitable for use in accordance with the present disclosure are well known in the art, see, e.g., Jinek, M., et al. “A Programmable Dual-RNA-Guided DNA Endonuclease in Adaptive Bacterial Immunity,” Science 337:816-821 (2012); Doench et al., “Rational Design of Highly Active sgRNAs for CRISPR-mediated Gene Inactivation,” Nature Biotechnol. 32(12): 1262-7 (2014) U.S. Pat. No. 9,970,001 to Miller; U.S. Patent Publication No. 20180282762 to Gori et al., and U.S. Patent Publication No. 20160201089 to Gersbach et al., which are hereby incorporated by reference in their entirety.

### EXAMPLES

The following examples are provided to illustrate embodiments of the present invention but they are by no means intended to limit its scope.

### Example 1—Recombinant Genetic Knock-In Constructs for Targeted Expression in Terminally Differentiated Cells

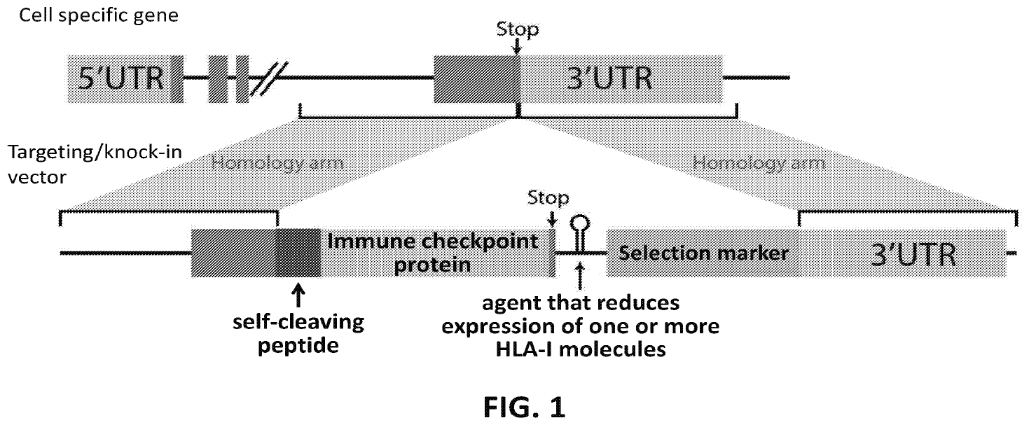

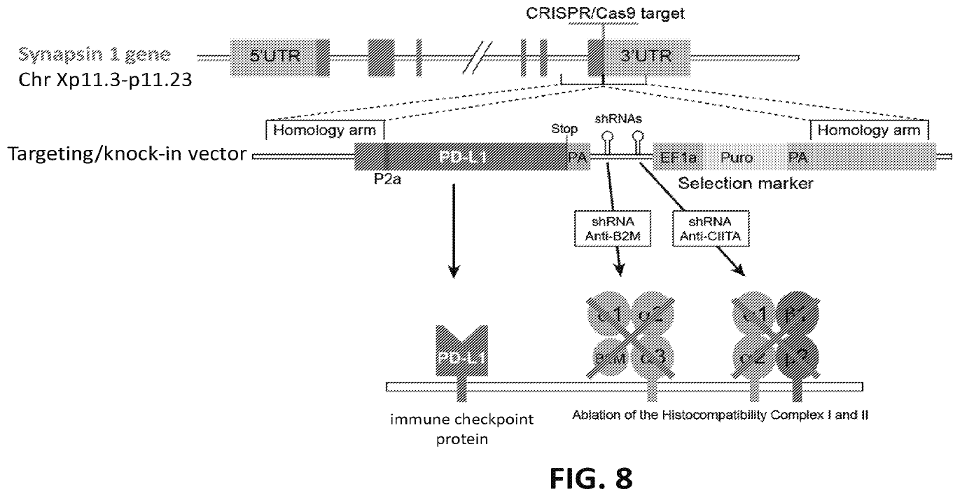

The design of various recombinant genetic constructs comprising an immune-inhibitory protein knock-in vector targeting a cell specific gene (e.g., MYRF, SYN1, or GFAP) is shown in FIGS. 1-14.

FIG. 1 shows the general design for a recombinant genetic construct comprising a first gene sequence expressed in a cell-type specific manner (i.e., a 5′ homology arm), a self-cleaving peptide encoding nucleotide sequence (e.g., P2a), first nucleotide sequence encoding one or more immune-inhibitory proteins (e.g., HLA-E/syB2M, CD47, or PD-L1), a stop codon, second nucleotide sequence encoding one or more agents that reduce surface expression of one or more endogenous HLA-I molecules (i.e., an shRNA), a selection marker, and a second gene sequence expressed in a cell-type specific manner (i.e., a 3′ homology arm).

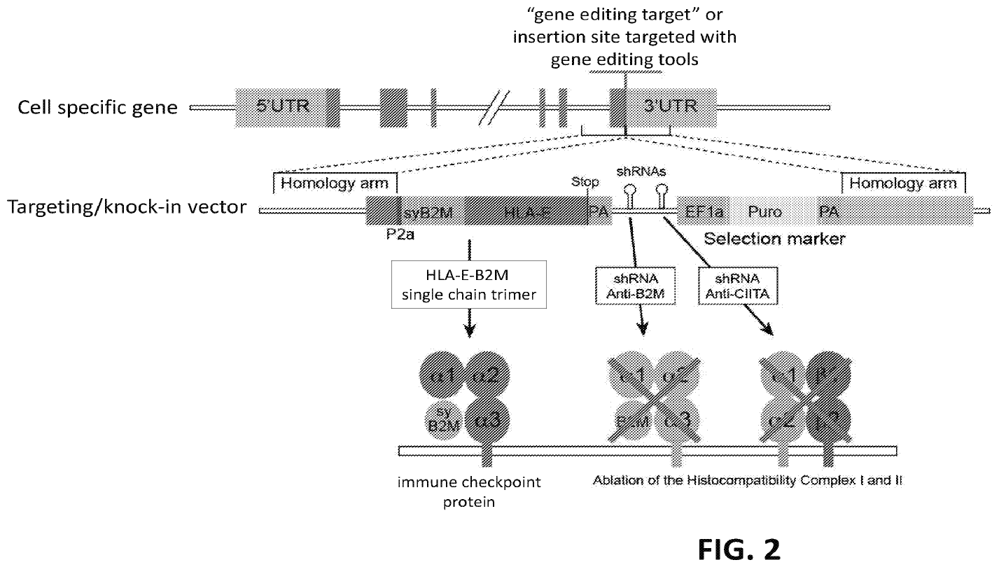

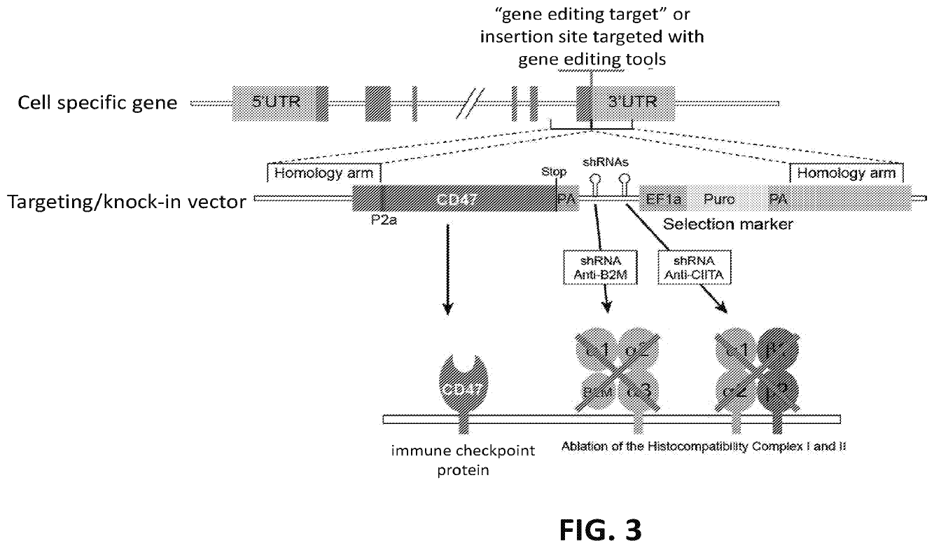

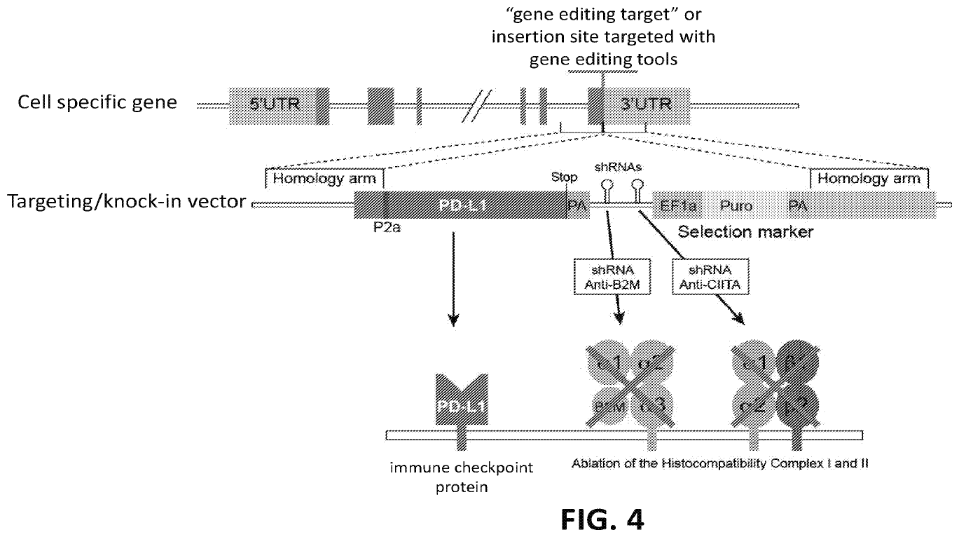

FIGS. 2-4 show the general design of knock-in vectors comprising a 5′ homology arm and 3′ homology arm. The knock in vectors encode an immune-inhibitory protein i.e., HLA-E/syB2M (FIG. 2), CD47 (FIG. 3), or PD-L1 (FIG. 4), a self-cleaving peptide (P2a), HLA-E/syB2M, an anti-B2M shRNA, an anti-CIITA shRNA, and puromycin. The expression of puromycin is operatively linked to EF1a promoter for constitutive expression in mammalian cells.

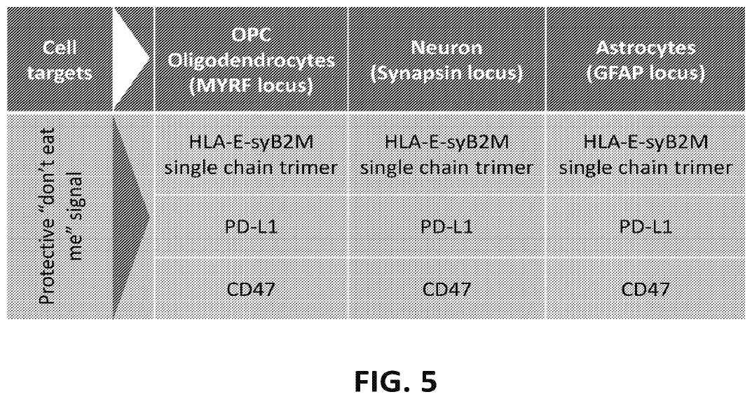

FIG. 5 is a matrix showing combinations of various target cells and protective signals (i.e., immune-inhibitory proteins or peptides thereof).

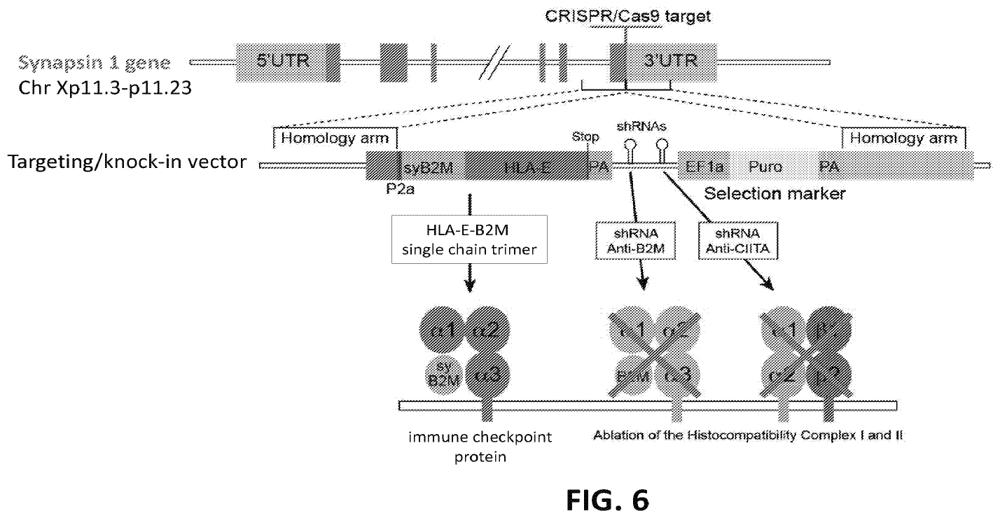

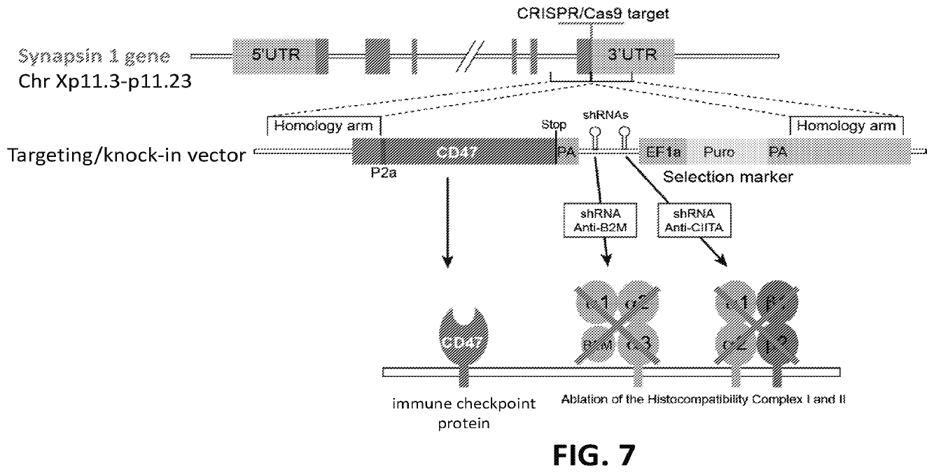

FIGS. 6-8 show the general exemplary design of knock-in vectors targeting the SYN1 gene locus to achieve expression in a neuron specific manner. Each SYN1-targeted knock-in vector comprises a 5′ homology arm and 3′ homology arm and encodes an immune-inhibitory protein i.e., HLA-E/syB2M (FIG. 6), CD47 (FIG. 7), or PD-L1 (IG. 8), a self-cleaving peptide (P2a), HLA-E/syB2M, an anti-B2M shRNA, an anti-CIITA shRNA, and puromycin. The expression of puromycin is operatively linked to EF1a promoter for constitutive expression in mammalian cells.

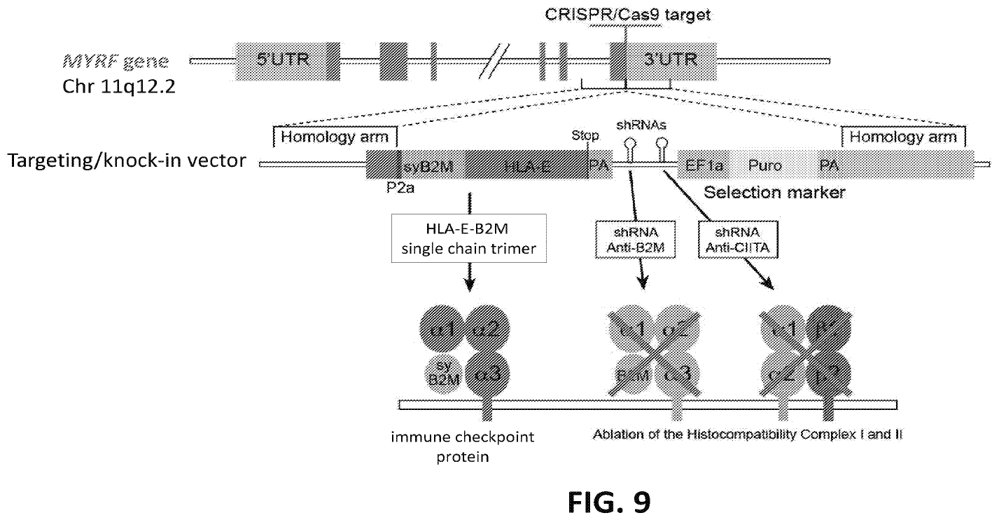

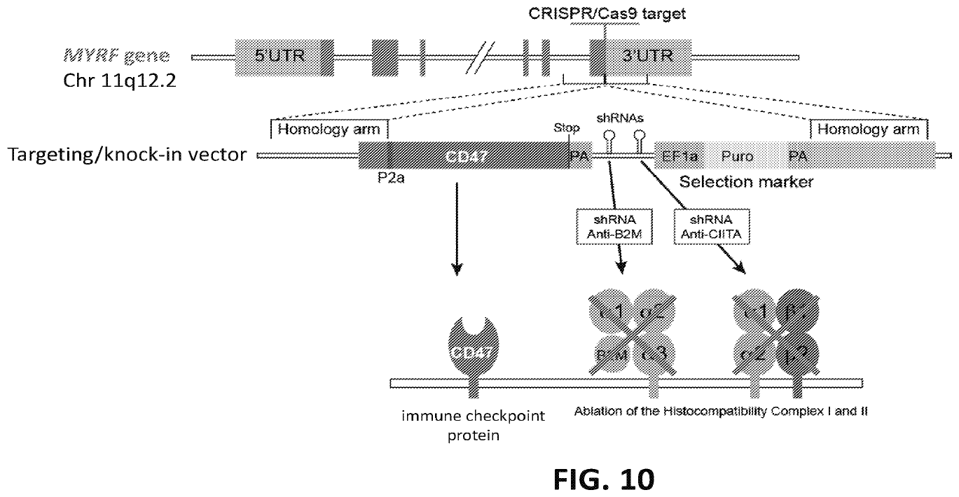

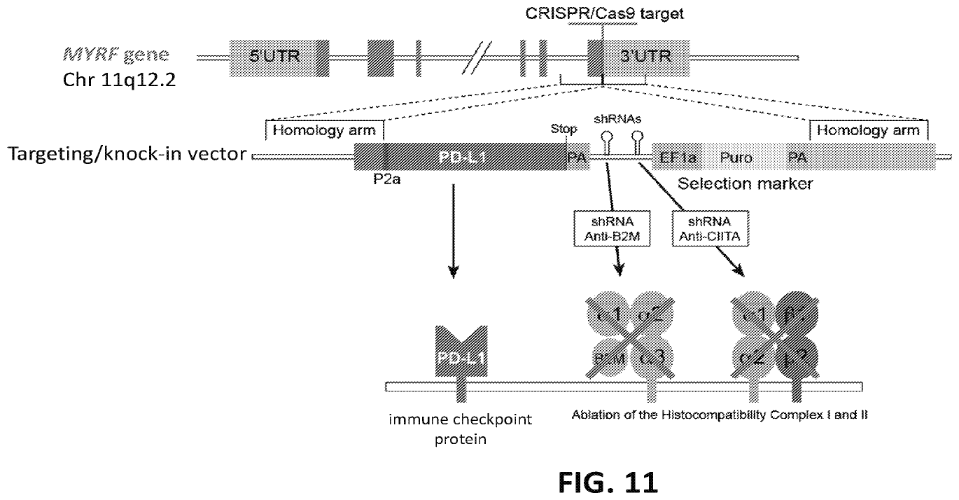

FIGS. 9-11 show the general design of knock-in vectors targeting the MYRF gene locus to achieve expression in an oligodendrocyte specific manner. Each MYRF-targeted knock-in vector comprises a 5′ homology arm and 3′ homology arm and encodes an immune-inhibitory protein i.e., HLA-E/syB2M (FIG. 9), CD47 (FIG. 10), or PD-L1 (FIG. 11), a self-cleaving peptide (P2a), HLA-E/syB2M, an anti-B2M shRNA, an anti-CIITA shRNA, and puromycin. The expression of puromycin is operatively linked to EF1a promoter for constitutive expression in mammalian cells.

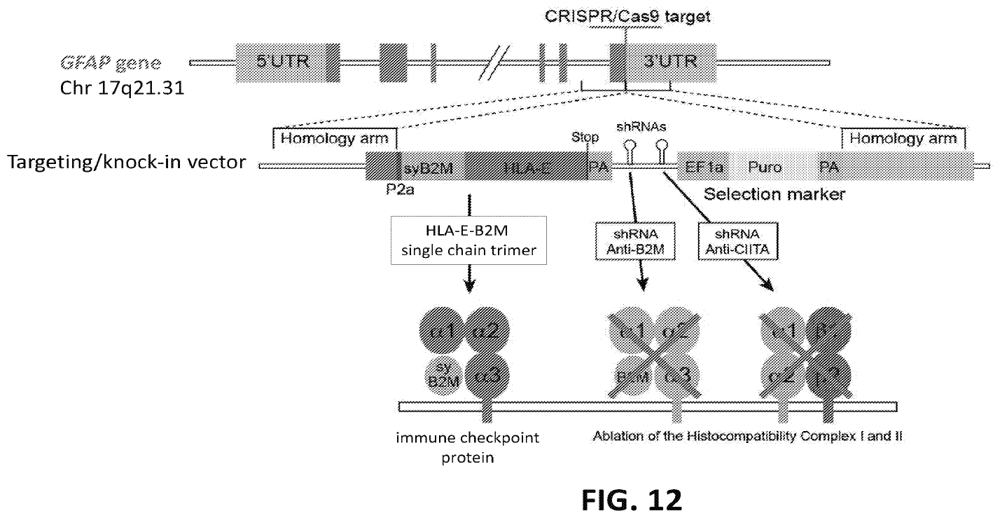

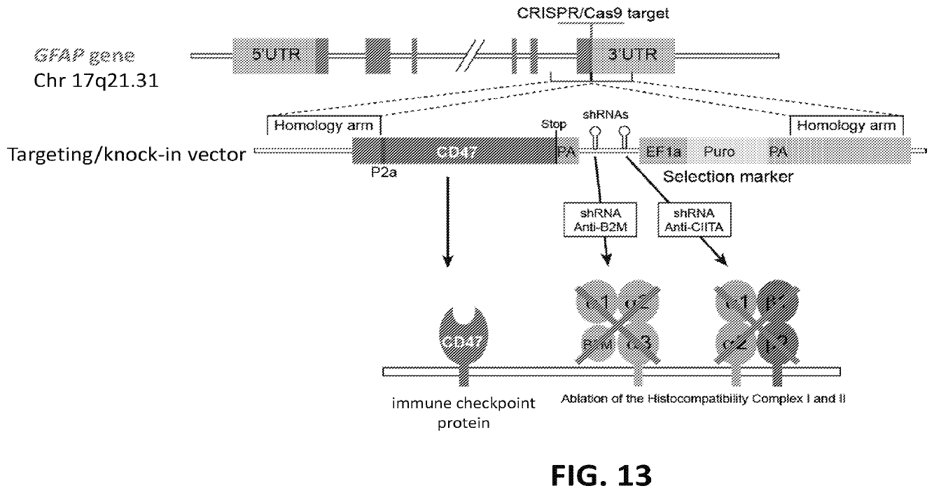

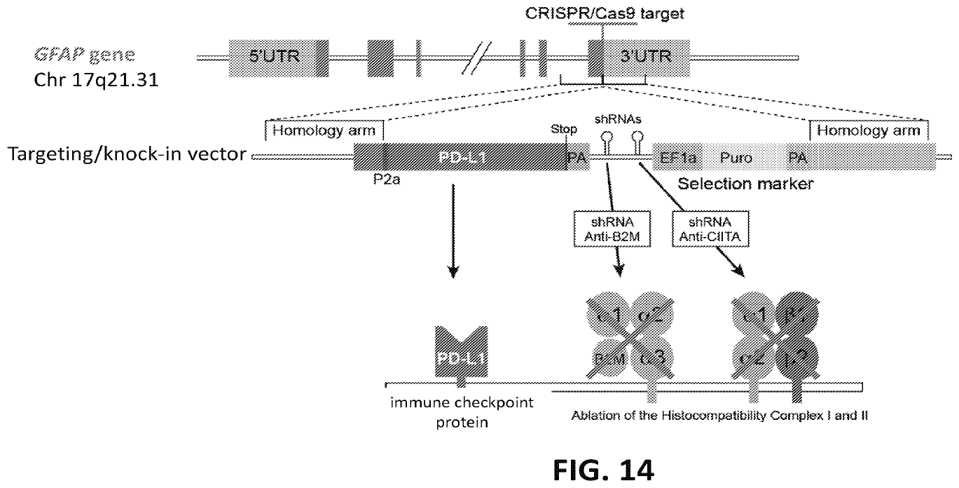

FIGS. 12-14 show the general design of knock-in vectors targeting the GFAP gene locus to achieve expression in an astrocyte specific manner. Each GFAP-targeted knock-in vector comprises a 5′ homology arm and 3′ homology arm and encodes an immune-inhibitory protein i.e., HLA-E/syB2M (FIG. 12), CD47 (FIG. 13), or PD-L1 (FIG. 14), a self-cleaving peptide (P2a), HLA-E/syB2M, an anti-B2M shRNA, an anti-CIITA shRNA, and puromycin. The expression of puromycin is operatively linked to EF1a promoter for constitutive expression in mammalian cells.

### Prophetic Example 2—Generation of a Recombinant Genetic Knock-In Construct Expressing CD47 cDNA with Target Sequences for the MYRF Locus

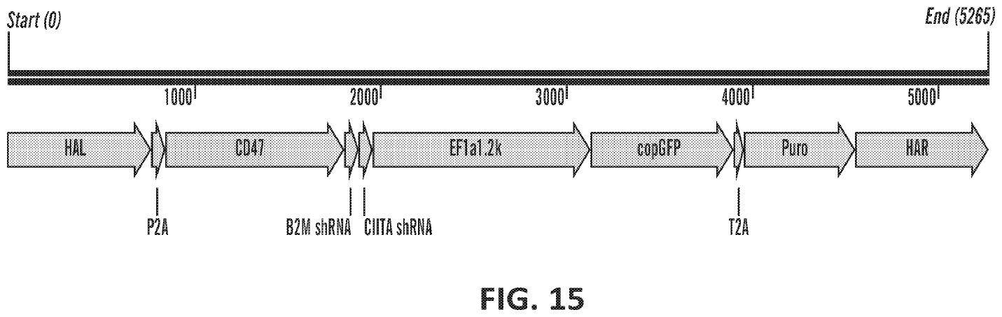

A schematic illustration of a recombinant genetic construct comprising a CD47 knock-in vector targeting the MYRF gene locus is shown in FIG. 15. The recombinant genetic construct comprises a 5′ homology arm (HAL), a self-cleaving peptide encoding nucleotide sequence (P2A), first nucleotide sequence encoding CD47, a second nucleotide sequence encoding anti-β₂M shRNA, a third nucleotide sequence encoding anti-CIITA shRNA, a nucleotide sequence encoding GFP operatively linked to the EF1a promoter, and a 3′ homology arm (HAR). The recombinant genetic construct of FIG. 15 will be produced as follows.

### β₂-Microglobulin and CIITA Knockdowns

shRNA for β₂M and CIITA will be generated using online tools (e.g., iRNA designer from Thermofisher). shRNA will be inserted immediately downstream of puromycin gene in lentiviral vector pTANK-EF1a-copGFP-Puro-WPRE. Virus particles pseudotyped with vesicular stomatitis virus G glycoprotein will be produced, concentrated by ultracentrifugation, and titrated on 293HEK cells.

HAD100-derived hGPCs will be transduced with lentivirus bearing shRNA for β₂M or CIITA (MOI=1). The efficiency of the knockdown will be evaluated by QPCR. shRNA with Knock downs efficiency >80% will be further validated by the expression of respective protein by immunostaining and western blot.

sgRNA Design and CRSPR/Cas9 Vector Construct

Single-guide RNAs will be designed to allow double nicking using the CRISPR/Cas9 design tool developed by the Zhang lab at MIT (crispr.mit.edu). sgRNA will be selected in the coding sequence right before the codon stop (e.g., TCAGGCCAACTGCAGTTCAGAGG (SEQ ID NO: 45)). sgRNA will be validated by transfection of HEK-29 cells using the Surveyor Mutation Detection Kits (IDT inc).

### Cloning of Homology Arms

Genomic DNA from the cells will be extracted using DNeasy Blood and Tissue Kit (QIAGEN) following to the manufacturer's instruction. AmpliTaq Gold 360 (Thermo Fisher Scientific) will be used to amplify homology arm from genomic DNA of HAD100 cell line (Primers TBD). Both homology arms will be subcloned into pCR2.1-TOPO and sequence validated. The Left homology arm (HAL) will include the last exon in the target gene.

hESCs Transfection and Selection

Knock-in and sgRNA-CRIPR/Cas9 plasmids will be amplified with Endotoxin free Maxi-prep kit (Qiagen). Both plasmid (3 μg each) will transfected into hESCs (800,000 cells) using the Amaxa 4D-Nucleofector (Lonza; program CA-137 was used as per the manufacturer's instructions). Twenty-four hours after electroporation, the cells will be grown in puromycin (1 μg/mL) containing media.

Singles colonies will be isolated and expanded. Transgenic clones will be validated by PCR for both correct integration of knock-in cassette and for the absence of sgRNA-CRISPR/Cas9 plasmid.

Suitable sequences for the generation of recombinant genetic knock-in constructs expressing CD47 cDNA with target sequences for the MYRF locus are shown in Table 14 below.

TABLE 14
Exemplary Sequences for a Recombinant Genetic Knock-In Construct
Expressing CD47 cDNA with Target Sequences for the MYRF Locus

| | | SEQ |
|---|---|---|
| Name | Nucleotide Sequence | ID NO. |
| MYRF | GGTTTGAATCCCAGCTGTGTGATTTTGCCACACTGTGTGATTTTTA | 46 |
| Right | GGAAGTGGCTCAGTTTCCTCATCCAGAAGATGGGGCTAGTAGCAGC | |
| Homology | ACTGTGTCACTGGATTGTACTGAGGATGGGGCTAATGAAATACTTT | |
| Arm | GATGTGCCCAGAGCATAGTGGGTGAGGGAACCCAGCACAACAGGAC | |
| | TGGGAAGGAGGCAGGGGCCAGGTGGAGGTGGCTGTGGACCTGCCAG | |
| | TCCCGGGCACGGTCTGCATGGAGTAGCTGCCATTGCTCCTTCTGCC | |
| | AAAGCAGAACATGCTCCTTCCTATCTCTTCAAAGTTCTCTGCTTTT | |
| | TTCCTTCATAAAACTCCCCACAGACCCCAGGACTGCGACGGCCGTG | |
| | GTGAGAGATGCTGGTTGGGATAAGGGCAGCAGTCTGTCCTGACCCC | |
| | TCTCTCCCTTCTCTCCAGGGCACCTCTCACCGGTGGCCAATAACCA | |
| | TCCTGTCCTTCCGTGAATTCACCTACCACTTCCGGGTGGCACTGCT | |
| | GGTGAGCAGGGGCATCCCACCTACCCTGGAGGTCTGGGCACCCCTG | |
| | TCTGCGACGTGGGGCTTGAGGAATGGGGGGTTTGCACAGTATGTGG | |
| | TAGGGCTGGGGGCACAGTGTCAAGCAATGTCAGCAGGGAGTGCCAT | |
| | CTGCCCCGCACCCCCAGAGCCACCTCACCTTCCCACTGCCCTTCCA | |
| | CCCAGGGTCAGGCCAACTGCAGT | |
| P2A | GGAAGCGGAGCTACTAACTTCAGCCTGCTGAAGCAGGCTGGAGACG | 47 |
| | TGGAGGAGAACCCTGGACCT | |
| Human | ATGTGGCCCCTGGTAGCGGCGCTGTTGCTGGGCTCGGCGTGCTGCG | 48 |
| CD47 | GATCAGCTCAGCTACTATTTAATAAAACAAAATCTGTAGAATTCAC | |
| (NM_0017 | GTTTTGTAATGACACTGTCGTCATTCCATGCTTTGTTACTAATATG | |
| 77.3) | GAGGCACAAAACACTACTGAAGTATACGTAAAGTGGAAATTTAAAG | |
| | GAAGAGATATTTACACCTTTGATGGAGCTCTAAACAAGTCCACTGT | |
| | CCCCACTGACTTTAGTAGTGCAAAAATTGAAGTCTCACAATTACTA | |
| | AAAGGAGATGCCTCTTTGAAGATGGATAAGAGTGATGCTGTCTCAC | |
| | ACACAGGAAACTACACTTGTGAAGTAACAGAATTAACCAGAGAAGG | |
| | TGAAACGATCATCGAGCTAAAATATCGTGTTGTTTCATGGTTTTCT | |
| | CCAAATGAAAATATTCTTATTGTTATTTTCCCAATTTTTGCTATAC | |
| | TCCTGTTCTGGGGACAGTTTGGTATTAAAACACTTAAATATAGATC | |
| | CGGTGGTATGGATGAGAAAACAATTGCTTTACTTGTTGCTGGACTA | |
| | GTGATCACTGTCATTGTCATTGTTGGAGCCATTCTTTTCGTCCCAG | |
| | GTGAATATTCATTAAAGAATGCTACTGGCCTTGGTTTAATTGTGAC | |
| | TTCTACAGGGATATTAATATTACTTCACTACTATGTGTTTAGTACA | |
| | GCGATTGGATTAACCTCCTTCGTCATTGCCATATTGGTTATTCAGG | |
| | TGATAGCCTATATCCTCGCTGTGGTTGGACTGAGTCTCTGTATTGC | |
| | GGCGTGTATACCAATGCATGGCCCTCTTCTGATTTCAGGTTTGAGT | |
| | ATCTTAGCTCTAGCACAATTACTTGGACTAGTTTATATGAAATTTG | |
| | TGGCTTCCAATCAGAAGACTATACAACCTCCTAGGAAAGCTGTAGA | |
| | GGAACCCCTTAATGCATTCAAAGAATCAAAAGGAATGATGAATGAT | |
| | GAATAA | |
| EF1a | GCTCCGGTGCCCGTCAGTGGGCAGAGCGCACATCGCCCACAGTCCC | 49 |
| Promoter | CGAGAAGTTGGGGGGAGGGGTCGGCAATTGAACCGGTGCCTAGAGA | |
| | AGGTGGCGCGGGGTAAACTGGGAAAGTGATGTCGTGTACTGGCTCC | |
| | GCCTTTTTCCCGAGGGTGGGGGAGAACCGTATATAAGTGCAGTAGT | |
| | CGCCGTGAACGTTCTTTTTCGCAACGGGTTTGCCGCCAGAACACAG | |
| | GTAAGTGCCGTGTGTGGTTCCCGCGGGCCTGGCCTCTTTACGGGTT | |
| | ATGGCCCTTGCGTGCCTTGAATTACTTCCACCTGGCTGCAGTACGT | |
| | GATTCTTGATCCCGAGCTTCGGGTTGGAAGTGGGTGGGAGAGTTCG | |
| | AGGCCTTGCGCTTAAGGAGCCCCTTCGCCTCGTGCTTGAGTTGAGG | |
| | CCTGGCCTGGGCGCTGGGGCCGCCGCGTGCGAATCTGGTGGCACCT | |
| | TCGCGCCTGTCTCGCTGCTTTCGATAAGTCTCTAGCCATTTAAAAT | |
| | TTTTGATGACCTGCTGCGACGCTTTTTTTCTGGCAAGATAGTCTTG | |
| | TAAATGCGGGCCAAGATCTGCACACTGGTATTTCGGTTTTTGGGGC | |
| | CGCGGGCGGCGACGGGGCCCGTGCGTCCCAGCGCACATGTTCGGCG | |
| | AGGCGGGGCCTGCGAGCGCGGCCACCGAGAATCGGACGGGGGTAGT | |
| | CTCAAGCTGGCCGGCCTGCTCTGGTGCCTGGCCTCGCGCCGCCGTG | |
| | TATCGCCCCGCCCTGGGCGGCAAGGCTGGCCCGGTCGGCACCAGTT | |
| | GCGTGAGCGGAAAGATGGCCGCTTCCCGGCCCTGCTGCAGGGAGCT | |
| | CAAAATGGAGGACGCGGCGCTCGGGAGAGCGGGCGGGTGAGTCACC | |
| | CACACAAAGGAAAAGGGCCTTTCCGTCCTCAGCCGTCGCTTCATGT | |
| | GACTCCACGGAGTACCGGGCGCCGTCCAGGCACCTCGATTAGTTCT | |
| | CGAGCTTTTGGAGTACGTCGTCTTTAGGTTGGGGGGAGGGGTTTTA | |
| | TGCGATGGAGTTTCCCCACACTGAGTGGGTGGAGACTGAAGTTAGG | |
| | CCAGCTTGGCACTTGATGTAATTCTCCTTGGAATTTGCCCTTTTTG | |
| | AGTTTGGATCTTGGTTCATTCTCAAGCCTCAGACAGTGGTTCAAAG | |
| | TTTTTTTCTTCCATTTCAGGTGTCG | |
| copGFP | AGAGCGACGAGAGCGGCCTGCCCGCCATGGAGATCGAGTGCCGCAT | 50 |
| | CACCGGCACCCTGAACGGCGTGGAGTTCGAGCTGGTGGGCGGCGGA | |
| | GAGGGCACCCCCAAGCAGGGCCGCATGACCAACAAGATGAAGAGCA | |
| | CCAAAGGCGCCCTGACCTTCAGCCCCTACCTGCTGAGCCACGTGAT | |
| | GGGCTACGGCTTCTACCACTTCGGCACCTACCCCAGCGGCTACGAG | |
| | AACCCCTTCCTGCACGCCATCAACAACGGCGGCTACACCAACACCC | |
| | GCATCGAGAAGTACGAGGACGGCGGCGTGCTGCACGTGAGCTTCAG | |
| | CTACCGCTACGAGGCCGGCCGCGTGATCGGCGACTTCAAGGTGGTG | |
| | GGCACCGGCTTCCCCGAGGACAGCGTGATCTTCACCGACAAGATCA | |
| | TCCGCAGCAACGCCACCGTGGAGCACCTGCACCCCATGGGCGATAA | |
| | CGTGCTGGTGGGCAGCTTCGCCCGCACCTTCAGCCTGCGCGACGGC | |
| | GGCTACTACAGCTTCGTGGTGGACAGCCACATGCACTTCAAGAGCG | |
| | CCATCCACCCCAGCATCCTGCAGAACGGGGGCCCCATGTTCGCCTT | |
| | CCGCCGCGTGGAGGAGCTGCACAGCAACACCGAGCTGGGCATCGTG | |
| | GAGTACCAGCACGCCTTCAAGACCCCCATCGCCTTCGCCAGATCCC | |
| | GCGCTCAGTCGTCCAATTCTGCCGTGGACGGCACCGCCGGACCCGG | |
| | CTCCACCGGATCTCGC | |
| T2A | GAGGGCAGAGGAAGTCTTCTAACATGCGGTGACGTGGAGGAGAATC | 51 |
| | CCGGCCCT | |
| Puromycin | ATGACCGAGTACAAGCCCACGGTGCGCCTCGCCACCCGCGACGACG | 52 |
| Resistance | TCCCCAGGGCCGTACGCACCCTCGCCGCCGCGTTCGCCGACTACCC | |
| | CGCCACGCGCCACACCGTCGATCCGGACCGCCACATCGAGCGGGTC | |
| | ACCGAGCTGCAAGAACTCTTCCTCACGCGCGTCGGGCTCGACATCG | |
| | GCAAGGTGTGGGTCGCGGACGACGGCGCCGCGGTGGCGGTCTGGAC | |
| | CACGCCGGAGGGCGTCGAAGCGGGGGCGGTGTTCGCCGAGATCGGC | |
| | CCGCGCATGGCCGAGTTGAGCGGTTCCCGGCTGGCCGCGCAGCAAC | |
| | AGATGGAAGGCCTCCTGGCGCCGCACCGGCCCAAGGAGCCCGCGTG | |
| | GTTCCTGGCCACCGTCGGCGTCTCGCCCGACCACCAGGGCAAGGGT | |
| | CTGGGCAGCGCCGTCGTGCTCCCCGGAGTGGAGGCGGCCGAGCGCG | |
| | CCGGGGTGCCCGCCTTCCTGGAGACCTCCGCGCCCCGCAACCTCCC | |
| | CTTCTACGAGCGGCTCGGCTTCACCGTCACCGCCGACGTCGAGGTG | |
| | CCCGAAGGACCGCGCACCTGGTGCATGACCCGCAAGCCCGGTGCCT | |
| | GA | |
| MYRF | AGAGGCTCTCGCCCAGCCAGCCACAGACTACCACTTCCACTTCTAC | 53 |
| Left | CGCCTGTGTGACTGAGCTGCCCTCCTGAGGCAGCACCACACCAGGG | |
| Homology | ACCAGGGGTGCCCAGGCACCCCCCAACACTGGATGCAATGGTGTTA | |
| Arm: | CACTGGAGCCCGCTGCAGGCCAGCTCTGCTGTTCACTGGCCCTACC | |
| | CGAGACTGGTGAAACTGGAAGTCTTCACACTGGAGTTGCTGTTCCA | |
| | GCTGGTCGCCCCTCACGGCACAGAGGGAACCTGAGAGCCAGAGACT | |
| | TCTTGGGCCTTCCTGCCTGCCACCCCCTAGGGGCCAGGACAGGACC | |
| | AGTTTACCTCTTTCCAGATATGGTGGTTGGAGGGCTGGTTCAGGTG | |
| | CCCTGGAGGGAAGGGGAAGCCTGTGGCCCTGATTTGTTCAGAGCCC | |
| | ATTCTCCCTTGCCTCCCCTTTTGAGACTGGAGCCAACCCTTTTGGA | |
| | GAGAGGACCTGCCCACCTTTGAGATCAGCAGGGGGCTCGGATCCAG | |
| | CCCTAAGAGACTTGGGTGGACCCCCATGAGTCAATGGAGGGCAGAC | |
| | GGCTCTCCCCCTTAAAGCTGTTCCCTGGGGGATGGCTTGGTAGTGG | |
| | ACTTTCTGGGGTTTGCCTGTTACGCCAGACTCGGACTTCTAAGCTT | |
| | TAAGTGTGGCCCAGGAGGTTTCTTCTCCCTGGGAGGGCTTGGCTCC | |
| | CAAGAAGTCCCA | |

### Example 3—Human U251 Glioma Cells Expressing PD-L1 and CD47 Expand and Persist Preferentially in Immune-Humanized Hosts

### Materials and Methods:

Construction of the Targeting Plasmid: The targeting vector was generated using basic molecular coning techniques with PCR-generated inserts. Coding sequences for human PD-L1 (NCBI Reference Sequence: NM_014143.4, which is hereby incorporated by reference in its entirety), human CD47 (NCBI Reference Sequence: NM_001777.3, which is hereby incorporated y reference in its entirety), or EGFP were cloned immediately downstream of the internal ribosome entry site (IRES) in pIRES-hPGK-Puro-WPRE-BGHpa. Two shRNAs, targeting CIITA and B2M were also cloned immediately after PDL1 or CD47 (Table 15).

TABLE 15
shRNA Sequences

| | | SEQ |
|---|---|---|
| Name | Nucleotide Sequence | ID NO. |
| CIITA | 5′-CCG GAG GGC CTG AGC AAG GAC ATT TCT CGA GAA | 54 |
| | ATG TCC TTG CTC AGG CCC TTT TTT G-3′ | |
| | (TRCN0000299016, Sigma) | |
| B2M | 5′-CCG GCT GGT CTT TCT ATC TCT TGT ACT CGA GTA CAA | 55 |
| | GAG ATA GAA AGA CCA GTT TTT G-3′ | |
| | (TRCN0000230865, Sigma) | |

The homology arm overlapping the last coding exon was cloned from HEK293 cell genomic DNA. The left homology arm consisted of 842 bp (NCBI Reference Sequence: NC_000004.12 spanning from 54294436-54295277, which is hereby incorporated by reference in its entirety), while the right homology arm consisted of 875 bp (NCBI Reference Sequence: NC_000004.12 spanning from 54295286-54296160, which is hereby incorporated by reference in its entirety).

sgRNA (5′-CTG TAA CTG GCG GAT TCG AGG-3′; SEQ ID NO: 56) was cloned downstream of U6 promoter in pU6-PDGFRA2-CBh-Cas9-T2A-mCherry (Addgene plasmid #64324) and validated using the Surveyor nuclease assay in HEK293 cells (Surveyor Mutation Detection Kit, IDT).

Cell Transfection and Selection. U251 human malignant glioblastoma cells were maintained at 37° C., in 5% CO₂ in Dulbecco's modified Eagle's medium (DMEM; Invitrogen, Carlsbad, Calif., USA), supplemented with 10% heat-inactivated fetal bovine serum (FBS) and 1% penicillin-streptomycin (100 units/mL penicillin, and 100 μg/mL streptomycin).

U251 cells (5×10⁵) were transfected with 2 μg DNA mixture of targeting and sgRNA/Cas9 plasmids (1:1 ratio) using 4D Nucleofector™ (Lonza) with the SE Cell Line 4D-Nucleofector™ X transfection kit, following the DS-126 protocol and instructions supplied by the manufacturer. Three days post-transfection, the cells were passaged and cultured in puromycin-containing (1.5 μg/ml; Sigma) media for selection. Individual clones were expanded and genotyped for correct integration, integrity of transgene and absence of donor bacterial plasmids.

Selected clones were transduced with lentivirus expressing luciferase (pTANK-CMV-Luciferase-IRES-mCherry-WPRE; MOI=5). For transplantation, the cells were collected by trypsinization and concentrated to 1×10⁷ cells/ml in Hanks' Balanced Salt solution.

Animals, Cell Transplant and Imaging. Female huPBMC-NOG mice (NOD.Cg-Prkdc^{scid} Il2rg^{tm1Sug}/JicTac) were purchased from Taconic. The mice were housed (3-4 mice per cage) in germ-free environment. Transplantation was performed under 2.5% isoflurane anesthesia. A total of 1×10⁶ cells in 100 μl of HBSS was injected subcutaneously and unilaterally into the flank of mice.

Bioluminescence Imaging In Vivo. Bioluminescence imaging was performed on the IVIS® Spectrum imaging station (PerkinElmer) under 2.5% isoflurane anesthesia. At the time of Imaging of mice were given an injection of D-luciferin (150 mg/kg of body weight, i.p.; Sigma) 10 minute before imaging. Luminescence was calculated using IVIS® Spectrum software.

### Results:

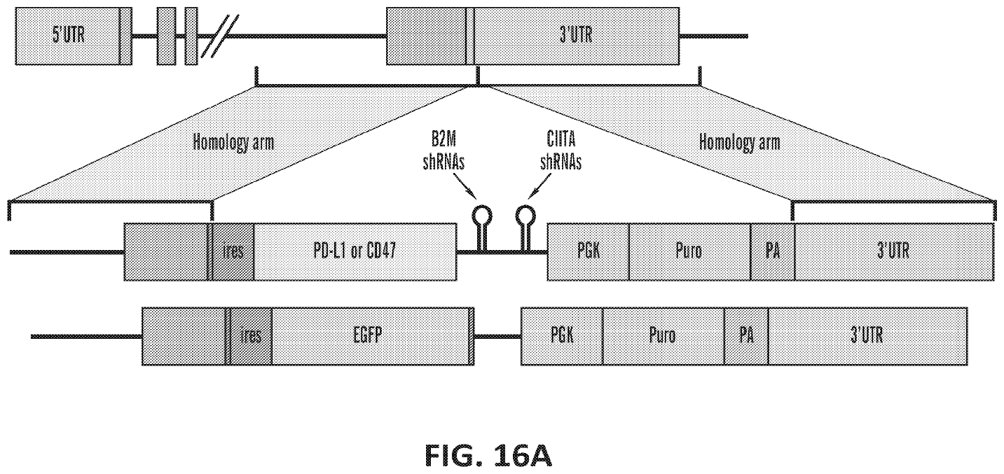

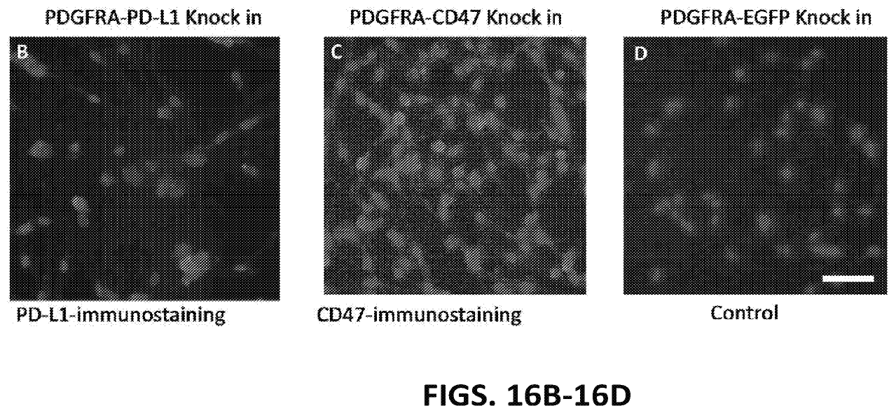

Generation of Recombinant Genetic Knock-In Constructs Expressing PD-L1, CD47, and EGFP cDNA with Target Sequences for the PDGFRA Locus. A schematic illustration of recombinant genetic constructs comprising a PD-L1 or CD47 knock-in vector targeting the PDGFRA gene locus is shown in FIG. 16A. The PD-L2 and CD47 knock-in vectors comprises, 5′→3′, a 5′ homology arm, a stop codon, an internal ribosomal entry site (IRES), a nucleotide sequence encoding CD47 or PD-L1, a nucleotide sequence encoding anti-B2M shRNA, a nucleotide sequence encoding anti-CIITA shRNA, a puromycin selection marker, and a 3′ homology arm. The EGFP vector (control vector) comprises, 5′-3′, a 5′ homology arm, a stop codon, an IRES, a nucleotide sequence encoding enhanced Green Fluorescent Protein (EGFP), a stop codon, a puromycin selection marker, and a 3′ homology arm. The puromycin selection markers in these constructs comprise a phosphoglycerate kinase (PGK) promoter and a polyadenylation signal (PA) for constitutive expression in mammalian cells. The CD47 and PD-L1 knock-in vectors enable knockdown of the Class I and II major histocompatibility complexes via shRNAi suppression of beta2-microglobulin and CIITA, the class 2 transactivator (FIG. 16A, top construct). The EGFP knock in vector (control vector) expresses only EGFP in pace of CD47 or PDL1, and does not express either shRNA (FIG. 16A, bottom construct). FIGS. 16B-16D show validation by immunostaining of clones generated via CRISPR-mediated knock-in of the recombinant genetic constructs of FIG. 16A into the PDGFRA locus, after puromycin selection and clonal expansion.

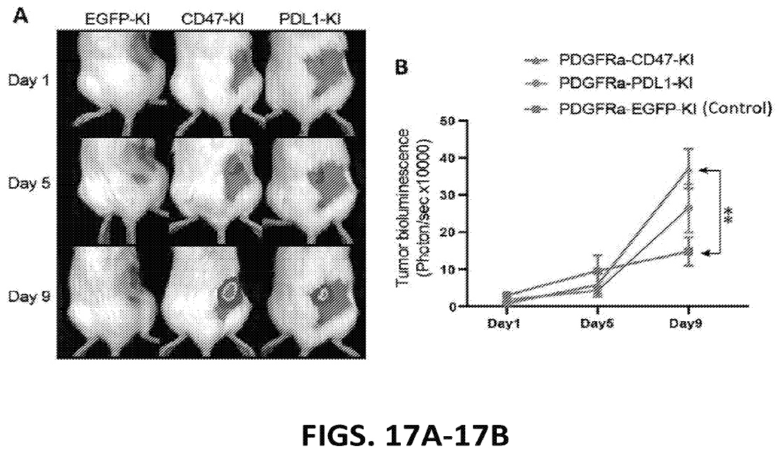

Human U251 Glioma Cells Expressing PD-L1 and CD47 Expand and Persist Preferentially in Immune-Humanized Hosts. Like their related glial progenitor cells, U251 cells express PDGFRA. On that basis, genetically-edited U251 knock-in (KI) cells expressing PD-L1 or CD47 or EGFP (control) in the PDGFRA gene locus were injected subcutaneously into the flank of huPBMC-NOG mice (human Peripheral Blood Mononuclear Cell-chimerized immunodeficient NOG mice). Tumor growth was monitored by in vivo bioluminescent imaging at 1-, 5-, or 9-days post-graft (FIG. 17A). By 9 days post graft, CD47-expressing U251 cells had expanded and persisted to a significantly greater extent than did EGFP-expressing control cells (FIG. 17B), consistent with their avoidance of graft rejection by the humanized host immune system.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A recombinant genetic construct comprising:
a first gene sequence expressed in a cell-type specific manner;
one or more immune checkpoint protein encoding nucleotide sequences positioned 3′ to the first gene sequence, and
a second gene sequence expressed in a cell-type specific manner, said second gene sequence located 3′ to the immune checkpoint protein encoding nucleotide sequences.

2. The recombinant genetic construct of claim 1 further comprising:
a nucleotide sequence encoding one or more agents that reduce expression of one or more HLA-I molecules, wherein said nucleotide sequence is coupled to the one or more immune checkpoint protein encoding nucleotide sequences.

3. A recombinant genetic construct comprising:
a first gene sequence expressed in a cell-type specific manner;
a nucleotide sequence encoding one or more agents that reduce expression of one or more HLA-I molecules, said nucleotide sequence positioned 3′ to the first cell specific gene sequence; and
a second gene sequence expressed in a cell-type specific manner, said second gene sequence positioned 3′ to the nucleotide sequence encoding one or more agents that reduce expression of one or more HLA-I molecules.

4. The recombinant genetic construct of claim 1 or claim 2, wherein the one or more immune checkpoint proteins is selected from programmed death ligand 1 (PD-L1), programmed death ligand 2 (PD-L2), CD47, CD200, CTLA-4, HLA-E, and any combination thereof.

5. The recombinant genetic construct of any one of claims 2-4, wherein the one or more agents that reduce expression of the one or more HLA-I molecules is selected from the group consisting of shRNA, miRNA, and siRNA.

6. The recombinant genetic construct of any one of claims 2-4, wherein the one or more agents that reduce expression of the one or more HLA-I molecules is a nuclease-deficient Cas9 or zinc-finger nuclease.

7. The recombinant genetic construct of any one of claims 2-6, wherein the one or more agents that reduce expression of the one or more HLA-I molecules is an agent that reduces expression of β₂M.

8. The recombinant genetic construct of any one of claims 2-6, wherein the one or more HLA-I molecules is selected from the group consisting of HLA-A, HLA-B, HLA-C, HLA-E, HLA-F, HLA-G, and combinations thereof.

9. The recombinant genetic construct of any one of claims 1-8, wherein the first and second gene sequences of the recombinant genetic construct are from a gene that is restrictively expressed in one or more terminally differentiated cells.

10. The recombinant genetic construct of claim 9, wherein the terminally differentiated cell is an oligodendrocyte.

11. The recombinant genetic construct of claim 10, wherein the first and second gene sequences are from a gene selected from the group consisting of SOX10, MYRF, MAG, and MBP.

12. The recombinant genetic construct of claim 9, wherein the terminally differentiated cell is an astrocyte.

13. The recombinant genetic construct of claim 12, wherein the first and second gene sequences are from a gene selected from GFAP and AQP4.

14. The recombinant genetic construct of claim 9, wherein the terminally differentiated cell is a neuron.

15. The recombinant genetic construct of claim 14, wherein the first and second gene sequences are from a gene selected from the group consisting of SYN1, MAP2, and ELAV4.

16. The recombinant genetic construct of claim 14, wherein the terminally differentiated cell is a dopaminergic neuron and the first and second gene sequences are from a gene selected from TH and DDC.

17. The recombinant genetic construct of claim 14, wherein the terminally differentiated cells are medium spiny neurons and cortical interneurons and the first and second gene sequences are from a gene selected from GAD65 and GAD67.

18. The recombinant genetic construct of claim 14, wherein the terminally differentiated cell is a cholinergic neuron and the first and second gene sequences are from CHAT.

17. The recombinant genetic construct of any one of claims 1-16 further comprising:
a further nucleotide sequence encoding one or more agents that reduce expression of one or more HLA-II molecules, wherein said further nucleotide sequence of the construct is coupled to the one or more immune checkpoint protein encoding nucleotide sequences and/or the nucleotide sequence encoding one or more agents that reduce expression of one or more HLA-I molecules.

18. The recombinant genetic construct of claim 17, wherein the one or more agents that reduce expression of one or more HLA-II molecules is selected from the group consisting of shRNA, miRNA, and siRNA.

19. The recombinant genetic construct of claim 17, wherein the one or more agents that reduce expression of one or more HLA-II molecules is a nuclease deficient Cas9 protein or zinc-finger nuclease.

20. The recombinant genetic construct of claim 17, wherein the one or more agents that reduce expression of the one or more HLA-II molecules is an agent that reduces expression of class II major histocompatibility complex transactivator (CIITA).

21. The recombinant genetic construct of any one of claims 1-20 further comprising:
one or more self-cleaving peptide encoding nucleotide sequences, wherein said self-cleaving peptide encoding nucleotide sequences are positioned within the construct in a manner effective to mediate translation of the one or more immune checkpoint proteins.

22. The recombinant genetic construct of claim 21, wherein the self-cleaving peptide is selected from the group consisting of porcine teschovirus-1 2A (P2A), those assign a virus 2A (T2A), equine rhinitis A virus 2A (E2A), cytoplasmic polyhedrosis virus (BmCPV 2A), and flacherie virus (BmIFV 2A).

23. The recombinant genetic construct of any one of claims 1-22 further comprising:
an inducible cell death gene positioned within the construct in a manner effective to achieve inducible cell suicide.

24. The recombinant genetic construct of claim 22, wherein the inducible cell death gene is selected from caspase-3, caspase-9, and thymidine kinase.

25. A preparation of one or more cells, wherein cells of the preparation comprise the recombinant genetic construct of any one of claims 1-24.

26. The preparation of claim 25, wherein cells of the preparation are mammalian cells.

27. The preparation of claim 25, wherein cells of the preparation are human cells.

28. The preparation of claim 25, wherein cells of the preparation are pluripotent cells.

29. The preparation of claim 28, wherein the pluripotent cells are induced pluripotent stem cells.

30. The preparation of claim 28, wherein the pluripotent cells are embryonic stem cells.

31. The preparation of claim 25, wherein cells of the preparation are progenitor cells.

32. The preparation of claim 31, wherein the progenitor cells are glial progenitor cells.

33. The preparation of claim 31, wherein the progenitor cells are oligodendrocyte-biased progenitor cells.

34. The preparation of claim 31, wherein the progenitor cells are astrocyte-biased progenitor cells.

35. The preparation of claim 31, wherein the progenitor cells are neuronal progenitor cells.

36. The preparation of claim 25, wherein cells of the preparation are terminally differentiated cells.

37. The preparation of claim 36, wherein the terminally differentiated cells are neurons, oligodendrocytes, or astrocytes.

38. A method comprising:
administering the preparation of any one of claims 25-37 to a subject in need thereof.

39. A method of treating a subject having a condition mediated by a loss of myelin or by dysfunction or loss of oligodendrocytes, said method comprising:
administering to the subject a preparation of claim 32 or claim 33 under conditions effective to treat the condition.

40. A method of treating a subject having a condition mediated by dysfunction or loss of astrocytes, said method comprising:
administering to the subject a preparation of claim 32 or claim 34 under conditions effective to treat the condition.

41. A method of treating a subject having a condition mediated by dysfunction or loss of neurons, said method comprising:
administering to the subject a preparation of claim 31 or claim 35 under conditions effective to treat the condition.

42. The method of any one of claims 39-41, wherein the preparation is administered to one or more sites of the brain, the brain stem, the spinal cord, or a combination thereof.

43. The method of claim 42, wherein the preparation is administered intraventricularly, intracallosally, or intraparenchymally.

44. A preparation of one or more cells, wherein cells of the preparation are modified to conditionally express:
(i) increased levels of one or more immune checkpoint proteins as compared to corresponding wild-type cells,
(ii) reduced levels of one or more HLA-I proteins as compared to corresponding wild-type cells, or
(iii) a combination of (i) and (ii).

45. The preparation of claim 44, wherein the modified cells of the preparation are terminally differentiated cells.

46. The preparation of claim 44, wherein the one or more HLA-I proteins are selected from the group consisting of HLA-A, HLA-B, HLA-C, HLA-E, HLA-F, HLA-G, and combinations thereof.

47. The preparation of claim 44, wherein the one or more immune checkpoint proteins are selected from programmed death ligand 1 (PD-L1), programmed death ligand 2 (PD-L2), CD47, CD200, CTL4A, HLE-1, and any combination thereof.

48. The preparation of any one of claims 44-47, wherein modified cells of the preparation conditionally express reduced levels of one or more HLA-II proteins as compared to corresponding wild-type cells.

49. The preparation of cells according to claim 48, wherein the one or more HLA-II proteins are selected from the group consisting of HLA-DM, HLA-DO, HLA-DP, HLA-DQ, HLA-DR, and combinations thereof.

50. A method of generating a conditionally immunoprotected cell, said method comprising:
modifying a cell to conditionally express (i) increased levels of one or more immune checkpoint proteins; (ii) one or more agents that reduce expression of one or more HLA-I proteins; or (iii) both (i) and (ii).

51. The method of claim 50, wherein the conditional expression of the one or more immune checkpoint proteins and the conditional expression of the one or more agents that reduce expression of one or more HLA-I molecules are operably coupled to a gene that is restrictively expressed in a terminally differentiated cell.

52. The method of claim 51, wherein the terminally differentiated cell is an oligodendrocyte.

53. The method of claim 52, wherein the gene that is restrictively expressed in the oligodendrocyte is selected from the group consisting of SOX10, MYRF, MAG, and MBP.

54. The method of claim 51, wherein the terminally differentiated cell is an astrocyte.

55. The method of claim 54, wherein gene that is restrictively expressed in the astrocyte is GFAP or AQP4.

56. The method of claim 51, wherein the terminally differentiated cell is a neuron.

57. The method of claim 56, wherein the gene that is restrictively expressed in the neuron is selected from the group consisting of SYN1, MAP2, and ELAV4.

58. The recombinant genetic construct of claim 51, wherein the terminally differentiated cell is a dopaminergic neuron and the gene that is restrictively expressed in the dopaminergic neuron is TH or DDC.

59. The recombinant genetic construct of claim 51, wherein the terminally differentiated cells are medium spiny neurons and cortical interneurons and the gene that is restrictively expressed in the medium spiny neurons and cortical interneurons is GAD65 or GAD67.

60. The recombinant genetic construct of claim 51, wherein the terminally differentiated cell is a cholinergic neuron and the gene that is restrictively expressed in the cholinergic neuron is acetylcholine transferase.

61. The method of claim 50, wherein the one or more immune checkpoint proteins are selected from programmed death ligand 1 (PD-L1), programmed death ligand 2 (PD-L2), CD47, CD200, CTLA4, HLE-A, and any combination thereof

62. The method of claim 50, wherein the one or more HLA-I proteins are selected from the group consisting of HLA-A, HLA-B, HLA-C, HLA-E, HLA-F, HLA-G, and combinations thereof.

63. The method of claim 50, wherein the one or more agents that reduce expression of one or more HLA-I proteins is selected from the group consisting of shRNA, miRNA, and siRNA.

64. The method of claim 50, wherein the one or more agents that reduce expression of one or more HLA-I proteins is nuclease-deficient CRISPR-Cas9 protein or a zinc-finger nuclease.

65. The method of claim 50, wherein the one or more agents that reduce expression of the one or more HLA-I molecules is an agent that reduces expression of β₂M.

66. The method of claim 50 further comprising:
modifying the cell to conditionally express one or more agents that reduce expression of one or more HLA-II molecules.

67. The method according to claim 66, wherein the one on more agents that reduce expression of the one or more HLA-II molecules is an agent that reduces expression of class II major histocompatibility complex transactivator (CIITA).

68. The method of claim 62, wherein the one or more agents that reduce expression of one or more HLA-II molecules is selected from the group consisting of shRNA, miRNA, and siRNA.

69. The method of claim 62, wherein the one or more agents that reduce expression of one or more HLA-II proteins is nuclease-deficient CRISPR-Cas9 protein or a zinc-finger nuclease.

70. The method of any one of claims 50-69, wherein the conditionally immunoprotected cell is a mammalian cell.

71. The method of 70, wherein the conditionally immunoprotected mammalian cell is a human cell.

72. The method of any one of claims 50-69, wherein the conditionally immunoprotected cell is a pluripotent cell.

73. The method of claim 72, wherein the conditionally immunoprotected pluripotent cell is an induced pluripotent stem cell.

74. The method of claim 73, wherein the conditionally immunoprotected pluripotent cell is an embryonic stem cell.

75. The method of any one of claims 50-69, wherein the conditionally immunoprotected cell is a progenitor cell.

76. The method of claim 75, wherein the conditionally immunoprotected progenitor cell is a glial progenitor cell.

77. The method of claim 75, wherein the conditionally immunoprotected progenitor cell is an oligodendrocyte-biased progenitor cell.

78. The method of claim 75, wherein the conditionally immunoprotected progenitor cell is an astrocyte-biased progenitor cell.

79. The method of claim 50, wherein the modifying comprises:
(i) introducing into the cell a sequence-specific nuclease that cleaves a target gene at a position upstream of its 3′ untranslated region (UTR), wherein said target gene is a gene expressed in a cell-specific manner and
(ii) introducing into the cell a recombinant genetic construct comprising:
(a) one or more immune checkpoint proteins encoding nucleotide sequences;
(b) a nucleotide sequence encoding one or more agents that reduce expression of one or more HLA-I molecules; or
(c) both (a) and (b) wherein the recombinant genetic construct is inserted into the target gene at the nuclease cleavage site through homologous recombination.

80. The method of claim 79, wherein the sequence-specific nuclease is selected from the group consisting of zinc finger nuclease (ZFN), transcription activator-like effector nuclease (TALEN), and an RNA-guided nuclease.

81. The method of claim 80, wherein the sequence-specific nuclease is a RNA-guided nuclease in the form of Cas9.

82. The method according to claim 79, wherein the sequence-specific nuclease is introduced into the cell as a protein, mRNA, or cDNA.


SEQUENCE LISTING (56 sequences)

SEQ ID NO: 1 - DNA, 531 bp, Homo sapiens
atgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 2 - DNA, 873 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 3 - DNA, 822 bp, Homo sapiens
atgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttggctgcttcacattttcatccccttctgcatcattgctttcattttcatagccacagtgatagccctaagaaaacaactctgtcaaaagctgtattcttcaaaagacacaacaaaaagacctgtcaccacaacaaagagggaagtgaacagtgctatctga

SEQ ID NO: 4 - DNA, 852 bp, Homo sapiens
atgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttggctgcttcacattttcatccccttctgcatcattgctttcattttcatagccacagtgatagccctaagaaaacaactctgtcaaaagctgtattcttcaaaagacacaacaaaaagacctgtcaccacaacaaagagggaagtgaacagtgctgtgaatctgaacctgtggtcttgggagccagggtga

SEQ ID NO: 5 - DNA, 972 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 6 - DNA, 918 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 7 - DNA, 882 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggaataa

SEQ ID NO: 8 - DNA, 916 bp, Artificial
  Homo sapiens clone ccsbBroadEn_13826 CD47 gene, encodes complete protein
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctggaataactg

SEQ ID NO: 9 - DNA, 810 bp, Homo sapiens
atggagaggctggtgatcaggatgcccttctctcatctgtctacctacagcctggtttgggtcatggcagcagtggtgctgtgcacagcacaagtgcaagtggtgacccaggatgaaagagagcagctgtacacacctgcttccttaaaatgctctctgcaaaatgcccaggaagccctcattgtgacatggcagaaaaagaaagctgtaagcccagaaaacatggtcaccttcagcgagaaccatggggtggtgatccagcctgcctataaggacaagataaacattacccagctgggactccaaaactcaaccatcaccttctggaatatcaccctggaggatgaagggtgttacatgtgtctcttcaatacctttggttttgggaagatctcaggaacggcctgcctcaccgtctatgtacagcccatagtatcccttcactacaaattctctgaagaccacctaaatatcacttgctctgccactgcccgcccagcccccatggtcttctggaaggtccctcggtcagggattgaaaatagtacagtgactctgtctcacccaaatgggaccacgtctgttaccagcatcctccatatcaaagaccctaagaatcaggtggggaaggaggtgatctgccaggtgctgcacctggggactgtgaccgactttaagcaaaccgtcaacaaaggctattggttttcagttccgctattgctaagcattgtttccctggtaattcttctcgtcctaatctcaatcttactgtactggaaacgtcaccggaatcaggaccgagagccctaa

SEQ ID NO: 10 - DNA, 885 bp, Homo sapiens
atggagaggctgactctgaccaggacaattgggggccctctccttacagctacactcctaggaaagaccaccatcaatgattaccaggtgatcaggatgcccttctctcatctgtctacctacagcctggtttgggtcatggcagcagtggtgctgtgcacagcacaagtgcaagtggtgacccaggatgaaagagagcagctgtacacacctgcttccttaaaatgctctctgcaaaatgcccaggaagccctcattgtgacatggcagaaaaagaaagctgtaagcccagaaaacatggtcaccttcagcgagaaccatggggtggtgatccagcctgcctataaggacaagataaacattacccagctgggactccaaaactcaaccatcaccttctggaatatcaccctggaggatgaagggtgttacatgtgtctcttcaatacctttggttttgggaagatctcaggaacggcctgcctcaccgtctatgtacagcccatagtatcccttcactacaaattctctgaagaccacctaaatatcacttgctctgccactgcccgcccagcccccatggtcttctggaaggtccctcggtcagggattgaaaatagtacagtgactctgtctcacccaaatgggaccacgtctgttaccagcatcctccatatcaaagaccctaagaatcaggtggggaaggaggtgatctgccaggtgctgcacctggggactgtgaccgactttaagcaaaccgtcaacaaaggctattggttttcagttccgctattgctaagcattgtttccctggtaattcttctcgtcctaatctcaatcttactgtactggaaacgtcaccggaatcaggaccgagagccctaa

SEQ ID NO: 11 - DNA, 462 bp, Homo sapiens
atgaagggtgttacatgtgtctcttcaatacctttggttttgggaagatctcaggaacggcctgcctcaccgtctatgcccatagtatcccttcactacaaattctctgaagaccacctaaatatcacttgctctgccactgcccgcccagcccccatggtcttctggaaggtccctcggtcagggattgaaaatagtacagtgactctgtctcacccaaatgggaccacgtctgttaccagcatcctccatatcaaagaccctaagaatcaggtggggaaggaggtgatctgccaggtgctgcacctggggactgtgaccgactttaagcaaaccgtcaacaaaggctattggttttcagttccgctattgctaagcattgtttccctggtaattcttctcgtcctaatctcaatcttactgtactggaaacgtcaccggaatcaggaccgagagccctaa

SEQ ID NO: 12 - DNA, 588 bp, Homo sapiens
atggtcaccttcagcgagaaccatggggtggtgatccagcctgcctataaggacaagataaacattacccagctgggactccaaaactcaaccatcaccttctggaatatcaccctggaggatgaagggtgttacatgtgtctcttcaatacctttggttttgggaagatctcaggaacggcctgcctcaccgtctatgtacagcccatagtatcccttcactacaaattctctgaagaccacctaaatatcacttgctctgccactgcccgcccagcccccatggtcttctggaaggtccctcggtcagggattgaaaatagtacagtgactctgtctcacccaaatgggaccacgtctgttaccagcatcctccatatcaaagaccctaagaatcaggtggggaaggaggtgatctgccaggtgctgcacctggggactgtgaccgactttaagcaaaccgtcaacaaaggctattggttttcagttccgctattgctaagcattgtttccctggtaattcttctcgtcctaatctcaatcttactgtactggaaacgtcaccggaatcaggaccgagagccctaa

SEQ ID NO: 13 - DNA, 672 bp, Homo sapiens
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 14 - DNA, 672 bp, Homo sapiens; identical to SEQ ID NO: 13

SEQ ID NO: 15 - DNA, 1077 bp, Homo sapiens
atggtagatggaaccctccttttactctcctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 16 - DNA, 1077 bp, Homo sapiens
atggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcttctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 17 - DNA, 1077 bp, Homo sapiens
atggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 18 - DNA, 360 bp, Homo sapiens
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 19 - DNA, 360 bp, Homo sapiens
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatttaa

SEQ ID NO: 20 - DNA, 360 bp, Homo sapiens; identical to SEQ ID NO: 18

SEQ ID NO: 21 - DNA, 360 bp, Homo sapiens; identical to SEQ ID NO: 18

SEQ ID NO: 22 - DNA, 4543 bp, Homo sapiens
tgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacgccttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgcttgagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcggcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatgatcccagctgtgctctggacaggcatgttctctgaggacactaaccacgctggaccttgaactgggtacttgtggacacagctcttctccaggctgtatcccatgaggcctcagcatcctggcacccggcccctgctggttcagggttggcccctgcccggctgcggaatgaaccacatcttgctctgctgacagacacaggcccggctccaggctcctttagcgcccagttgggtggatgcctggtggcagctgcggtccacccaggagccccgaggccttctctgaaggacattgcggacagccacggccaggccagagggagtgacagaggcagccccattctgcctgcccaggcccctgccaccctggggagaaagtacttctttttttttatttttagacagagtctcactgttgcccaggctggcgtgcagtggtgcgatctgggttcactgcaacctccgcctcttgggttcaagcgattcttctgcttcagcctcccgagtagctgggactacaggcacccaccatcatgtctggctaatttttcatttttagtagagacagggttttgccatgttggccaggctggtctcaaactcttgacctcaggtgatccacccacctcagcctcccaaagtgctggggattacaagcgtgagccactgcaccgggccacagagaaagtacttctccaccctgctctccgaccagacaccttgacagggcacaccgggcactcagaagacactgatgggcaacccccagcctgctaattccccagattgcaacaggctgggcttcagtggcaggctgcttttgtctatgggactcaatgcactgacattgttggccaaagccaaagctaggcctggccagatgcaccaggcccttagcagggaaacagctaatgggacactaatggggcggtgagaggggaacagactggaagcacagcttcatttcctgtgtcttttttcactacattataaatgtctctttaatgtcacaaaaaaaaaaaaaaaaaaaaaaa

SEQ ID NO: 23 - DNA, 5356 bp, Homo sapiens
cctcccaactggtgactggttagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacgccttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgcttgagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcaggtggggtgaggggcttggaagagacatccttgtgttgggcattaactgcggtcttggtgccaagcccagtgctctgtggggtccttttagtatgcagagcagccgggtggggcagaatggattctctccatttttaagatgaggatgttgaggctcagagaggggcagccacttgccacacagcaagtgagaggcaatggcattctcccagtcaatatttgaaggcccgccatgtgccagtcactggggtatgtctagaatctgagactgacctgggctcaaatttgttttattctttccaccccctgagcacgccaccgttttcttatgctaagagtaaagccatggcctccccttggactctctgcctccattctctcctcttccactccattttgtattcagcaaccagaccaatcttctcagaacttgaatctgattgtatcccatccctgcttacaatccttcagggacactccaccactgtcaggatgaaggctaaatttcttaatttggtttcattaagtcggtctgcaatctgcttgagcatttcagcttaatcgccagaggattgcttccatatttccccctaaacatactttacccaagctgtaaggtcctacataattgtgccaataatttagcagtgagcttcctggtagccgaagcaaaaagggaaagaaaaccactgtgtgagttgtgagaaagtaggaatcaataaaggctggagtggtcgctgccttgagcgacacggtggcgatggaaggctttctgggaaaggtagaggttgagctaaggaaagaaagtattttaataggtaggaggacccttcatggagctgcccttccattaaggtctagcctggtcaccgtgcctgggtctgaggccctccctccacaggctgtgggagtccctgcggcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatgatcccagctgtgctctggacaggcatgttctctgaggacactaaccacgctggaccttgaactgggtacttgtggacacagctcttctccaggctgtatcccatgagcctcagcatcctggcacccggcccctgctggttcagggttggcccctgcccggctgcggaatgaaccacatcttgctctgctgacagacacaggcccggctccaggctcctttagcgcccagttgggtggatgcctggtggcagctgcggtccacccaggagccccgaggccttctctgaaggacattgcggacagccacggccaggccagagggagtgacagaggcagccccattctgcctgcccaggcccctgccaccctggggagaaagtacttctttttttttatttttagacagggtctcactgttgcccaggctggcgtgcagtggtgcgatctgggttcactgcaacctccgcctcttgggttcaagcgattcttctgcttcagcctcccgagtagctgggactacaggcacccaccatcatgtctggctaatttttcatttttggtagagacagggttttgccgtgttggccgggctggtctcgaactcttgacctcgggtgatccacccacctcagcctcccaaagtgctgggattacaagcgtgagccactgcaccgggccacagagaaagtacttctccaccctgctctccgaccagacaccttgacagggcacaccgggcactcagaagacactgatgggcaacccccagcctgctaattccccagattgcaacaggctgggcttcagtggcagctgcttttgtctatgggactcaatgcactgacattgttggccaaagccaaagctaggcctggccagatgcaccagcccttagcagggaaacagctaatgggacactaatggggcggtgagaggggaacagactggaa

SEQ ID NO: 24 - DNA, 66 bp, Artificial
  self-cleaving peptide
ggaagcggagctactaacttcagcctgctgaagcaggctggagacgtggaggagaaccctggacct

SEQ ID NO: 25 - DNA, 66 bp, Artificial
  self-cleaving peptide
ggttccggagccacgaacttctctctgttaaagcaagcaggagacgtggaagaaaaccccggtccc

SEQ ID NO: 26 - DNA, 75 bp, Artificial
  self-cleaving peptide
ggaagcggagtgaaacagactttgaattttgaccttctcaagttggcgggagacgtggagtccaaccctggacct

SEQ ID NO: 27 - DNA, 54 bp, Artificial
  self-cleaving peptide
gagggcagaggaagtcttctaacatgcggtgacgtggaggagaatcccggccct

SEQ ID NO: 28 - DNA, 69 bp, Artificial
  self-cleaving peptide
ggaagcggacagtgtactaattatgctctcttgaaattggctggagatgttgagagcaaccctggacct

SEQ ID NO: 29 - DNA, 66 bp, Cytoplasmic polyhedrosis virus
gacgtttttcgctctaattatgacctactaaagttgtgcggtgatatcgagtctaatcctggacct

SEQ ID NO: 30 - DNA, 66 bp, Artificial
  self-cleaving peptide
actctgacgagggcgaagattgaggatgaattgattcgtgcaggaattgaatcaaatcctggacct

SEQ ID NO: 31 - DNA, 600 bp, Artificial
  puromycin resistance selection marker
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagggcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 32 - DNA, 816 bp, Artificial
  neomycin resistance selection marker
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 33 - DNA, 1026 bp, Artificial
  Hygromycin B selection marker
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctcgagccggtcgcggaggcgatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcatgcggatttcggctccaacaatgtcctgacggacaatggccgcataacagcggtcattgactggagcgaggcgatgttcggggattcccaatacgaggtcgccaacatcctcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 34 - DNA, 1177 bp, Artificial
  UBC promoter sequence
ggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 35 - DNA, 511 bp, Artificial
  PGK promoter sequence
ttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacct

SEQ ID NO: 36 - DNA, 1179 bp, Artificial
  EF1a promoter sequence
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 37 - DNA, 589 bp, Artificial
  CMV promoter sequence
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 38 - DNA, 1718 bp, Artificial
  CAGG promoter sequence
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 39 - DNA, 344 bp, Artificial
  SV40 promoter sequence
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 40 - DNA, 752 bp, Artificial
  CopGFP
agagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgc

SEQ ID NO: 41 - DNA, 717 bp, Artificial
  eGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 42 - DNA, 720 bp, Artificial
  YFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 43 - DNA, 711 bp, Artificial
  mCherry
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 44 - DNA, 525 bp, Homo sapiens
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgctaaagaaaagaagccctcttacaacaggggtctatgtgaaaatgcccccaacagagccagaatgtga

SEQ ID NO: 45 - DNA, 23 bp, Artificial
  single guide RNA
tcaggccaactgcagttcagagg

SEQ ID NO: 46 - DNA, 713 bp, Artificial
  MYRF Right Homology Arm
ggtttgaatcccagctgtgtgattttgccacactgtgtgatttttaggaagtggctcagtttcctcatccagaagatggggctagtagcagcactgtgtcactggattgtactgaggatggggctaatgaaatactttgatgtgcccagagcatagtgggtgagggaacccagcacaacaggactgggaaggaggcaggggccaggtggaggtggctgtggacctgccagtcccgggcacggtctgcatggagtagctgccattgctccttctgccaaagcagaacatgctccttcctatctcttcaaagttctctgcttttttccttcataaaactccccacagaccccaggactgcgacggccgtggtgagagatgctggttgggataagggcagcagtctgtcctgacccctctctcccttctctccagggcacctctcaccggtggccaataaccatcctgtccttccgtgaattcacctaccacttccgggtggcactgctggtgagcaggggcatcccacctaccctggaggtctgggcacccctgtctgcgacgtggggcttgaggaatggggggtttgcacagtatgtggtagggctgggggcacagtgtcaagcaatgtcagcagggagtgccatctgccccgcacccccagagccacctcaccttcccactgcccttccacccagggtcaggccaactgcagt

SEQ ID NO: 47 - DNA, 66 bp, Artificial; identical to SEQ ID NO: 24
  P2A

SEQ ID NO: 48 - DNA, 972 bp, Artificial; identical to SEQ ID NO: 5
  Homo sapiens

SEQ ID NO: 49 - DNA, 1175 bp, Artificial
  EF1a promoter
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcg

SEQ ID NO: 50 - DNA, 752 bp, Artificial; identical to SEQ ID NO: 40
  CopGFP

SEQ ID NO: 51 - DNA, 54 bp, Artificial; identical to SEQ ID NO: 27
  T2A

SEQ ID NO: 52 - DNA, 600 bp, Artificial; identical to SEQ ID NO: 31
  Puromycin resistance marker

SEQ ID NO: 53 - DNA, 702 bp, Artificial
  MYRF Left Homology Arm
agaggctctcgcccagccagccacagactaccacttccacttctaccgcctgtgtgactgagctgccctcctgaggcagcaccacaccagggaccaggggtgcccaggcaccccccaacactggatgcaatggtgttacactggagcccgctgcaggccagctctgctgttcactggccctacccgagactggtgaaactggaagtcttcacactggagttgctgttccagctggtcgcccctcacggcacagagggaacctgagagccagagacttcttgggccttcctgcctgccaccccctaggggccaggacaggaccagtttacctctttccagatatggtggttggagggctggttcaggtgccctggagggaaggggaagcctgtggccctgatttgttcagagcccattctcccttgcctccccttttgagactggagccaacccttttggagagaggacctgcccacctttgagatcagcagggggctcggatccagccctaagagacttgggtggacccccatgagtcaatggagggcagacggctctcccccttaaagctgttccctgggggatggcttggtagtggactttctggggtttgcctgttacgccagactcggacttctaagctttaagtgtggcccaggaggtttcttctccctgggagggcttggctcccaagaagtccca

SEQ ID NO: 54 - DNA, 58 bp, Artificial
  shRNA targeting CIITA
ccggagggcctgagcaaggacatttctcgagaaatgtccttgctcaggcccttttttg

SEQ ID NO: 55 - DNA, 58 bp, Artificial
  shRNA targeting B2M
ccggctggtctttctatctcttgtactcgagtacaagagatagaaagaccagtttttg

SEQ ID NO: 56 - DNA, 21 bp, Artificial
  sgRNA
ctgtaactggcggattcgagg